(19) United States
(12) Patent Application Publication
(10) Pub. No.: US 2016/0310454 A1
(43) Pub. Date: Oct. 27, 2016

(54) LIVER X RECEPTOR AGONISTS IN THE TREATMENT OF EMPHYSEMA

(71) Applicants: Jeanine D'ARMIENTO, New York, NY (US); Piotr SKLEPKIEWICZ, Fort Lee, NJ (US); Vincent LEMAITRE, New York, NY (US)

(72) Inventors: Jeanine D'Armiento, New York, NY (US); Vincent Lemaitre, New York, NY (US)

(21) Appl. No.: 15/105,532

(22) PCT Filed: Dec. 17, 2014
(86) PCT No.: PCT/US2014/070941

§ 371 Date: Jun. 16, 2016

Related U.S. Application Data
(60) Provisional application No. 61/917,319, filed on Dec. 17, 2013.

(51) Int. Cl. A61K 31/18; G01N 33/92; A61K 31/195; C12N 15/113

(52) U.S. Cl. CPC A61K 31/18; C12N 15/113; G01N 33/92; A61K 31/195; G01N 2405/00; G01N 2405/08; G01N 2800/122; G01N 2800/52; C12N 2310/3233; C12N 2310/113; C12N 2320/30

(57) ABSTRACT

The present invention provides methods and compositions for treating a subject afflicted with chronic obstructive pulmonary disease (COPD) which comprise a i) a Liver X receptor (LXR) agonist, ii) a miR-33 antagonist, or iii) a TLR4/Myd88 pathway antagonist. The present invention also provides methods and compositions for use in prophylactically treating a subject for chronic obstructive pulmonary disease (COPD) which comprise i) a Liver X receptor (LXR) agonist, ii) a miR-33 antagonist, or iii) a TLR4/Myd88 pathway antagonist.

This application claims priority of U.S. Provisional Application No. 61/917,319, filed Dec. 17, 2013, the entire contents of which are hereby incorporated herein by reference.

This application incorporates-by-reference nucleotide and/or amino acid sequences which are present in the file named “141216_0575_85011-A-PCT_SequenceListing_REB.txt,” which is 0.80 kilobytes in size, and which was created Dec. 16, 2014 in the IBM-PC machine format, having an operating system compatibility with MS-Windows, which is contained in the text file filed December 16, 2014 as part of this application.

Throughout this application, various publications are referenced, including in parentheses. Full citations for publications referenced may be found listed at the end of the specification immediately preceding the claims. The disclosures of all referenced publications in their entireties are hereby incorporated by reference into this application in order to more fully describe the state of the art to which this invention pertains.

### BACKGROUND OF INVENTION

Chronic obstructive pulmonary disease (COPD) is the third leading cause of death in the United States (Podowski et al., 2012; Mannino et al., 2007) with tobacco smoke the key etiologic agent of this disease process; the inflammatory response to inhaled cigarette smoke and other noxious particles (Global Initiative for Chronic Obstructive Lung Disease, 2011; Global Initiative for Chronic Obstructive Lung Disease, 2007) is thought to be a primary initiator of the disease. COPD is characterized by progressive airflow limitation that is not fully reversible. A spectrum of pathological findings are observed in COPD ranging from inflammation of the larger airways (termed chronic bronchitis), remodeling of the small airways, and parenchymal tissue destruction with airspace enlargement (defined as emphysema) (Global Initiative for Chronic Obstructive Lung Disease, 2011; Global Initiative for Chronic Obstructive Lung Disease, 2007). In addition, COPD contributes to systemic manifestations affecting skeletal muscles, bone and the cardiovascular system (Yoshida et al., 2007, Celli et al., 2006). Despite the heterogeneity of COPD, the small airway walls in the emphysematous lung consistently demonstrate persistent inflammation with mononuclear phagocytes that play a major role in the inflammatory response (Shan et al., 2009; Shaykhiev et al., 2009).

New methods and compositions for treating COPD are needed.

### SUMMARY OF THE INVENTION

The present invention provides methods for treating a subject afflicted with chronic obstructive pulmonary disease (COPD) which comprises administering to the subject i) a Liver X receptor (LXR) agonist, ii) a miR-33 antagonist, or iii) a TLR4/Myd88 pathway antagonist in an amount that is effective to treat the subject.

The present invention provides methods for prophylactically treating a subject for chronic obstructive pulmonary disease (COPD) which comprises administering to the subject i) a Liver X receptor (LXR) agonist, ii) a miR-33 antagonist, or iii) a TLR4/Myd88 pathway antagonist in an amount that is effective to treat the subject.

The present invention provides methods for identifying whether a subject afflicted with chronic obstructive pulmonary disease (COPD) is responding to treatment for COPD comprising
i) periodically obtaining biological samples from the subject;
ii) assaying whether the level of a ceramide has increased or decreased in the biological samples over a period of time, and
iii) identifying the subject as responding to treatment if the level of the ceramide has decreased over the period of time.

The present invention provides methods for identifying whether a subject afflicted with chronic obstructive pulmonary disease (COPD) is responding to treatment for COPD comprising
i) periodically obtaining biological samples from the subject;
ii) assaying whether the level of sphingosine 1-phosphate (S1P) has increased or decreased in the biological samples over a period of time, and
iii) identifying the subject as responding to treatment if the level of S1P has increased over the period of time.

The present invention provides methods for determining whether chronic obstructive pulmonary disease (COPD) is progressing in a subject afflicted with COPD comprising
i) periodically obtaining biological samples from the subject;
ii) assaying whether the level of a ceramide has increased or decreased in the biological samples over a period of time, and
iii) identifying the COPD as progressing in the subject if the level of the ceramide has decreased over the period of time.

The present invention provides methods for determining whether chronic obstructive pulmonary disease (COPD) is progressing in a subject afflicted with COPD comprising
i) periodically obtaining biological samples from the subject;
ii) assaying whether the level of sphingosine 1-phosphate (S1P) has increased or decreased in the biological samples over a period of time, and
iii) identifying the COPD as progressing in the subject if the level of sphingosine 1-phosphate (S1P) has increased over the period of time.

The present invention provides compositions for use in treating a subject afflicted with chronic obstructive pulmonary disease (COPD) which comprises i) a Liver X receptor (LXR) agonist, ii) a miR-33 antagonist, or iii) a TLR4/Myd88 pathway antagonist.

The present invention provides compositions for use in prophylactically treating a subject for chronic obstructive pulmonary disease (COPD) which comprises i) a Liver X receptor (LXR) agonist, ii) a miR-33 antagonist, or iii) a TLR4/Myd88 pathway antagonist.

Aspects of the present invention relate to the use of i) a Liver X receptor (LXR) agonist, ii) a miR-33 antagonist, or iii) a TLR4/Myd88 pathway antagonist for the manufacture of a medicament for the treatment of chronic obstructive pulmonary disease (COPD).

Aspects of the present invention relate to the use of i) a Liver X receptor (LXR) agonist, ii) a miR-33 antagonist, or iii) a TLR4/Myd88 pathway antagonist for the manufacture of a medicament for the prophylactic treatment of chronic obstructive pulmonary disease (COPD).

### BRIEF DESCRIPTION OF THE DRAWINGS

FIG. 1. Effect of cigarette smoke regulation of ABC transporters dependent cholesterol efflux mechanisms on inflammation and tissue destruction in emphysema pathogenesis.

FIG. 2. Potential role of ABC transporters in emphysema development. A. mRNA expression analysis of ABCA1/G1 in lungs of patients with moderate and severe COPD. B. CSE regulation of TNFα, Myd88, MMPs and ABCA1/G1 in macrophages. C. Cholesterol efflux to ApoAI and HDL in macrophages after treatment with CSE for 24h. D. In vitro mRNA expression of TNFα, IL-1β, MMPs and ABCA1/G1 in macrophages isolated from ABCA1 Cre-LysM as compared to WT ABCA1 fl/fl mice. E. Total inflammatory cell counts in BALF of mice (ABCA1 Cre-LysM as compared to WT ABCA1 fl/fl mice) exposed to cigarette smoke for 10 days versus room air exposed control mice. β-Actin was used as housekeeping control both for RT-PCR. Values were considered significant when *p<0.05, **p<0.01, ***p<0.001 vs controls and #p<0.05 vs ABCA1 fl/fl mice exposed to cigarette smoke for 10 days. (F.) In vivo mRNA expression of TNFα, IL-1β, MCP-1, MMP-9 in alveolar macrophages isolated from. ABCA1 Cre-LysM as compared to WT ABCA1 fl/fl mice exposed to cigarette smoke for 10 days versus room air exposed control mice.

FIG. 3. Scheme for lung tissue analysis after chronic cigarette smoke exposure.

FIG. 4. Role of ABC transporter dependent cholesterol efflux mechanisms by LXR agonism or miR-33 antagonism in potential emphysema regression. A. miR-33 expression analysis in macrophages after treatment with CSE and nicotine for 24h. B. Total inflammatory cell counts in BALF of mice exposed to cigarette smoke for 10 days with or without LXR agonist treatment (25 mg/kg IP) versus room air exposed control mice. SnoRNA-32 was used as housekeeping control both for RT-PCR. Values were considered significant when *p<0.05, **p<0.01, ***p<0.001 vs controls and #p<0.05 vs mice exposed to cigarette smoke for 10 days.

FIG. 5. Scheme for ABC transporters modulation in vivo in mice by miR-33 (A.) and LXR agonist (B.) in chronic cigarette smoke exposure.

FIG. 6. Increase in the lung sphingolipid production due to cigarette smoke. Sphingomyelin (A.) and ceramide (B.) levels in the lungs of mice exposed to cigarette smoke for 4 weeks measured by LC/MS/MS in the lungs. (C.) Levels of total ceramide in BAL of mice exposed to cigarette smoke vs room air. Values were considered significant when *p<0.05, ***p<0.001 vs controls and #p<0.05 vs mice exposed to cigarette smoke for 4 weeks. (n=10).

FIG. 7. Role of ABC transporters modulation in sphingolipid turnover in alveolar macrophage and epithelial cells.

FIG. 8. Outline of the timeline of the studies.

FIG. 9. Cigarette smoke induced downregulation of ABC transporter dependent cholesterol efflux in macrophages. (A.) mRNA expression level analysis by RT-PCR of ABCA1 and G1 24h after 5% CSE treatment (n=3). (B.) mRNA expression profile of ABCA1, G1, from thioglycolate-elicited peritoneal macrophages isolated from mice exposed to smoke for 5 days as compared to room air exposed (n=3). (C.) Cholesterol efflux towards ApoAI (25-50 μg) and HDL (25 μg) was measured in thioglycolate-elicited macrophages using tritiated cholesterol (n=3). β-Actin was used as a housekeeping gene control for RT-PCR. Values are presented as statistically significant when *p<0.05, **p<0.01, ***p<0.001 when compared to controls.

FIG. 10. Cigarette smoke induced downregulation of ABC transporter dependent cholesterol efflux in macrophages.

FIG. 11. Correlation of cigarette smoke induced ABC transporters downregulation with inflammation and MMPs in macrophages. (A.) In vitro mRNA expression analysis of TNFα, Myd88, MMPs and ABCA1/G1 in macrophages (n=3) (B.) In vivo mRNA expression analysis of TNFα, Myd88, MMPs and ABCA1/G1 from thioglycolate-elicited peritoneal macrophages isolated from mice exposed to smoke for 5 days as compared to room air exposed (n=3). β-Actin was used as a housekeeping gene control for RT-PCR. Values are presented as statistically significant when *p<0.05, **p<0.01, ***p<0.001 when compared to controls.

FIG. 12. Correlation of cigarette smoke induced ABC transporters downregulation with inflammation and MMPs in macrophages.

FIG. 13. Reestablishment of ABC transporters expressionunder CSE conditions by LXR agonist in macrophages. (A.) In vitro mRNA expression analysis of ABCA1 and G1 in mouse macrophages treated with 5% CSE with or without LXR agonist (T0901317-Cayman) in 3 μM concentration. (B.) Protein analysis of ABCA1 and G1 transporters by Western blot G1 in mouse macrophages isolated from bone marrow of WT (ABCA1 fl/fl mice) and macrophage specific ABCA1 KO (ABCA1 Cre-LysM) treated with 5% CSE with or without LXR agonist (T0901317-Cayman) in 3 μM concentration. β-Actin was used as a housekeeping gene control for RT-PCR and loading control for Western Blot. β-Actin was used as a housekeeping gene control for RT-PCR. Values are presented as statistically significant when **p<0.01, ***p<0.001 when compared to controls and #p<0.05 compared to 5% CSE.

FIG. 14. Effect of LXR dependent ABCA1 reexpression on cigarette smoke induced pro-inflammatory and MMP signaling pathways in macrophages. (A.) Western Blot analysis of JNK phosphorylation (oxidative stress activated MAP kinase), (B.) mRNA expression analysis of TLR4/Myd88 in mouse macrophages treated with 5% CSE with or without LXR agonist (T0901317-Cayman) in 3 μM concentration. (C-E.) mRNA expression analysis of inflammatory cytokines TNFα (C.), IL-1β (D.) and IL-10 (E.) mouse macrophages isolated from bone marrow of WT (ABCA1 fl/fl mice) and macrophage specific ABCA1 KO (ABCA1 Cre-LysM) treated with 5% CSE with or without LXR agonist (T0901317-Cayman) in 3 μM concentration. (F-H.) Analysis of MMP activation mouse macrophages isolated from bone marrow of WT (ABCA1 fl/fl mice) and macrophage specific ABCA1 KO (ABCA1 Cre-LysM) treated with 5% CSE with or without LXR agonist (T0901317-Cayman) in 3 μM concentration. MMP-9 and -13 mRNA expression (F.), MMP-9 mRNA expression with LXR agonist treatment, (H.) MMP-9 activity assayed by gelatin zymography. β-Actin was used as a housekeeping gene control for RT-PCR. Values are presented as statistically significant when *p<0.05, **p<0.01, ***p<0.001 when compared to controls and #p<0.05 compared to 5% CSE.

FIG. 15. Effect of LXR activation on cigarette smoke induced acute pulmonary inflammation and MMP activation. (A.) Total inflammatory cell counts in BALF of mice exposed to cigarette smoke for 10 days with or without LXR agonist treatment (25 mg/kg IP) versus room air exposed control mice (n=4). (B.) Protein concentration of TNFα measured by Luminex cytokines array system, (C.) mRNA analysis of ABCA1, MMP-9 and TNFα, (D.) MMP-9 activity assayed by gelatin zymography in BAL of mice exposed to cigarette smoke for 10 days with or without LXR agonist treatment (25 mg/kg IP) versus room air exposed control mice. (E.) mRNA analysis of ABCA1, MMP-9 and TNFα, (F.) Protein concentration of IL-1β, IL-6, IL-17, IFNγ, MCP-1, TNFα measured by Luminex cytokines array system in the lungs of mice exposed to cigarette smoke for 10 days with or without LXR agonist treatment (25 mg/kg IP) versus room air exposed control mice. β-Actin was used as a housekeeping gene control for RT-PCR. Values are presented as statistically significant when *p<0.05, **p<0.01, ***p<0.001 when compared to controls and #p<0.05 compared to 10 days of cigarette smoke exposure.

FIG. 16. Effect of LXR activation on cigarette smoke induced chronic pulmonary inflammation and MMP activation. (A.) Total inflammatory cell counts in BALF of mice exposed to cigarette smoke for 5 months in AKR/J mice with or without LXR agonist oral treatment in diet (0.015% w/w, approximately 30 mg/kg) versus room air exposed control mice (n=8). (B.) Protein concentration of TNFα measured by Luminex cytokines array system, (C.) Differential BAL macrophages cell count describing regular macrophages and “foamy” like macrophages performed by size differentiation in H&E staining (D.) mRNA analysis of ABCA1 and MMP-9, (E.) MMP-9 activity assayed by gelatin zymography in BAL of mice exposed to cigarette smoke for 5 months in AKR/J mice with or without LXR agonist oral treatment in diet (0.015% w/w, approximately 30 mg/kg) versus room air exposed control mice. (F.) mRNA analysis of ABCA1, ABCG1, MMP-9 and TNFα, (G.) Protein concentration of IL-1β, IL-6, IL-17, IFNγ, MCP-1, TNFα measured by Luminex cytokines array system in the lungs of mice exposed to cigarette smoke for 5 months in AKR/J mice with or without LXR agonist oral treatment in diet (0.015% w/w, approximately 30 mg/kg) versus room air exposed control mice. β-Actin was used as a housekeeping gene control for RT-PCR. Values are presented as statistically significant when *p<0.05, **p<0.01, ***p<0.001 when compared to controls and #p<0.05 compared to 5 months of cigarette smoke exposure.

FIG. 17. Effect of LXR activation on cigarette smoke induced chronic pulmonary inflammation.

FIG. 18. LXR activation significantly improves lung function after chronic cigarette smoke exposure. (A.) Pulmonary compliance [mL/cmH2O], (B.) pulmonary elastance [cmH2O/mL] and pulmonary resistance [cmH2O/mL/sec] were assessed in AKR/J mice exposed to cigarette smoke for 5 months with or without LXR agonist (n=8) oral treatment in diet (0.015% w/w, approximately 30 mg/kg) with use a closed chest model utilizing a flexiVent (SCIREQ) system. Values are presented as statistically significant when *p<0.05, **p<0.01 when compared to 5 months of cigarette smoke exposure.

FIG. 19. LXR activation decrease collagen airway and vessels deposition after chronic cigarette smoke exposure. Lung sections of controls (n=8), smoke exposed for 5 months (n=8) and smoke exposed for 5 months treated with LXR agonist AKR/J mice were stained with Masson Trichrome staining kit (Thermo Scientific) and evaluated for changes in collagen staining around the airways and vessels.

FIG. 20. ABCA1 deficiency in macrophages plays role in pulmonary inflammation acceleration.

FIG. 21. Downregulation of ABC transporters in Chronic Obstructive Pulmonary Disease (COPD) as potential therapeutic target with LXR agonist. (A.) (Left) mRNA expression analysis of ABCA1 G1 in lungs of patients with moderate (after lung volume reduction) and severe COPD (after lung transplantation) and (Right) mRNA expression analysis of ABCA1/G1 in lungs of patients with moderate and severe COPD (n=7). (B.) mRNA analysis of ABCA1, ABCG1, MMP9, (C.) MMP-2 and 9 activity assayed by gelatin zymography in human peripheral blood monocytes differentiated to macrophages in vitro by 3 days treatment with M-CSF (10Ong/m1). After differentiation process human macrophages were treated with 5% CSE with or without LXR agonist (T0901317-Cayman) treatment in 3 μM concentration. β-Actin was used as a housekeeping gene control for RT-PCR. Values are presented as statistically significant when *p<0.05, **p<0.01, ***p<0.001 when compared to controls and #p<0.05 compared Co LXR treated human macrophages.

FIG. 22. Downregulation of ABC transporters in Chronic Obstructive Pulmonary Disease is a therapeutic target with LXR agonist.

FIG. 23. In vivo bioavailability of LXR agonist (T0901317) in the lung and serum after intraperitoneal (IP) and custom made diet administration. (A.) T0901317 LC/MS/MS Chromatogram showing no difference among standard and samples in the peak shape and retension time. (B.) Table representing levels of the administered drug in serum and lungs of AKR/J mice by IP (2 and 24 hours post drug administration) and in custom made diet (Research Diets Inc, 0.015% w/w). The result was normalized by using serum volume and lung tissue weight. No T0901317 was detected in control serum and lung.

FIG. 24. LXR activation significantly improves lung function and structure after chronic cigarette smoke exposure. (A.) Pulmonary compliance [mL/cmH2O], (B.) pulmonary elastance [cmH₂O/mL] and pulmonary resistance [cmH2O/mL/sec] were assessed in AKR/J mice exposed to cigarette smoke for 5 months with or without LXR agonist (n=8) oral treatment in diet (0.015% w/w, approximately 30 mg/kg) with use a closed chest model utilizing a flexiVent (SCIREQ) system. Values are presented as statistically significant when *p<0.05, **p<0.01 when compared to 5 months of cigarette smoke exposure. (C.) Representative H&E sections from lungs of mice exposed to cigarette smoke with or without LXR agonist treatment. (B.) Quantitative analysis of lung destruction as represented by Mean Linear Intercept (MLI). Values are presented as statistically significant when *p<0.05, **p<0.01, ***p<0.001 when compared to controls and #p<0.05 compared to 5 months of cigarette smoke exposure.

FIG. 25. LXR agonist treatment modulates cermmide levels in the lung after chronic cigarette smoke exposure. Mass spectrometry analysis of various ceramide species in Bronchalveolar Lavage (BAL) samples of mice exposed to room air and cigarette smoke with or without LXR agonist treatment. Values are presented as statistically significant when *p<0.05, **p<0.01, ***p<0.001 when compared to controls and #p<0.05 compared to LXR treatment.

FIG. 26. Cigarette smoke induced MMP-9 expression inhibition by other available LXR inhibitors. mRNA analysis of MMP-9 in bone marrow derived macrophages exposed to control media, 5% CSE with and without LXR agonist treatment (A.) DHMCA (10 μM) and (B.) GW3965 (10 μM). Values are presented as statistically significant when *p<0.05, **p<0.01, ***p<0.001 when compared to controls and #p<0.05 compared to LXR agonists treatment

### DETAILED DESCRIPTION OF THE INVENTION

The present invention provides methods for treating a subject afflicted with chronic obstructive pulmonary disease (COPD) which comprises administering to the subject i) a Liver X receptor (LXR) agonist, ii) a miR-33 antagonist, or iii) a TLR4/Myd88 pathway antagonist in an amount that is effective to treat the subject.

In some embodiments, treating the subject comprises improving pulmonary function in the subject.

In some embodiments, treating the subject comprises reducing pulmonary inflammation in the subject.

In some embodiments, the pulmonary inflammation is reduced by about 10, 20, 30, 40, 50, 60, 70, 80, 90, or 100% in the subject compared to the level of pulmonary inflammation when the subject was first administered the LXR agonist, the miR-33 antagonist, or the TLR4/Myd88 pathway antagonist.

In some embodiments, the COPD comprises emphysema.

In some embodiments, treating the subject comprises reducing enphysema in the subject.

In some embodiments, treating the subject comprises slowing or halting the progression of emphysema in the subject.

In some embodiments, treating the subject comprises reversing emphysema in the subject.

In some embodiments, the emphysema is reversed by about 10, 20, 30, 40, 50, 60, 70, 80, 90, or 100% in the subject compared to the level of emphysema when the subject was first administered the LXR agonist, the miR-33 antagonist, or the TLR4/Myd88 pathway antagonist.

In some embodiments, treating the subject comprises reducing obstructive bronchiolitis in the subject.

In some embodiments, treating the subject comprises reducing alveolar or bronchial infiltration of at least one type of inflammatory cell in the subject.

In some embodiments, the at least one type of inflammatory cell comprises macrophages.

In some embodiments, the at least one type of inflammatory cell comprises foamy macrophages.

In some embodiments, the alveolar or bronchial infiltration is reduced by about 10, 20, 30, 40, 50, 60, 70, 80, 90, or 100% in the subject compared to the level of alveolar or bronchial infiltration when the subject was first administered the LXR agonist, the miR-33 antagonist, or the TLR4/Myd88 pathway antagonist.

In some embodiments, treating the subject comprises reducing pulmonary compliance in the subject.

In some embodiments, pulmonary compliance is reduced by about 1, 2, 3, 4, 5, 6, 7, 8, 9, 10, 15, 20 or 25% in the subject compared to the level of pulmonary compliance when the subject was first administered the LXR agonist, the miR-33 antagonist, or the TLR4/Myd88 pathway antagonist.

In some embodiments, the pulmonary compliance is static pulmonary compliance.

In some embodiments, the pulmonary compliance is dynamic compliance.

In some embodiments, treating the subject comprises increasing pulmonary elastance in the subject.

In some embodiments, pulmonary elastance is increased by about 1, 2, 3, 4, 5, 6, 7, 8, 9, 10, 15, 20 or 25% in the subject compared to the level of pulmonary elastance when the subject was first administered the LXR agonist, the miR-33 antagonist, or the TLR4/Myd88 pathway antagonist.

In some embodiments, treating the subject comprises increasing pulmonary resistance in the subject.

In some embodiments, pulmonary resistance is increased by about 1, 2, 3, 4, 5, 6, 7, 8, 9, 10, 15, 20 or 25% in the subject compared to the level of pulmonary resistance when the subject was first administered the LXR agonist, the miR-33 antagonist, or the TLR4/Myd88 pathway antagonist.

In some embodiments, treating the subject comprises reducing mucus hypersecretion in the subject.

In some embodiments, the LXR agonist, the miR-33 antagonist, or the TLR4/Myd88 pathway antagonist decreases the expression or enzymatic activity of at least one matrix metalloproteinase (MMP), in the subject.

In some embodiments, the at least one MMP includes at least one of MMP-1, MMP-9, MMP-12, and MMP-13.

In some embodiments, the at least one MMP includes at least MMP-9.

In some embodiments, the at least one MMP includes at least MMP-13.

In some embodiments, the LXR agonist, the miR-33 antagonist, or the TLR4/Myd88 pathway antagonist decreases the expression of at least one cytokine, in the subject.

In some embodiments, the at least one cytokine includes at least one of TNFα, IL-1, IL-8, IL-13, or IFNγ.

In some embodiments, the at least one cytokine includes at least TNFα.

In some embodiments, the LXR agonist, the miR-33 antagonist, or the TLR4/Myd88 pathway antagonist increases ABCA1 expression in the subject.

In some embodiments, the LXR agonist, the miR-33 antagonist, or the TLR4/Myd88 pathway antagonist increases ABCG1 expression in the subject.

In some embodiments, the LXR agonist, the miR-33 antagonist, or the TLR4/Myd88 pathway antagonist increases the level of sphingosine 1-phosphate (S1P) in the lungs or the serum of the subject.

In some embodiments, the LXR agonist, the miR-33 antagonist, or the TLR4/Myd88 pathway antagonist decreases the level of a ceramide in the lungs or the serum of the subject. In some embodiments, the ceramide is a C14 or a C16 ceramide.

In some embodiments, the LXR agonist, the miR-33 antagonist, or the TLR4/Myd88 pathway antagonist increases the expression of LXR in the subject.

In some embodiments, a LXR agonist is administered to the subject.

In some embodiments, the LXR agonist is an LXRα agonist, an LXRβ agonist, or an LXRα and LxRp agonist.

In some embodiments, the LXR agonist is also a farnesoid X receptor (FXR) agonist.

In some embodiments, the LXR agonist is an organic compound having a molecular weight less than 1000 Daltons, a DNA aptamer, an RNA aptamer, or a polypeptide.

In some embodiments, the LXR agonist is an organic compound having a molecular weight less than 1000 Daltons.

In some embodiments, the LXR agonist is T0901317, GW3965, EXEL2255, N,N-dimethyl-3β-hydroxy-cholenamide (DMHCA), BMS-779788, or an sLXRM, or a pharmaceutically acceptable salt or ester thereof.

In some embodiments, the LXR agonist is other than GW3965, or a pharmaceutically acceptable salt or ester thereof.

In some embodiments, the LXR agonist is a compound that is in a clinical trial or is approved for use in treating atherosclerosis.

In some embodiments, the amount of the LXR agonist administered is less than the amount that is effective for treatment of atherosclerosis.

In some embodiments, a miR-33 antagonist is administered to the subject.

In some embodiments, the miR-33 antagonist is an organic compound having a molecular weight less than 1000 Daltons, a DNA aptamer, an RNA aptamer, an interfering RNA (RNAi) molecule, an antisense oligonucleotide, a ribozyme, or a polypeptide.

In some embodiments, the miR-33 antagonist is an antisense oligonucleotide that targets miR-33.

In some embodiments, the antisense oligonucleotide that targets miR-33 is a morpholino oligomer.

In some embodiments, the antisense oligonucleotide has nucleotides in the sequence: TGC AAT GCA ACT ACA ATG CAC.

In some embodiments, a TLR4/Myd88 pathway antagonist is administered to the subject.

In some embodiments, the TLR4/Myd88 pathway antagonist is an organic compound having a molecular weight less than 1000 Daltons, a DNA aptamer, an RNA aptamer, an interfering RNA (RNAi) molecule, an antisense oligonucleotide, a ribozyme, a polypeptide, or an antibody.

In some embodiments, the TLR4 Myd88 pathway antagonist is an anti-TLR4 antibody.

In some embodiments, the TLR4/Myd88 pathway antagonist is an organic compound having a molecular weight less than 1000 Daltons.

In some embodiments, the TLR4/Myd88 pathway antagonist is an IRAK inhibitor.

In some embodiments, the TLR4/Myd88 pathway antagonist is a peptide.

In some embodiments, the peptide is a Myd88 blocking peptide.

In some embodiments, the TLR4/Myd88 pathway antagonist is an interfering RNA (RNAi) molecule, an antisense oligonucleotide, or a ribozyme, that i) targets TLR4-encoding mRNA and is capable of reducing TLR4 expression or ii) targets Myd88-encoding mRNA and is capable of reducing Myd88 expression.

In some embodiments, two or more of the LXR agonise, the miR-33 antagonist, or the TLR4/Myd88 pathway antagonist are administered to the subject.

Some embodiments further comprise administering an additional compound to the subject, each of the LXR agonist, the miR-33 antagonist, or the TLR4/Myd88 pathway antagonist and the additional compound being administered in an amount such that, when administered in combination, the administration of the LXR agonist, the miR-33 antagonist, or the TLR4/Myd88 pathway antagonist and the additional compound is effective to treat the subject.

In some embodiments, the additional compound is a steroid.

In some embodiments, the steroid is a glucocorticosteroid.

In some embodiments, the additional compound is other than a steroid.

In some embodiments, the additional compound is an MMP inhibitor.

In some embodiments, the additional compound lowers plasma or liver triglycerides in the subject.

In some embodiments, the additional compound is a bronchodilator.

In some embodiments, the LXR agonist, the miR-33 antagonist, or the TLR4/Myd88 pathway antagonist is administered as a monotherapy.

The present invention provides methods for prophylactically treating a subject for chronic obstructive pulmonary disease (COPD) which comprises administering to the subject i) a Liver X receptor (LXR) agonist, ii) a miR-33 antagonist, or iii) a TLR4/Myd88 pathway antagonist in an amount that is effective to treat the subject.

In some embodiments, the subject is a mammalian subject.

In some embodiments, the subject is a human subject.

In some embodiments, the subject has a substantially healthy cardiovascular system.

In some embodiments, the subject has hypercholesterolemia.

In some embodiments, if the subject is receiving treatment for a disease other than COPD then the disease other than COPD is other than atherosclerosis.

In some embodiments, there is abnormal cholesterol efflux in the lungs of the subject.

In some embodiments, there is abnormal cholesterol homeostasis in the lungs of the subject.

In some embodiments, the subject is or has been a cigarette smoker.

In some embodiments, the COPD is caused by chronic cigarette smoking.

The present invention provides methods for identifying whether a subject afflicted with chronic obstructive pulmonary disease (COPD) is responding to treatment for COPD comprising
i) periodically obtaining biological samples from the subject;
ii) assaying whether the level of a ceramide has increased or decreased in the biological samples over a period of time, and
iii) identifying the subject as responding to treatment if the level of the ceramide has decreased over the period of time.

The present invention provides methods for identifying whether a subject afflicted with chronic obstructive pulmonary disease (COPD) is responding to treatment for COPD comprising
i) periodically obtaining biological samples from the subject;
ii) assaying whether the level of sphingosine 1-phosphate (S1P) has increased or decreased in the biological samples over a period of time, and
iii) identifying the subject as responding to treatment if the level of S1P has increased over the period of time.

The present invention provides methods for determining whether chronic obstructive pulmonary disease (COPD) is progressing in a subject afflicted with COPD comprising
i) periodically obtaining biological samples from the subject;
ii) assaying whether the level of a ceramide has increased or decreased in the biological samples over a period of time, and
iii) identifying the COPD as progressing in the subject if the level of the ceramide has decreased over the period of time.

The present invention provides methods for determining whether chronic obstructive pulmonary disease (COPD) is progressing in a subject afflicted with COPD comprising
i) periodically obtaining biological samples from the subject;
ii) assaying whether the level of sphingosine 1-phosphate (S1P) has increased or decreased in the biological samples over a period of time, and
iii) identifying the COPD as progressing in the subject if the level of sphingosine 1-phosphate (S1P) has increased over the period of time.

In some embodiments, the subject is treated in accordance with a method of the present invention if in step iii) the COPD is identified as progressing in the subject.

In some embodiments, the subject. is receiving treatment comprising i) a Liver X receptor (LXR) agonist, ii) a miR-33 antagonist, or iii) a TLR4/Myd88 pathway antagonist.

Some embodiments comprise continuing to treat the subject with the Liver X receptor (LXR) agonist, the miR-33 antagonist, or the TLR4/Myd88 pathway antagonist if in step iii) COPD is identified as progressing in the subject.

In some embodiments, the period of time is about 1, 2, 3, 4, 5, 6, 7, 8, 9, 10, 15, 20, 25, 30, 35, 40, 45, or 50 weeks.

In some embodiments, the biological sample is serum.

In some embodiments, the biological sample is bronchoalveolar lavage fluid.

The present invention provides compositions for use in treating a subject afflicted with chronic obstructive pulmonary disease (COPD) which comprises i) a Liver X receptor (LXR) agonist, ii) a miR-33 antagonist, or iii) a TLR4/Myd88 pathway antagonist.

The present invention provides compositions for use in prophylactically treating a subject for chronic obstructive pulmonary disease (COPD) which comprises i) a Liver X receptor (LXR) agonist, ii) a miR-33 antagonist, or iii) a TLR4/Myd88 pathway antagonist.

Aspects of the present invention relate to the use of i) a Liver X receptor (LXR) agonist, ii) a miR-33 antagonist, or iii) a TLR4/Myd88 pathway antagonist for the manufacture of a medicament for the treatment of chronic obstructive pulmonary disease (COPD).

Aspects of the present invention relate to the use of i) a Liver X receptor (LXR) agonist, ii) a miR-33 antagonist, or iii) a TLR4/Myd88 pathway antagonist for the manufacture of a medicament for the prophylactic treatment of chronic obstructive pulmonary disease (COPD).

Aspects of the present invention relate to the use of LXR agonists can be used treat emphysema and COPD. In some embodiments, LXR agonists are combined with other drugs to enhance efficacy.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, “about” in the context of a numerical value or range means ±10% of the numerical value or range recited or claimed, unless the context requires a more limited range.

As used herein, “monotherapy” means a therapy that is administered to treat a disease, such as COPD, without any other therapy that is used specifically to treat the disease. A monotherapy for treating COPD may optionally be combined with another treatment that is used to ameliorate a symptom of COPD, but may not be combined with any other therapy directed against COPD itself. For example, administering an LXR agonist as a monotherapy means administering the LXR agonist without a glucocorticosterord. However, in some embodiments of the invention, agents that are not directed against COPD, for example pain killers, may be administered concurrently or simultaneously with the LXR agonist monotherapy.

As used herein, “a subject afflicted with” a disease, e.g. COPD, means a human patient who was been affirmatively diagnosed to have the disease.

As used herein, “effective” when referring to an amount of a compound or compounds refers to the quantity of the compound or compounds that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the physical condition of the patient, the type of subject being treated, the duration of the treatment, the nature of concurrent therapy (if any), and the specific formulations employed and the structure of the compounds or its derivatives.

The term “LXR” (liver X receptor) or “LXR receptor” includes all subtypes of this receptor. Specifically LXR includes LXRα and LXRβ. LXRα has been referred to under a variety of names such as LXRU, LXRα, LXR, RLD-1, NR1H3. It encompasses any polypeptide encoded by a gene with substantial sequence identity to GenBank accession number U22662. Similarly, LXRβ included any polypeptide encoded by a gene referred to as LXRb, LXRP, LXRbeta, NER, NER1, UR, OR-1, RIP 15, NR1H2 or a gene with substantial sequence identity to GenBank accession number U07132.

LXR Agonists

There are many LXR agonists that are suitable for practicing methods of the present invention. They can be known agents that activate LXR receptor, e.g., GW3965, or other commercially available compounds such as F3-MethylAA (from Merck; see Menke et al., Endocrinology 143: 2548-58, 2002) and T0901317 (Tularik, Calif.). They can also be novel LXR agonists to be screened for as described below. As detailed below, the LXR agonists suitable for the present invention can be polypeptides, peptides, small molecules, or other agents, The LXR agonists ran be agonists for LXR of human as well as other subjects.

A great number of LXR agonists have been described in the art. Examples of small molecule LXR agonists include the well known oxysterols and related compounds (Janowski et al., Nature 383: 728-31, 1996); T0901317 and T0314407 (Schultz et al., Genes Dev 14: 2831-8, 2000); 24(S)-hydroxycholesterol, and 22(R)-hydroxycholesterol (Janowski et al., Nature 383: 728-731, 1996); and 24,25-epoxycholesterol (U.S. Pat. No. 6,316,503). Exemplary polypeptide agonists of LXR have also been disclosed in the art, e.g., WO 02/077229. Additional LXR agonists have been described in the art, e.g., in U.S. Pat. No. 6,316,503; Collins et al., J Med. Chem. 45: 1963-6, 2002; Joseph et al., Proc Natl Acad Sci USA 99: 7604-9, 2002; Menke et al., Endocrinology 143: 2548-58, 2002; Schultz et al., Genes Dev. 14: 2831-8, 2000; and Schmidt et al., Mol Cell Endocrinol. 155: 51-60, 1999.

Many LXR agonists are effective in activating both LXRα and LXRβ (e.g., GW3965 as described in Collins et al., J Med. Chem. 45: 1963-6, 2002). Some LXR agonists activate LXRα and LXRβ under different conditions. For example, 6-alpha-hydroxylated bile acids are agonists of LXRα, but also activate LXRβ at higher concentrations (Song et al., Steroids 65: 423-7, 2000). Some LXR agonists act exclusively on LXRα, while some others activate only LXRβ. For example, introduction of an oxygen on the sterol B-ring of oxysterol results in a ligand with LXRa-subtype selectivity (Janowski et al., Proc Natl Acad Sci USA 96: 266-71, 1999). Using ligand-dependent transcription assays, it was found that 5-tetradecyloxy-2-furancarboxylic acid (TOFA) and hydroxycholesterol transactivates chimeric receptors composed of the glucocorticoid receptor DNA binding domain and the ligand binding regions of LXRβ, PPARα, and PPARβ receptors (Schmidt et al., Mol Cell Endocrinol. 155: 51-60, 1999).

LXR agonists can also be obtained from derivatives of known polypeptide agonists of the LXR receptor. They can be produced by a variety of art known techniques. For example, specific oligopeptides (e.g., 10-25 amino acid residues) spanning a known polypeptida agonise of LXR can be synthesized (e.g., chemically or recombinantly) and tested for their ability to activate an LXR receptor. The LXR agonist fragments can be synthesized using standard techniques such as those described in Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers are commercially available, e.g., from Advanced ChemTech Model 396; Milligen/Biosearch 9600. Alternatively, such LXR agonists can be produced by digestion of native or recombinantly produced polypeptide agonists of LXR using a protease, e.g., trypsin, thermolysin, chymotrypsin, or pepsin. Computer analysis (using commercially available software, e.g. MacVector, Omega, PCGene, Molecular Simulation, Inc.) can be used to identify proteolytic cleavage sites.

The polypeptide or peptide agonists for use in methods of the present invention are preferably isolated and substantially free of cellular material or other contaminating proteins from the cell or tissue source from which the LXR agonist is derived, or substantially free from chemical precursors or other chemicals when chemically synthesized. The proteolytic or synthetic polypeptide agonists or their fragments can comprise as many amino acid residues as are necessary to activate LXR receptor activity, and can comprise at least 5, 10, 15, 20, 25, 30, 35, 40, 45, 50, or more amino acids in length.

Other than known compounds and polypeptides that activate the LXR receptor, LXR agonists can also be obtained by screening test agents (e.g., compound libraries) to identify novel LXR agonists that bind to and/or activate LXR receptor activities. To screen for such novel LXR agonists, a human LXR or LXR of other animals can be employed in a proper assay system. Polynucleotide and amino acid sequences of the LXR receptors are known and described in the art. Their structures and functional organizations, including their ligand binding domains, have also been characterized. See, e.g., Apfel et al., Mol Cell Biol 14: 7025-7035, 1994; Willy et al., Genes Dev 9: 1033-1045, 1995; Song et al., Proc Natl Acad Sci USA 91: 10809-10813, 1994; Shinar et al., Gene 147: 273-276, 1994; Teboul at al., Proc Natl Acad Sci USA 92: 2096-2100, 1995; and Seol et al., Mol Endocrinol 9: 72-85, 1995.

Aspects of the invention relate to agonists that can activate either LXRα or LXRβ, or both LXRα and LXRβ. In addition, instead of the full length LXR molecule, some of the screen assays can employ an LXR polypeptide that comprises a fragment of an LXR molecule. For example, the two functional domains of the LXR receptor, the N-terminal DNA binding domain (DBD) and the C-terminal ligand-binding domain (LBD), mediate the transcriptional activation function of nuclear receptors. An LXR polypeptide containing any of these domains can be used in screening for novel LXR agonists.

A number of assay systems can be employed to screen test agents for agonists of an LXR receptor. As detailed below, test agents can be screened for direct binding to an LXR polypeptide or a fragment thereof (e.g., its ligand binding domain). Alternatively or additionally, potential LXR agonists can be examined for ability to activate LXR receptor pathway or stimulate other biological activities of the LXR receptor. Either an in vitro assay system or a cell-based assay system can be used in the screening.

Selectivity of potential LXR agonists for different receptors can be tested using methods well known in the art, e.g., the LXR radioligand competition scintillation proximity assays described in, e.g., WO 01/41704.

Test agents that can be screened for novel LXR agonists include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position.

Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small organic molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of an LXR receptor. A number of assays are available for such screening, e.g., as described in Schultz (1998) Bioorg Med Chem Lett 8: 2409-2414; Weller (1997) Mol. Divers. 3: 61-70; Fernandes (1998) Curr Opin Chem Biol 2: 597-603; and Sittampalam (1997) Curr Opin Chem Biol 1: 384-91.

Potential LXR agonists can also be identified based on rational design. For example, Janowski et al. (Proc Natl Acad Sci USA 96: 266-71, 1999) disclosed structural requirements of ligands for LXRalpha and LXRbeta. It was shown that position-specific monooxidation of the sterol side chain of oxysterol is requisite for LXR high-affinity binding and activation. Enhanced binding and activation can also be achieved through the use of 24-oxo ligands that act as hydrogen bond acceptors in the side chain. In addition, introduction of an oxygen on the sterol B-ring results in a ligand with LXRalpha-subtype selectivity.

Libraries of test agents to be screened with the claimed methods can also be generated based on structural studies of the LXR receptors, their fragments or analogs. Such structural studies allow the identification of test agents that are more likely to bind to the LXR receptor. The three-dimensional structure of an LXR receptor can be studied in a number of ways, e.g., crystal structure and molecular modeling. Methods of studying protein structures using xray crystallography are well known in the literature. See Physical Bio-chemistry, Van Holde, K. E. (Prentice-Hall, NJ. 1971), pp, 221-239, and Physical Chemistry with Applications to the Life Sciences, D. Eisenberg & D. C. Crothers (Benjamin Cummings, Menlo Park 1979). Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, N.J. 1972), and NMR of Proteins and Nucleic Acids, K. Wuthrich (Wiley-Interscience, New York 1986).

In some screening assays, binding of a test agent to an LXR or an LXR polypeptide containing its ligand binding domain is determined. Binding of test agents (e.g., polypeptides) to the LXR polypeptide can be assayed by a number of methods including, e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13: 115-122, 1995; Ecker et al., Bio/Technology 13: 351-360, 1995; and Hodgson, Bio/Technology 10: 973-980, 1992. The test agent can be identified by detecting a direct binding to the LXR polypeptide, e.g., co-immunoprecipitation with the LXR polypeptide by an antibody directed to the LXR polypeptide. The test agent can also be identified by detecting a signal that indicates that the agent binds to the LXR polypeptide, e.g., fluorescence quenching.

Competition assays provide a suitable format for identifying test agents (e.g., peptides or small molecule compounds) that specifically bind to an LXR polypeptide. In such formats, test agents are screened in competition with a compound already known to bind to the LXR polypeptide. The known binding compound can be a synthetic compound. It can also be an antibody, which specifically recognizes the LXR polypeptide, e.g., a monoclonal antibody directed against the LXR polypeptide. If the test agent inhibits binding of the compound known to bind the LXR polypaptide, then the test agent also binds the LXR polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9: 242-253 (1983)); solid phase direct biotin-avidin EIA (see Kirkland et al., J. Immunol. 137: 3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using 125I label (see Morel et al., Mol. Immunol. 25(1):7-15 (1988)); solid phase direct biotin-avidin EIA (Cheung et al., Virology 176: 546-552 (1990)); and direct labeled RIA (Moldenhauer et al., Scand. J. Immunol. 32: 77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an LXR polypeptide or its fragments onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the LXR polypeptide, the test agents are bound to the solid matrix and the LXR polypeptide molecule is then added.

Soluble assays include some of the combinatory libraries screening methods described above. Under the soluble assay formats, neither the test agents nor the LXR polypeptide are bound to a solid support. Binding of an LXR polypeptide or fragment thereof to a test agent can be determined by, e.g., changes in fluorescence of either the LXR polypeptide or the test agents, or both. Fluorescence may be intrinsic or conferred by labeling either component with a fluorophor.

In some binding assays, either the LXR polypeptide, the test agent, or a third molecule (e.g., an antibody against the LXR polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., 125I, 32P, 35S) or a chemiluminescent or fluorescent group. Similarly, the detectable group can be a substrate, cofactor, inhibitor or affinity ligand.

Binding of a test agent to LXR can also be tested indirectly with a cell-based assay. For example, a DNA-binding domain of the nonreceptor transcription factor GAL4 can be fused to the ligand-binding domain of LXR (e.g., LXRalpha). The resultant construct is introduced into a host cell (e.g., the 293 cells) together with a reporter construct (e.g., a UAS-containing luciferase reporter construct). The transfected cells are then treated with libraries of test agents, and reporter polypeptide activity (e.g., luciferase activity) is measured. Effects of individual test agents on the reporter polypeptide activity are evaluated relative to a control (i.e., when no test compound is present).

The cell-free ligand sensing assay (LiSA) can also be employed to identify novel LXR agonists. It can be performed as described in the art, e.g., Collins et al., J Med. Chem. 45: 1963-6, 2002; and Spencer et al., J. Med. Chem. 44: 886-97, 2001. This assay measures the ligand-dependent recruitment of a peptide from the steroid receptor coactivator 1 (SRC1) to the nuclear receptor. With this assay (LiSA), the structural requirements for activation of the LXR receptor by test agents can be studied.

Other than or in addition to detecting a direct binding of a test agent to an LXR polypeptide, potential LXR agonists for use in the methods of the present invention can also be examined for ability to activate other bioactivities or cellular activities of the LXR receptor. Test agents which activate LXR receptor can be identified by monitoring their effects on a number of LXR cellular activities. LXR cellular activities include any activity mediated by activated LXR receptor (e.g., transcriptional regulation of a target gene). For example, LXR trans-activate expression of a number of target genes (e.g., ABCA1), inhibit fibroblast differentiation to adipocytes, modulate the production of muscle-specific enzymes, e.g., creatine kinase, modulate glucose uptake by cells, and stimulate myoblast cell proliferation. The degree to which a test agent activates an LXR receptor can be identified by testing for the ability of the agent to enhance such LXR activities.

Thus, a novel LXR agonist can be identified by identifying a test agent that enhances expression of an LXR target gene (e.g., ABCA1, ABCG1, SREBP1, or the cholesterol 7-hydroxylase gene). Methods for identifying test agents that induce an LXR target gene expression (e.g., increasing ABCA1 mRNA levels) have been disclosed in the art, e.g., Menke et al., Endocrinology 143: 2548-58, 2002; Sparrow et al., J. Biol. Chem. 277: 10021-7, 2002; and Murthy et al., J Lipid Res. 43: 1054-64, 2002.

Other than monitoring LXR target gene expression, LXR agonists can also be identified by examining other cellular activities stimulated by the LXR pathway. For example, LXR agonists modulate the protein level and hence activity of a muscle-specific enzyme, creatine kinase. Therefore, F. egonis ts can be screened by examining test agents for ability to modulate creatine kinase activity, e.g., as described in Somjen et al., J Steroid Biochem Mol Biol 62: 401-8, 1997. The assay can be performed in a cell line, e.g., the mouse skeletal myoblast cell line or a primary chick myoblast cell line. Effects of test compounds on creatine kinase activity in the cultured cells can be measured in the cell lysates using a commercially available kit (available by Sigma, St Louis, Mo., USA).

Modulation of other cellular bioactivities of the LXR receptor can also be detected using methods well known and routinely practiced in the art. For example, the test agent can be assayed for their activities in increasing cholesterol efflux from cells such as macrophages (Menke et al., Endocrinology 143: 2548-58, 2002; and Sparrow et al., J. Biol. Chem. 277: 10021-7, 2002). Other assays include ligand-dependent transcription assays (Schmidt et al., Mol Cell Endocrinol 155: 51-60, 1999), methods for measuring the ability of LXR agonists to interfere with the differentiation process of pre-adipocytes (fibroblasts) to adipocytes (Plaas et al., Biosci Rep 1: 207-16, 1981; Hiragun et al., J Cell Physiol 134: 124-30, 1988; and Liao et al., 3 Biol Chem 270: 12123-32, 1995), or the ability to stimulate myoblast cell proliferation (Konishi et al., Biochemistry 28: 8872-7, 1989; and Austin et al., J Neurol Sci 101: 193-7, 1991). As a control, all these assays can include measurements before and after the test agent is added to the assay system.

LXR agonists are effective for treatment of murine models of atherosclerosis, diabetes, anti-inflammation, and Alzheimer's disease. Treatment with LXR agonists (e.g., hypocholamide, T0901317, GW3965, or N,N-dimethyl-3betahydroxy-cholenamide (DMHCA)) lowers the cholesterol level in serum and liver and inhibits the development of atherosclerosis in murine disease models.

Non-limiting examples of commercially available LXR agonists include:
T0901317 (Cayman Chemical Company, Ann arbor, Mich., USA)
GW3965 (Sigma Aldrich, St. Louis, Mo., USA)
EXEL2255 (Exelixis Inc., So, San Francisco, Calif., USA)
sLXRMs (Phenex Pharmaceuticals AG, Ludwigshafen, Germany)
BMS-779788 (Bristol - Myers Squibb Company, New York, N.Y., USA)

T0901317 (Eli Lilly & Co) is a synthetic liver X receptor agonist that decreases blood glucose levels and improves insulin sensitivity by downregulation of expression of genes important for liver gluconeogenesis (phosphoenolpyruvate carboxykinase and glucose 6-phosphate dehydrogenase) and upregulation of the glucose transporter GLUT4 in adipose tissue. T0901317 is indicated for the treatment of type II diabetes. The ability of the liver X receptor (LXRα (NR1H3) and LXRβ (NR1H2)) agonist, T0901317, to activate the farnesoid X receptor (FXR (NR4H4)) has been characterized. Although T0901317 is a much more potent activator of LXR than FXR, this ligand actually activates FXR more potently than a natural bile acid FXR ligand, chenodeoxycholic acid. The structure of T0901317 is:

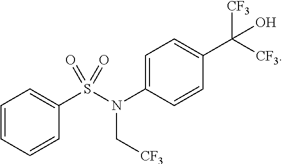 [SMILES: O=S(=O)(c1ccccc1)N(CC(F)(F)F)c1ccc(C(O)(C(F)(F)F)C(F)(F)F)cc1]

The CAS Registry number for T0901317 is 293754-55-9. T0901317 is also known as T131, TO 901317 and N-(2,2,2-trifluoroethyl)-N-[4-[2,2,2-trifluoro-1-hydroxy-1-(trifluoromethyl)ethyl]phenyl]-benzenesulfonamide. TO 901317 is described in Quinet et a (2004) “Gene-selective modulation by a synthetic oxysterol ligand of the liver X receptor” J. Lipid Res. 45 1929-1942, the entire content which is hereby incorporated herein in its entirety.

GW3965 is a liver X receptor agonist that is commercially available as GW3965 hydrochloride (Sigma Aldrich, St. Louis, Mo., USA). The structure of GW3965 hydrochloride is:

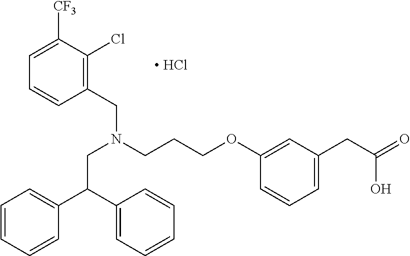 [SMILES: O=C(O)Cc1cccc(OCCCN(Cc2cccc(C(F)(F)F)c2Cl)CC(c2ccccc2)c2ccccc2)c1 ; "· HCl"]

The CAS Registry number for GW3965 hydrochloride is 405911-17-3. GW3965 hydrochloride is also known as 3-[3-[N-(2-Chloro-3-trifluoromethylbenzyl)-(2,2-diphenylethyl)amino]propyloxy]phenylacetic acid hydrochloride. GW3965 is described in Quinet et al (2004) “Gene-selective modulation by a synthetic oxysterol ligand of the liver X receptor” J. Lipid Res. 45 1929-1942, the entire content which is hereby incorporated herein in its entirety.

EXEL2255 (Exelixis Inc., So. San Francisco, Calif., USA) is phase I investigation drug. It is a liver X receptor agonist, a nuclear hormone receptor that regulates cellular cholesterol outflow from the macrophage Co the blood and ultimately to the liver where cholesterol is removed from the body through the process called reverse cholesterol transport.

sLXRMs (Phenex Pharmaceuticals AG, Ludwigshafen, Germany) are nonsteroidal selective LXR (Liver X Receptor) modulators (sLXRMs) with submicromolar potencies that activate cholesterol efflux in human macrophages via induction of ABC transporters (ABCA1). They are being developed for the treatment of atherosclerosis. sLXRMs are in the Pre-Clinical phase.

BMS-779788 (Bristol-Myers Squibb Company, New York, N.Y., USA) is a LXR agonist that is indicated for the treatment of atherosclerosis. Bristol-Myers Squibb Company completed a Phase I placebo-controlled, ascending, multiple-dose study to evaluate the safety, pharmacokinetics and pharmacodynamics of BMS-779788 in healthy subjects. The study was interventional, randomized, safety, parallel assignment, double blind study to evaluate the safety and tolerability of multiple oral doses of BMS-779788 in healthy subjects. The study was initiated in February 2009.

N,N-dimethyl-3β-hydroxy-cholenamide (DMHCA) is a steroidal liver X receptor agonist. The structure of DMHCA is:

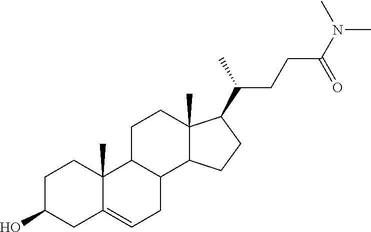 [SMILES: C[C@H](CCC(=O)N(C)C)[C@H]1CCC2C3CC=C4C[C@@H](O)CC[C@]4(C)C3CC[C@@]21C]

DMHCA is described in Kratzer et al (2009) “Synthetic LXR agonist attenuates plaque formation in apoE−/− mice without inducing liver steatosis and hypertriglyceridemia” J Lipid Res. 50(2):312-26 and Quinet et al (2004) “Gene-selective modulation by a synthetic oxysterol ligand of the liver X receptor” J. Lipid Res. 45 1929-1942. 96:266-271, the entire contents of each of which are hereby incorporated herein in their entireties.

Additional non-limiting examples of liver X receptor agonists are described in U.S. Patent No. 7,012,069, issued Mar. 14, 2006; U.S. Pat. No. 7,495,004, issued Feb. 24, 2009; U.S. Pat. No. 8,030,335, issued Oct. 4, 2011; U.S. Pat. No. 8,569,352, issued Oct. 29, 2013; and U.S. Patent Application Publication No. 2011/0059932, published Mar. 10, 2011, the entire contents of each of which are hereby incorporated herein by reference.

Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used Antisense oligonucleotide molecules can be provided in a DNA construct and introduced into a cell as described above to decrease the level of target gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester intemucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of gene expression can be obtained by designing antisense oligonucleotides which will form duplexes to the control, 5′, or regulatory regions of the gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (Nicholls et al., 1993, J Immunol Meth 165:81-91). An antisense oligonucleotide also can be designed to block translation of mRNA by preventing the transcript from binding to ribosomes.

Precise complementarity is not required for successful complex formation between an antisense oligonucleotide and the complementary sequence of a target polynucleotide. Antisense oligonucleotides which comprise, for example, 1, 2, 3, 4, or 5 or more stretches of contiguous nucleotides which are precisely complementary to a target polynucleotide, each separated by a stretch of contiguous nucleotides which are not complementary to adjacent nucleotides, can provide sufficient targeting specificity for a target mRNA. Preferably, each stretch of complementary contiguous nucleotides is at least 4, 5, 6, 7, 8, 9, 10, 11, 12, 13, 14, 15, 16, 17, 18, 19, 20, 21, 22, 23, 24, 25, or more nucleotides in length. Noncomplementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular target polynucleotide sequence. Antisense oligonucleotides can be modified without affecting their ability to hybridize to a target polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art.

Ribozymes

Ribozymes are RNA molecules with catalytic activity (Uhlmann et al., 1987, Tetrahedron. Lett. 215, 3539-3542). Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a polynucleotide can be used to generate ribozymes which will specifically bind to rnRNA transcribed from the polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease target gene expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or VAS element, and a transcriptional teminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

RNA Interference

Some embodiments the invention relate to an interfering RNA (RNAi) molecule. RNAi involves mRNA degradation. The use of RNAi has been described in Fire et al., 1998, Carthew at aJ, 2001, and Elbashir et al., 2001, the contents of which are incorporated herein by reference.

Interfering RNA or small inhibitory RNA (RNAi) molecules include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs can be perfect (i.e., fully complementary) or imperfect, including bulges in the duplex region.

Interfering RNA molecules encoded by recombinase-dependent transgenes of the invention can be based on existing shRNA, siRNA, piwi-interacting RNA (piRNA), micro RNA (miRNA), double-stranded RNA (dsRNA), antisense RNA, or any other RNA species that can be cleaved inside a cell to form interfering RNAs, with compatible modifications described herein.

As used herein, a “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a shRNA may form a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stern portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA or a mature shRNA. For example, an RNAi construct is an expression vector capable of giving rise to a siRNA or a mature shRNA in vivo. Non-limiting examples of vectors that may be used in accordance with the present invention are described herein and will be well known to a person having ordinary skill in the art. Exemplary methods of making and delivering long or short RNAi constructs can be found, for example, in WO01/68836 and WO01/75164.

Use of RNAi

RNAi is a powerful tool for in vitro and in vivo studies of gene function in mammalian cells and for therapy in both human and veterinary contexts. Inhibition of a target gene is sequence-specific in that gene sequences corresponding to a portion of the RNAi sequence, and the target gene itself, are specifically targeted for genetic inhibition. Multiple mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC to silence target mRNA expression.

Another mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982) Conventional shRNAs, which mimic pre-miRNA, are transcribed by RNA Polymerase II or III as single-stranded molecules that form stem-loop structures. Once produced, they exit the nucleus, are cleaved by DICER, and enter the RISC as siRNAs.

Another mechanism is identical to the second mechanism, except that the shRNA is modeled on primary miRNA (shRNAmir), rather than pre-miRNA transcripts (Fewell et al., 2006). An example is the miR-30 miRNA construct. The use of this transcript produces a more physiological shRNA that reduces toxic effects. The shRNAmir is first cleaved to produce shRNA, and then cleaved again by DICER to produce siRNA. The siRNA is then incorporated into the RISC for target mRNA degradation. However, aspects of the present invention relate to RNAi molecules that do not require DICER cleavage. See, e.g., U.S. Pat. No. 8,273,871, the entire contents of which are incorporated herein by reference.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC.

It is not necessary that there be perfect correspondence of the sequences, but the correspondence must be sufficient to enable the RNA to direct RNAi inhibition by cleavage or blocking expression of the target mRNA. In preferred RNA molecules, the number of nucleotides which is complementary to a target sequence is 16 to 29, 18 to 23, or 21-23, or 15, 16, 17, 18, 19, 20, 21, 22, 23, 24, or 25.

Isolated RNA molecules can mediate RNAi. That is, the isolated RNA molecules of the present invention mediate degradation or block expression of mRNA that is the transcriptional product of the gene. For convenience, such mRNA may also be referred to herein as mRNA to be degraded. The terms RNA, RNA molecule(s), RNA segment(s) and RNA fragment(s) may be used interchangeably to refer to RNA that mediates RNA interference. These terms include double-stranded RNA, small interfering RNA (siRNA), hairpin RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules are referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In one embodiment, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA.

In some embodiments, an RNAi molecule of the invention is introduced into a mammalian cell in an amount sufficient to attenuate target gene expression in a sequence specific manner. The RNAi molecules of the invention can be introduced into the cell directly, or can be nompisxed with cationic- lipids, packaged within liposomes, or otherwise delivered to the cell. In certain embodiments the RNAi molecule can be a synthetic RNAi molecule, including RNAi molecules incorporating modified nucleotides, such as those with chemical modifications to the 2′-OH group in the ribose sugar backbone, such as 2′-O-methyl (2′OMe), 2′-fluoro (2′F) substitutions, and those containing 2′OMe, or 2′F, or 2′-deoxy, or “locked nucleic acid” (LNA) modifications. In some embodiments, an RNAi molecule of the invention contains modified nucleotides that increase the stability or half-life of the RNAi molecule in vivo and/or in vitro. Alternatively, the RNAi molecule can comprise one or more aptamers, which interact(s) with a target of interest to form an aptamer:target complex. The aptamer can be at the 5′ or the 3′ end of the RNAi molecule. Aptamers can be developed through the SELEX screening process and chemically synthesized. An aptamer is generally chosen to preferentially bind to a target. Suitable targets include small organic molecules, polynucleotides, polypeptides, and proteins. Proteins can be cell surface proteins, extracellular proteins, membrane proteins, or serum proteins, such as albumin. Such target molecules may be internalized by a cell, thus effecting cellular uptake of the shRNA. Other potential targets include organelles, viruses, and cells.

As noted above, the RNA molecules of the present invention in general comprise an RNA portion and some additional portion, for example a deoxyribonucleotide portion. The total number of nucleotides in the RNA molecule is suitably less than in order to be effective mediators of RNAi. In preferred RNA molecules, the number of nucleotides is 16 to 29, more preferably 18 to 23, and most preferably 21-23.

Dosage Forms and Administration

Ester derivatives of compounds used in the subject invention may be generated from a carboxylic acid group in accordance with the present invention using standard esterification reactions and methods readily available and known to those having ordinary skill in the art of chemical synthesis. Ester derivatives may serve as pro-drugs that can be converted into compounds of the invention by serum esterases.

Compounds used in the methods of the present invention may be prepared by techniques well know in organic synthesis and familiar to a practitioner ordinarily skilled in the art. However, these may not be the only means by which to synthesize or obtain the desired compounds.

Compounds used in the methods of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

In some embodiments, a compound may be in a salt form. As used herein, a “salt” is a salt of the instant compound which has been modified by making acid or base salts of the compounds. In the case of the use of compounds of the invention for treatment of COPD, the salt is pharmaceutically acceptable. Examples of pharmaceutically acceptable salts include, but are not limited to, mineral or organic acid salts of basic residues such as amines. The term “pharmaceutically acceptable salt” in this respect, refers to the relatively non-toxic, inorganic and organic base addition salts of compounds of the invention. These salts can be prepared in situ during the final isolation and purification of a compound, or by separately reacting a purified compound in its free acid form with a suitable organic or inorganic base, and isolating the salt thus formed.

The compounds used in some embodiments of the present invention can be administered in a pharmaceutically acceptable carrier. As used herein, a “pharmaceutically acceptable carrier” is a pharmaceutically acceptable solvent, suspending anent or vehicle, for delivering the compounds to the subject. The carrier may be liquid or solid and is selected with the planned manner of administration in mind. Liposomes are also a pharmaceutically acceptable carrier. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

“Administering” compounds in embodiments of the invention can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include topical administration, or coating of a device to be placed within the subject. In embodiments, administration is effected by injection or via a catheter.

Injectable drug delivery systems may be employed in the methods described herein include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Shienoes, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

The dosage of a compound of the invention administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds of the invention may comprise a compound alone, or mixtures of a compound with additional compounds used to treat COPD. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

A compound of the invention can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

A compound of the invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

A compound of the invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, a compound may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain a compound of the invention and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, a compound may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

A compound may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions thereof of the invention can be coated onto stents for temporary or permanent implantation into the cardiovascular system of a subject.

Aspects of the present invention are also described in: Hirabayashi et al., (2013) Transformed Drosophila Cells Evade Diet-Mediated Insulin Resistance through Wingless Signaling, Cell (In Press), dx.doi.org/10.1016/j.cell.2013.06.030, the entire contents of which are incorporated herein by reference.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLE 1

### Treatment of COPD with LXR Agonists

Innovation

A recent study by the inventors (Goldklang et al., 2007) identified that ApoE KO mice fed a Western-type diet develop severe systemic hypercholesterolemia suggesting that abnormal cholesterol efflux can then induce pulmonary inflammation through a TLR4/inflammatory/MMP cascade, ultimately resulting in the development of emphysema (Geraghty et al., 2001; Tall et al., 2002). ApoE promotes macrophage cholesterol efflux via the ABCA1 and ABCG1 cell surface transporters, initiating the formation of HDL particles (Global Initiative for chronic obstructive Lung disease, 2007; Yoshida et al., 2007; Tall et al. , 2007; Ranalletta et al., 20061 Tall et al., 20081 Wang et al., 2004; Yvan-Charvet et al., 2010; Yvan-Charvet et al., 2010a). Deficiency of ABCA1 and ABCG1 results in a significant decrease in macrophage cholesterol efflux, demonstrating that these two receptors play a major role in cholesterol efflux from foam cells to plasma lipoproteins (Yvan-Charvet et al., 2007). ABCA1 and ABCG1 KO mice manifesting abnormal cholesterol efflux exhibit pulmonary abnormalities (Baldan et al., 2007; Bates et al., 2005). Macrophages from these mice exhibit increased expression of inflammatory and oxidative stress genes via TLR signaling, suggesting a link between alterations in cholesterol efflux and lung inflammation through TLR signaling (Yvan-Charvet et al., 2010; Yvan-Charvet et al., 2010a; Yvan-Charvet et al., 2008). In fact, the excessive presence of foamy macrophages coupled with the upregulation of MMP-9 and MMP-12 was demonstrated in cigarette-smoke induced emphysema in mice (Hirama et al., 2007). Additionally, ABC transporters were found to negatively correlate with a TLR-dependent inflammatory response (Yvan-Charvet et al., 2008) and are major transporters of S1P (the by-product of ceramide), suggesting a potential role for these transporters and linking inflammation and lipid homeostasis in the lung upon cigarette smoke exposure. These events then in combination can play a role in the increased risk of emphysema seen in smokers and potentially provide novel insight into a new therapeutic target to consider for treatment of the disease.

Thus, the work described below fills a critical gap in the existing literature and provides insight into how cigarette smoke induced impaired lung lipid homeostasis affects emphysema development. Aspects of the present invention provide novel pharmacotherapies relating to these biological processes.

Approach

The studies presented herein improve the understanding of the role of cholesterol homeostasis in the lung and explain the link between cigarette smoke induced abnormal cholesterol efflux, lipid homeostasis and inflammation driven emphysema pathogenesis. The data herein suggests that ABC transporters link macrophage cholesterol efflux with cigarette smoke induced inflammation and MMP production. The role of ABC transporters in emphysema pathogenesis is determined, and their effect on inflammation, lung destruction and sphingolipid homeostasis, all of which are considered to be crucial in emphysema development, is evaluated. Macrophages and epithelial cell specific single or double ABCA1/G1 (mice were kindly provided by Dr. Alan Tall, Department of Medicine, Columbia University) deletion strategy or activation of ABCA1/G1 (LXR agonists, miR-33) are utilized to achieve the goals described below in the aims.

Specific Aim I: Testing the Hypothesis that Cigarette Smoke Regulates ABCA1 Transporters Which then Modulates Lung Inflammatory and Tissue Destructive Pathways

The initial goal is to determine the role of ABC transporters as a part of lipid homeostasis in cigarette smoke induced activation of inflammatory and tissue destructive signaling pathways. Their role in emphysema development and progression is ultimately defined, and identifies novel therapeutic targets in the disease. In a previously published study, the inventors presented a link between impaired cholesterol transport and emphysema development (Goldklang et al., 2012). Reverse cholesterol transport, or efflux, is controlled by two ATP-binding cassette transporters, ABCA1 and ABCG1 (Wang et al., 2004; Yvan-Charvet et al., 2007; Adorni et al., 2007; Beaven et al., 2006). Therefore, the effect of cigarette smoke on transporter expression is investigated.

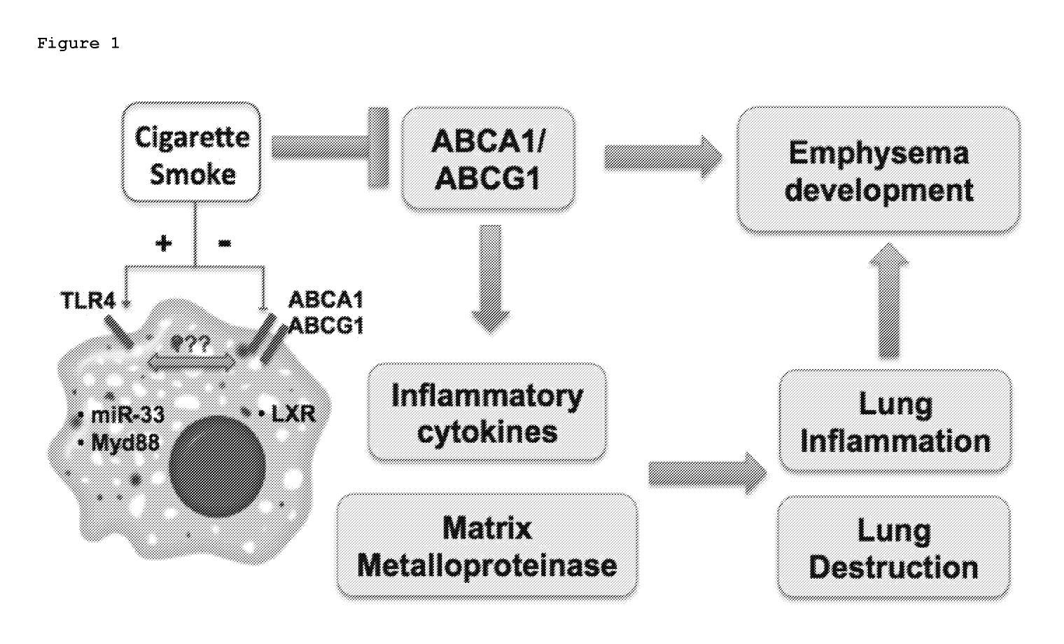

In Aim I the goal is to determine the ABCA1/G1-driven impairment of the cholesterol efflux mechanism in macrophages during cigarette smoke exposure. A mechanistic link is established between the cigarette smoke-induced abnormal cholesterol efflux and the TLR/Myd88 pro-inflammatory response coupled with increased MMP secretion in vitro in macrophages and ultimately in vivo in the cigarette smoke emphysema model (FIG. 1.).

Initial Studies:

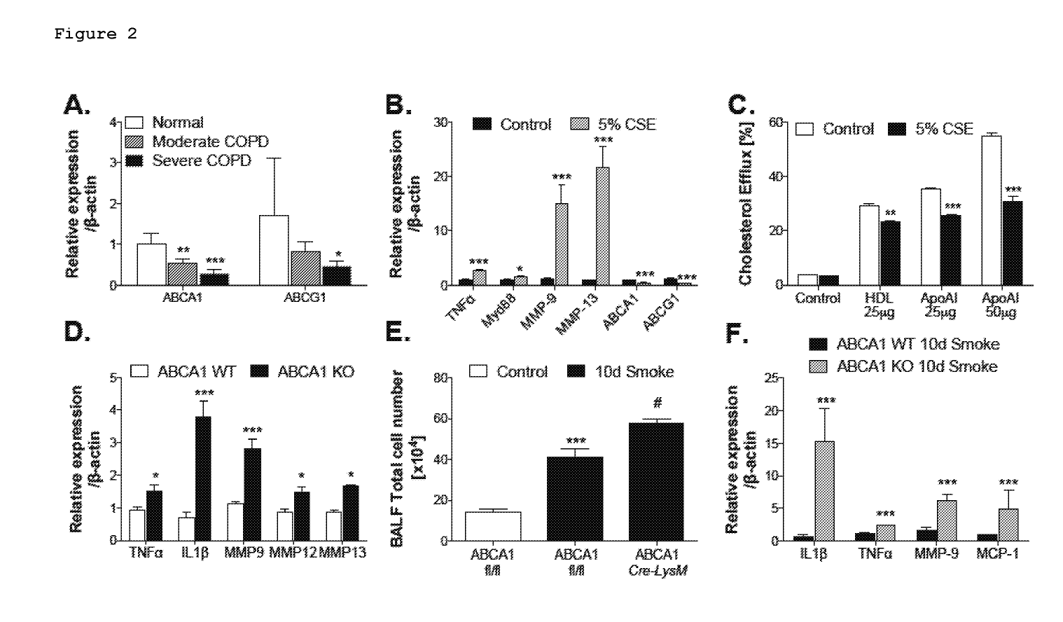

In both murine smoke exposure models and lungs of patients with COPD, the TLR/Myd88 pathway is activated and MMP expression increased. The initial results described herein show that there is a substantial decrease in ABC transporter expression in the lungs of COPD patients (FIG. 2 A), In addition, treatment of macrophages with cigarette smoke extract (CSE) exhibited a significant decrease in ABC transporter expression as well as increased expression of MMPs and Myd88 (FIG. 2 B.). Downregulation of ABC transporters was confirmed by demonstrating decreased cholesterol efflux (Yvan-Charvet et al., 2007) to ApoAI and HDL in the cigarette smoke exposed macrophages (FIG. 2C.).

Interestingly, macrophages deficient in ABCA1 (ABCA1 Cre-LysM) exhibit increased expression of inflammatory genes such as TNF, IL-1 and MMPs even without CSE treatment (FIG. 2D.), Furthermore, acute cigarette smoke exposure (10 days) of mice with a macrophage specific deletion of ABCA1 resulted in considerable amplification of the inflammatory response in the lung presenting an increase in total inflammatory cells in the BAL fluid (FIG. 2E.). Expression of pro-inflammatory cytokines and MMPs were measured in the BAL and revealed upregulation of IL-1, TNF, MCP-1 and MMP-9 (FIG. 2F.) in the mice with a macrophage specific deletion of ABCA1 which overall suggests that cholesterol efflux mechanisms regulated by ABC transporters are closely linked to inflammation and MMP induction in the setting of cigarette smoke exposure.

Aim 1a: Defining the Cigarette Smoke Dependent Regulation of ABC Transporters in Alveolar Macrophages.

As described above, in vitro cigarette smoke inhibition of ABCA1 and ABCG1 is coupled with a decrease in cholesterol efflux in macrophages and an increase in cigarette smoke induced inflammation (FIG. 1). Therefore, the first part of this aim examines the mechanisms involved in the cigarette smoke (CSE) induced inhibition of ABC transporters and subsequently the role of transporter deficiency in inflammatory pathways and MMP activation in alveolar macrophages. Three of the major known pathways that regulate cholesterol efflux and ABC transporters expression are focused on: (1) liver X receptor (LXR); (2) micro RNA-33; and (3) TLR4/Myd88 signaling pathways to determine which of these contributes to ABC transporter down-regulation secondary to cigarette smoke exposure.

Liver X Receptor. Briefly, LXR nuclear hormone receptors regulate cholesterol homeostasis in response to cholesterol excess by inducing the expression of genes involved in cholesterol efflux, including ABCA1 and ABCG1 (Beaven et al., 2006; Larrede et al., 2009). The expression profile of LXRα and p and their target genes ABCA1 and ABCG1 are examined by Real Time PCR (ABI) and Western Blotting (Biorad) after the stimulation of macrophages with 5% CSE. Protein levels of LXR are measured in the cytosolic as well as nuclear fractions. The activity of LXR is measured by an LXR Cignal Reporter Assay (Sabiosciences). In addition, whether the regulation of ABCA1 is LXR dependent is assessed utilizing a luciferase reporter assay with the wt-ABCA1 promoter construct and an ABCA1 promoter construct containing a mutation in the LXR responsive direct-repeat-4 (DR4) promoter element (both constructs have kindly been provided by Dr. Alan Tall, Chief of Molecular Medicine, Columbia University).

microRNA-33. Recent studies have shown that miR-33 inhibits the translation of ABCA1 and reduces reverse cellular cholesterol transport in vivo (Rayner et al., 2010). Involvement of miR-33 in cigarette smoke regulation of ABC transporters is examined with the use of anti miR-33 siRNA custom designed by Ambion.

TLR4/Myd88 pathway. Recent published studies of the inventors demonstrate that the TLR4 pathway is an important upstream signaling molecule of the inflammatory and protease production that occurs in the pathogenesis of COPD (Karimi et al., 2006; Geraghty et al., 2011). The inventors suggested that the TLR signaling pathway is able to couple reverse cholesterol transport to inflammation and regulate ABC transporter expression (Castrillo et al., 2003). Additionally, the inventor's new data suggest that the TLR4 signaling pathway is a probable candidate to serve as the initiator of the upstream signaling events linking the inflammatory and cholesterol pathways (Yvan-Charvet et al., 2008).

Therefore, the interaction between the TLR4/Myd88 signaling pathway, cigarette smoke and the regulation of ABC transporters is examined extensively using macrophages isolated from TLR4 and Myd88 KO mice as well as with the use of TLR4 neutralizing antibody and IRAK inhibitor. The effect of the above pathways on ABC transporters expression after 5% CSE treatment in macrophages is tested using RT-PCR analysis utilizing Taqman probes for ABCA1 and ABCG1 as well as western blot and immunocytochemistry analysis using ABCA1 and ABCG1 antibodies (Novus Biologics). In addition to the below described experiments, the effect of LXR (use of LXR agonist), micro RNA-33 (use of anti-mir-33) and TLR4/Myd88 (use of TLR4 neutralizing antibody, IRAK inhibitor, Myd88 KO macrophages) on the cholesterol efflux and efferocytosis ability of macrophages is analyzed to determine whether reversing the pathways affected by cigarette smoke exposure restores cholesterol efflux and improves phagocytic properties of alveolar macrophages.

Aim 1b: Determining the Role of ABC Transporters Deficiency on Cigarette Smoke Dependent Inflammatory Pathways, MMP Induction and Efferocytosis in Macrophages.

Mice lacking ABCA1 and ABCG1 accumulate inflammatory macrophage foam cells in various tissues such as in the lung and liver (Yvan-Charvet et al., 2007; Yvan-Charvet et al., 2008). Several investigators have focused on the lung because mice lacking ABCA1, with low plasma HDL levels, developed pulmonary lipidosis and progressive disease with chronic inflammation (Baldan et al., 2008; Bates et al., 2005). Without wishing to be bound by any scientific theory, inventors hypothesize that the mechanism by which these transporters contribute to cigarette smoke induced lung diseases is similar to what has been described for atherosclerosis, including enhanced TLR-mediated macrophage inflammation and tissue destructive MMP secretion (FIG. 1.).

Previously, investigators demonstrated that macrophages deficient in ABC transporters are more sensitized to TLR signaling localized to lipid rafts. Furthermore, LPS, which decreases ABCA1/G1, activates macrophages with a transient increase in TLR4 trafficking to lipid rafts (Zhu et al., 2008) along with its cognate adaptor proteins such as Myd88 and subsequent secretion of inflammatory cytokines and chemokines (Zhu et al., 2008). These studies suggest a link between ABC-mediated cellular lipid efflux, membrane lipid raft homeostasis, efferocytosis and overall activation of macrophages. Therefore, examine the effect of cigarette smoke on this process is examined.

Whether cigarette smoke induced expression of TLR4/Myd88, inflammatory cytokines and MMPs are regulated in an ABC transporter dependent fashion is determined. To achieve these goals macrophages isolated from ABCA1 and G1 fl/fl mice as well as ABCA1/G1 fl/fl (as WT) and macrophage specific ABCA1, G1 single KO or double KO mice (Cre-LysM-ABCA1) are utilized. How ABCA1 deficiency in macrophages affects TLR4/Myd88/MMP pathways and inflammatory cytokine secretion with or without the presence of CSE is also determined.

TLR4/Myd88 pathway. The molecular details regarding how ABCA1 expression affects macrophage TLR-dependent inflammatory response are poorly understood. To define the cigarette smoke ABCA1-dependent regulation of TLR4/Myd88 signaling, ABCA1 WT and KO macrophages are exposed to cigarette smoke. Initially, the expression profile of TLR4 and Myd88 are analyzed by RT-PCR and western blotting in WT and KO macrophages with or without CSE. In addition, the content of TLR4 in the lipid rafts of ABCA1 deficient macrophages in response to CSE is analyzed. To induce translocation of TLR4 to lipid rafts, BMDMs from both WT and ABCA1 KO mice are incubated in 1% NutSP-RPMI-1640 media overnight and then treated with ±100 ng/ml LPS from Salmonella typhimurium (Sigma-Aldrich) as a known positive control or 5% CSE for 24 h, followed by the preparation of lipid rafts and nonrafts as previously described (Zhu et al., 2010). Macrophage lipid raft TLR content is calculated as a percentage of total membrane TLR (i.e., lipid rafts +non-raft fractions) as previously described (Zhu et al., 2010). Furthermore, the surface expression of TLR4 in response to cigarette smoke is determined in ABCA1 deficient and WT macrophages by FACS (Zhou et al., 2011) and by basic immunocytofluorescence. To test the dependency of the above pathway on Myd88, a threefold blocking strategy is utilized with the use of: (1) siRNA (Santa Cruz, CA); (2) Myd88 blocking peptide (Invivogen); and (3) macrophages isolated from mice macrophage specific triple deficient in ABCA1/G1 and Myd88 (Cre-LysM-ABCA1/G1/Myd88).

MMP analysis. To test the hypothesis that cigarette smoke-induced MMP secretion is regulated by ABCA1, ABCA1 deficient and WT macrophages are treated with or without CSE and LXR agonist (a potent ABCA1 inducer). In initial studies, MMP-9 expression was observed to be upregulated in ABCA1 KO macrophages and its CSE-induced activity in macrophages can be blocked by LXR agonism in WT but not ABCA1 deficient macrophages suggesting ABCA1-dependence (FIG. 2). In this part of the study, experiments are designed with the intent to determine the mRNA and protein expression of MMP-9, -12, -13 (significant emphysema contributors) in macrophages (ABCA1 WT vs KO) exposed to cigarette smoke using a methodology published by the inventors. Active protease expression is demonstrated by zymography.

At the same time levels of TIMPs (1, 2, and 3) are examined to determine if a protease imbalance exists.

Cytokines analysis. Cytokines that are upregulated by cigarette smoke and shown to be important in emphysema development such as TNF-alpha, Interferon Gamma and Interleukins (-1, -6, -8, -10, -13) are analyzed in CSE-exposed macrophages (ABCA1 WT vs KO) with or without LXR agonist by RTPCR (mRNA) and ELISA's.

In addition to above described experiment the role of ABC transporters on cigarette smoke impaired-efferocytosis capability of macrophages is determined as impaired phagocytosis of apoptotic cells has been recently implicated to be great contributor to in COPD state (Petrusca et al., 2010; Dehle et al., 2013).

Aim 1c: Determining the Role of Macrophage Specific ABC Transporters Deficiency on Cigarette Smoke Induced Emphysema.

In this sub-aim, the role of the ABC transporters deficiency in macrophages and epithelial cells in vivo on emphysema development in a chronic cigarette smoke exposure mouse model (8 months) is determined. Whether the administration of LXR agonist by its dual action of restoring ABCA1-dependent cholesterol efflux and blocking the TLR/MMP driven pro-inflammatory/tissue destructive signaling in the lung is a useful therapeutic approach to treat cigarette smoke induced emphysema is determined.

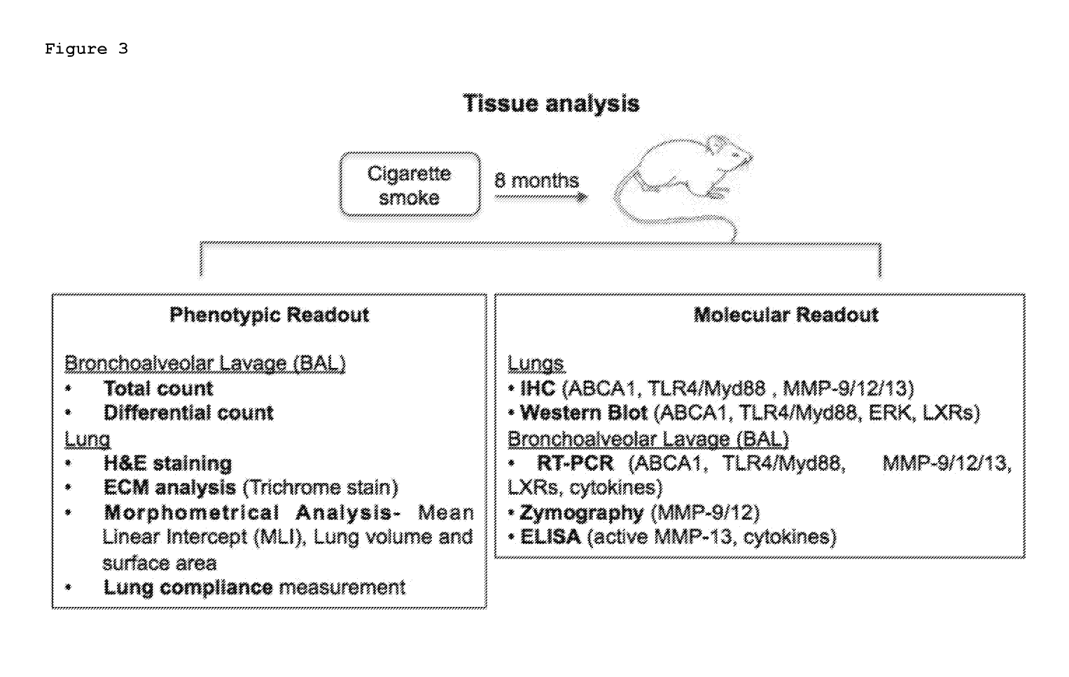

Eight week-old ABCA1, ABCG1 single and ABCA1/G1 double macrophage (Cre-LysM) and lung epithelial cell-deficient mice (Cre-SP-C) and littermate controls (ABCA1, ABCG1, ABCA1/G1 fl/fl) on a C57BL6/J background are used in the study and exposed to room air (n=15 per group) and cigarette smoke (n=15 per group) which in total gives 270 mice. After 8 months of exposure the remaining mice are sacrificed to evaluate for structural lung changes consistent with emphysema as previously published (D'Armiento et al., 1992) (FIG. 3.).

Methods: All methods performed such as the preparation of cigarette smoke extract (CSE), RT-PCR, Western Blotting, transient transfection are within the inventors' expertise and in its published studies (Foronjy et al., 2008; Foronjy et al., 2005; Foronjy et al., 2006; Foronjy et al., 2003; Geraghty et al., 2011; Golovatch et al., 2009). General procedures are described briefly below:

Alveolar macrophages and epithelial cell culture. In these experiments, freshly isolated murine alveolar macrophages (AM) and lung epithelial cells (EC) are used. Mouse AMs are obtained from the following mice in a pure C57BL/6 background (Jackson Laboratories, ME): macrophage and epithelial specific ABCA1, ABCG1 single KO (SKO), ABCA1/G1 double KO (DKO) and ABCA1/G1/Myd88 triple KO (TKO). They are compared to their WT control fl/fl littermates such as ABCA1, ABCG1 single fl/fl, double fl/fl and ABCA1/G1/Myd88 triple fl/fl. Briefly, trachea are cannulated with catheter to perform BAL. 8 washes with 1 ml PBS-EDTA are performed followed by centrifuge of lavage at 450 g 10 min. Cells are re-suspended in warm RPMI in a concentration of 2×10⁶ cells/ml. Cells are placed into incubator for 45 minutes to allow macrophages to adhere and media is changed adequately to stimulations. This protocol should typically yield 0.3×10⁶ cells per mouse with 98% AM purity and 96% viability. Epithelial cells from mouse lung are isolated by CellBiologics, IL to provide equal, established and guaranteed conditions for the experiments described in this study.

After nine months of cigarette and room air exposure mice are sacrificed and analyzed according to standard methods previously published (D'Armiento et al., 1992) by the inventors and illustrated in FIG. 3. General methods are briefly described below:

Cigarette smoke exposure and emphysema analysis. Mice are exposed to chronic smoke exposure in a specially designed chamber (Teague Enterprise, Calif.). Eight week-old mice are smoke-exposed for 5 hours a day, 5 days a week, for 9 months. The total particulate matter (TPM) within the smoking chamber is regulated such that mice receive a TPM of 80-120 mg/m³. TPM is determined by a gravimetric analysis of filter samples taken during the exposure period. Control mice are exposed to room air. After 9 months of exposure to cigarette smoke, the lungs of mice are sectioned and stained with hematoxylin and eosin (H&E) to perform morphometric analysis. Standard measurements are made by determining the mean linear intercept (Thurlbeck method) (Im et al., 2011).

Determination of lung compliance. To determine the pulmonary compliance of the lung, a closed chest model is used. Respiratory mechanics are assessed using a flexiVent (SCIREQ) system. Pentobarbital is used for sedation, and the mice undergo neck dissection and tracheostomy. Following this, the mouse is paralyzed with succinylcholine and then a full assessment of pulmonary mechanics in triplicate utilizing the flexiVent system is obtained. Following airway measurements, animals are sacrificed by CO2 inhalation and tissue analysis is performed as previously described.

Extracellular Matrix Analysis. Using methods previously published in papers from the inventors (Foronjy et al., 2003), extracellular matrix content and protease activity will be analyzed. The activity of collagenases and elastases and levels of collagens and elastins determined (Woessner et al., 1961). To determine collagen type III/type I ratio, tissue sections for type III collagen are stained.

Analysis of MMP and cytokines profile. To measure MMP expression profiles, BAL cells and lung homogenates are analyzed by RT-PCR and Western Blotting (only lung). Activity is examined by gel zymographies that can detect MMP-2, -9, and -13 (gelatin and casein). In addition activity of MMP-12 and -13 is confirmed using Sensolyte 490 MMP-12 assay kit (Funakoshi) and active MMP-13 ELISA (R&D). Inflammatory cytokines profiles such as IL-1, -6, -8, -12, -13, TNF-alpha, IFN-gamma are tested by RT-PCR and ELISA kits (both BAL and lung).

Outcomes and Alternative Strategies.

After the completion of the above aim the mechanism of regulation of ABC transporters by cigarette smoke is defined in vitro. Furthermore, the consequences of impaired ABC-regulated cholesterol efflux in macrophages on cigarette smoke induced activation state of activation (phenotype of macrophages found in COPD patients) is demonstrated. Finally the role of ABC transporters deficiency in cigarette smoke emphysema development is determined. Through cigarette smoke exposure of macrophage specific ABC transporters KO mice, a more potent inflammatory response is observed in the lung due to the increased inflammatory cytokines and MMP expression, allowing for maximum macrophage infiltration to the lung and progression of emphysema formation.

Number of Mice and Statistical Analysis.

For in vitro study all experiments are assayed in triplicates, and each experiment is repeated at least three times. Statistical analysis of the data obtained in this part of the proposal is performed using the unpaired two-tailed Student's T-test when 2 groups are being compared. In case of three or more groups being compared one-way ANOVA followed by the Bonferroni post hoc test is performed using GraphPad Prism software. For in vivo studies, 15 mice are analyzed in each group. An average and standard deviation for each type of measurement is calculated. A 10-15% change in mean linear intercept due to smoke exposure is identified. In order to have an 80% power to detect a 20% difference in morphometry between smoke-exposed mice and control animals, a total of 15 animals will is required in each subgroup (Alpha=.05, 1-Beta=2)

Specific Aim II: Testing the Hypothesis that Loss of ABC Transporters Leads to Increased Lung Destruction in Cigarette Smoke Exposed Mice.

The decrease in ABCA1 and ABCG1 in the lung of COPD patients identified in the initial studies described herein suggests that agents that restore expression of these transporters could be a potential therapeutic approach of ABC transporter-dependent mechanisms in emphysema development. Targeting cholesterol efflux mechanisms, which directly or indirectly reestablish ABC transporters expression and function, proved to be very effective in atherosclerosis regression (Tall et al., 2007; Yvan-Charvet et al., 2010; Yvan-Charvet et al., 2007; Libby et al., 2002; Plump et al., 1994) including techniques such as LXR agonism (Im et al., 2011, Joseph et al., 2002, Levin et al., 2005; Wang et al., 2006) as well as therapy targeting miR-33 to increase ABC transporter levels (Rayner et al., 2010; Rayner et al., 2011). Both LXR and miR-33 directly control transcriptional regulation ABCA1 and ABCG1 (Tall et al., 2008; Larrede et al., 2009; Rayner et al., 2010; Rayner et al., 2011; Levin et al., 2005) and establish improvement in atherosclerosis parameters such as lesion size, regression of inflammation and collagen deposition (Joseph et al., 2002; Rayner et al., 2011). In this aim we in vivo studies further examining the effect of ABC transporter modulation by LXR agonist and anti miR-33 treatment on inflammatory signaling pathways, MMP induction and ultimately on emphysema development are performed.

Initial Studies

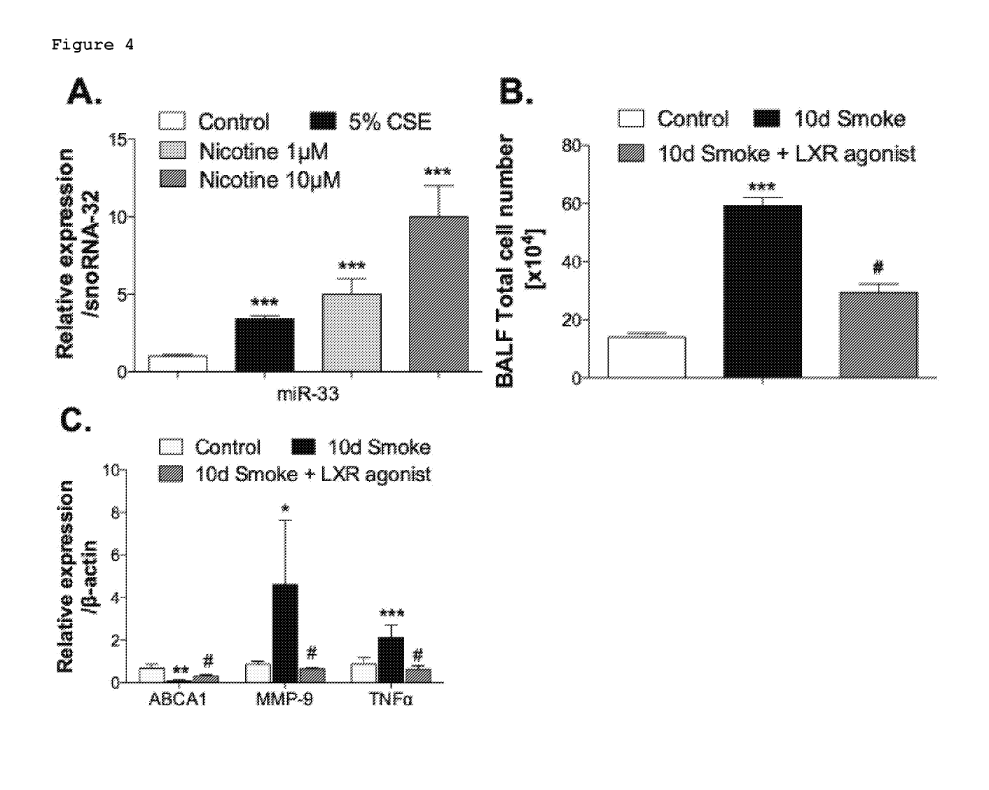

ABCA1 and ABCG1 are transcriptional targets of LXR activation (58). While increased expression of miR-33 was shown to repress the expression of these transporters (Rayner et al., 2010), the cholesterol efflux ability of macrophages was affected (Tall et al., 2008; Larrede et al., 2009; Rayner et al., 2010). This study shows that treatment of macrophages with CSE and nicotine resulted in increased miR-33 expression (FIG. 4A.). This correlated with decreased ABC transporter levels and macrophage cholesterol efflux ability (FIG. 2.), LXR treatment of macrophages re-established reduced ABC transporter expression from cigarette smoke (data not shown). In addition LXR agonist treatment (25 mg/kg, IP injection) of mice acutely exposed to cigarette smoke for 10 days significantly blocked pulmonary inflammation (FIG. 4B.), correlating with re-expression of ABCA1 and inhibition of MMP-9 and TNFα in BAL isolated alveolar macrophages (FIG. 4C.).

The data herein clearly show that modulation of ABC transporters by LXR agonism or targeting miR-33 is a novel therapeutic approach to block inflammatory signals and MMP induction which can block emphysema progression.

Approach/Methods

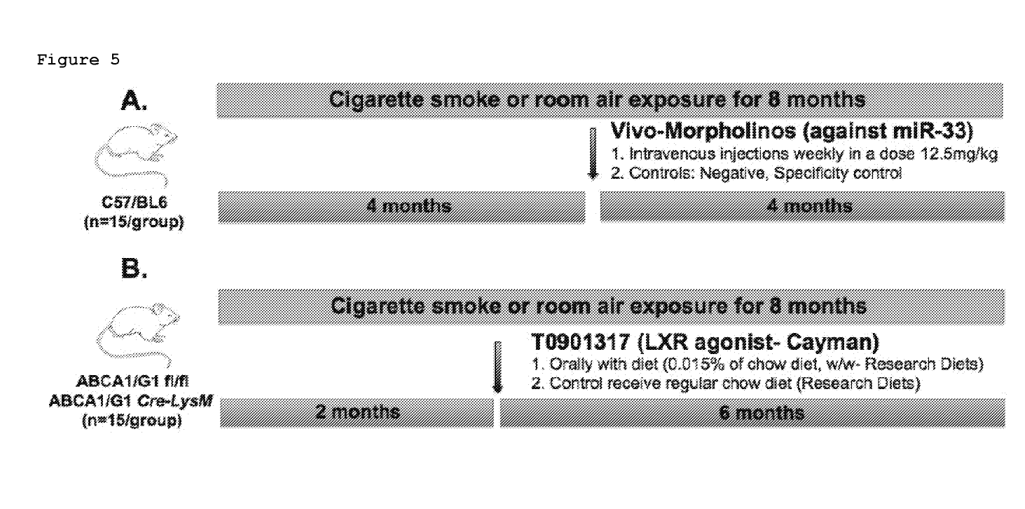

In this aim ABC transporters expression is modulated in the chronic murine cigarette smoke exposure model (FIG. 5.) using both miR-33 antagonism (FIG. 5A.) and LXR agonism (FIG. 5B.). After 8 months of exposure the mice are sacrificed to evaluate for structural lung changes and lung mechanics consistent with emphysema (FIG. 3.) as previously published (D'Armiento et al., 1992) and according to approaches briefly described below:

Anti-miR-33 treatment. miR-33 is antagonized with Anti-miR-33 and weekly injections are performed according to procedure described previously with Vivo-Morpholinos, 12.5 mg/kg for 4 months (FIG. 5.) by weekly tail vein injection (Morcos et al., 2008; Moulton et al., 2009). After 4 months of cigarette smoke exposure mice are divided into three groups and the sequence of miRNA injected is as follows: negative control (originally targeted to a human intronic mutation in beta-globin) (n=15), specificity control Vivo-Morpholino oligomer (TTA TCG CCA TGT CCA ATG AGG CT) (SEQ ID NO:1) (n=15), or miR-33 targeted Vivo-Morpholino oligomer (TGC AAT GCA ACT ACA ATG CAC) (SEQ ID NO:2) oligonucleotide (Morcos et al., 2008; Moulton et al., 2009) (n=15) (Total=60 mice). Injections are performed weekly and continue for last 4 months of cigarette smoke exposure. Mice after 8 months of cigarette smoke exposure are sacrificed and analyzed as described above (FIG. 5.).

LXR agonist treatment. Eight week-old macrophage (Cre-LysM) or epithelial (Cre-SP-C) ABC transporters deficient mice and littermate controls (ABCA1/G1 fl/fl) on a C57BL6/J background are used in the study and exposed to four different conditions: room air (n=15), room air with additional treatment with LXR agonist (n=15), cigarette smoke (n=15), cigarette smoke with additional treatment with LXR agonist (n=15) (Total=180 mice). Treatment starts after 3 months of cigarette smoke exposure, when the inflammatory response is already established, and continued for up to 8 month of cigarette smoke exposure. The drug is delivered in the diet. The LXR agonist concentration is calculated based on the average mouse weight (30 g) and on average mouse food consumption (6 g/day). Experimental food is prepared with a 0.015% LXR agonist (T0901317-Cayman) in food (w/w) concentration corresponding to 30 mg/kg body weight. Both the control group and the custom drug diet are prepared by Research diets.

Outcomes and Alternative Strategies.

The inventorshave extensive experience with the murine cigarette smoke exposure model, and as such, these experiments are completed successfully. Without wishing to be bound by any scientific theoy, the loss of macrophage or epithelial cell ABC transporters significantly enhances the development of cigarette smoke induced emphysema. In contrast, the treatment of cigarette smoke exposed control mice with the LXR agonist or anti-miR-33 is protective for inflammation and emphysema development. The treatment of macrophage and epithelial cell specific ABC transporter deficient mice also determines whether the protective effect of cholesterol efflux modulation is directly dependent on the loss of ABCA1 and ABCG1 in macrophages. Investigation of secondary causes and an evaluation of anti-inflammatory signals that can be modulated by LXR agonism or Anti-miR-33 treatment is performed. One of those mechanisms can be inflammatory response acceleration by ABC transporters-dependent potentiation of TLR4/Myd88 signaling pathway. To achieve that alternative plan. ABCA1/G1 CreaLysM mice are crossed with Myd88 fl/fl mice to establish triple Cre-LysM ABCA1/G1/Myd88 KO mice. The hypothetical increased susceptibility to emphysema of ABCA1/G1 Cre-LysM mice is abrogated by lack of TLR4/Myd88 signaling pathway. Studies are performed to evaluate the role of ABC transporters in epithelial and macrophages cell specific ABC transporters deficient mice in respect to miR-33, which is a very expensive experiment.

Statistical Analysis.

Data obtained in this part of the study is processed using the unpaired one-way ANOVA with Bonferroni post hoc test using GraphPad Prism software. To calculate sample size a parameter of mean linear intercept is used. To calculate the power the logarithm of measured values with the additive error model is analyzed. The mean values were indicated as logarithms. For variance mean of the relative variances is used. Having 15 animals per group is enables the detection of an effect of 0.25 (equal to a 16% difference) with a power of 95%.

Specific Aim III: Determining the Role of ABC Transporters in Sphingolipids Turnover in the Emphysematous Lung upon Cigarette Smoke Exposure.

Sphingolipid metabolites including ceramide, sphingosine, ceramide-1-phosphate (C1P) and sphingosine-1-phosphate (S1P) are not only components of the eukaryotic cell membrane, but also important bioactive signaling molecules, which regulate a diverse array of biological responses (Liu et al., 2012). Ceramides were reported to have a damaging effect on the lung and when exogenously administered led to emphysema (Petrache et al., 2005). Furthermore the by-product S1P reversed emphysema in a VEGFR blockade emphysema model in mice (Petrache et al., 2005). In addition, S1P is a blood borne, lysophospholipid mediator that exerts pleiotropic activities in a variety of cell types (Okamoto et al., 2011). S1P is released from activated platelets and presents in the plasma largely bound to plasma proteins and HDL (Okamoto et al., 2011), which appears to be the most prominent plasma carrier of S1P (Okamoto et al., 2011). In agreement with this finding, plasma S1P levels correlated with HDL (Liu et. al., 2012). This finding, coupled with the fact that HDL levels are significantly diminished in smokers, raised the inventors' interest towards ABC transporters in emphysema pathogenesis (Moffatt et al., 2004). ABC transporters are linked to sphingomyelin and ceramide signaling and transport (Sano et al., 2007; Kobayashi et al., 2006; Ghering et al., 2006) but more interestingly the presented data indicates that they actively participate in the cellular export of Sip (Sato et al., 2007; Fletcher et al., 2010). This aim takes advantage of the inventors' established collaboration with Dr. William Blaner of Columbia University and allows the inventors to define the lipid profile of the smoke exposed lung using a metabolomics approach. These studies are performed under ABC transporter deficiency conditions so as to delineate the role of transporters in the alterations of lung lipid secondary to cigarette exposure.

Initial Studies

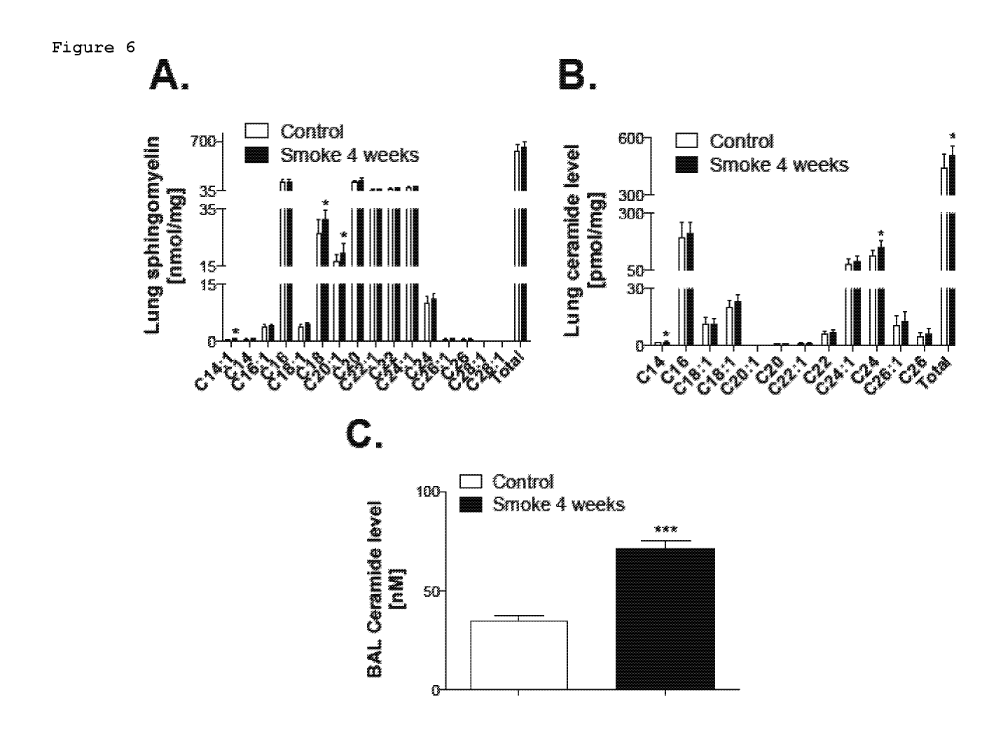

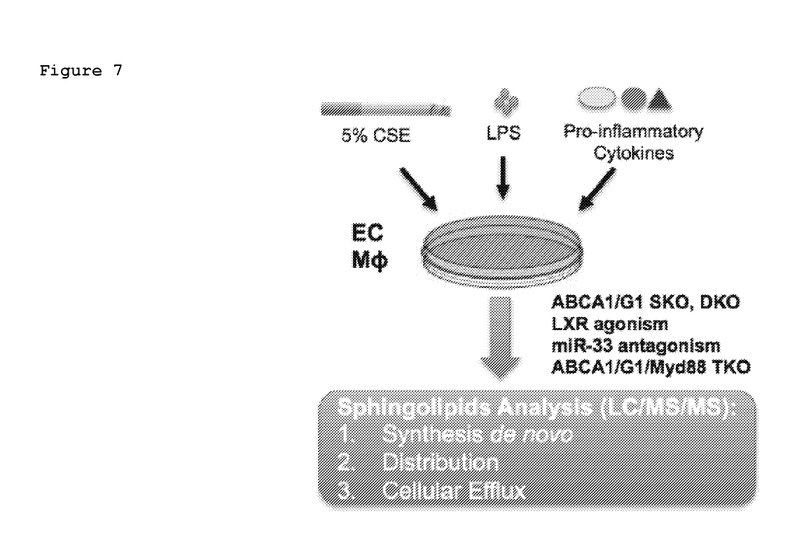

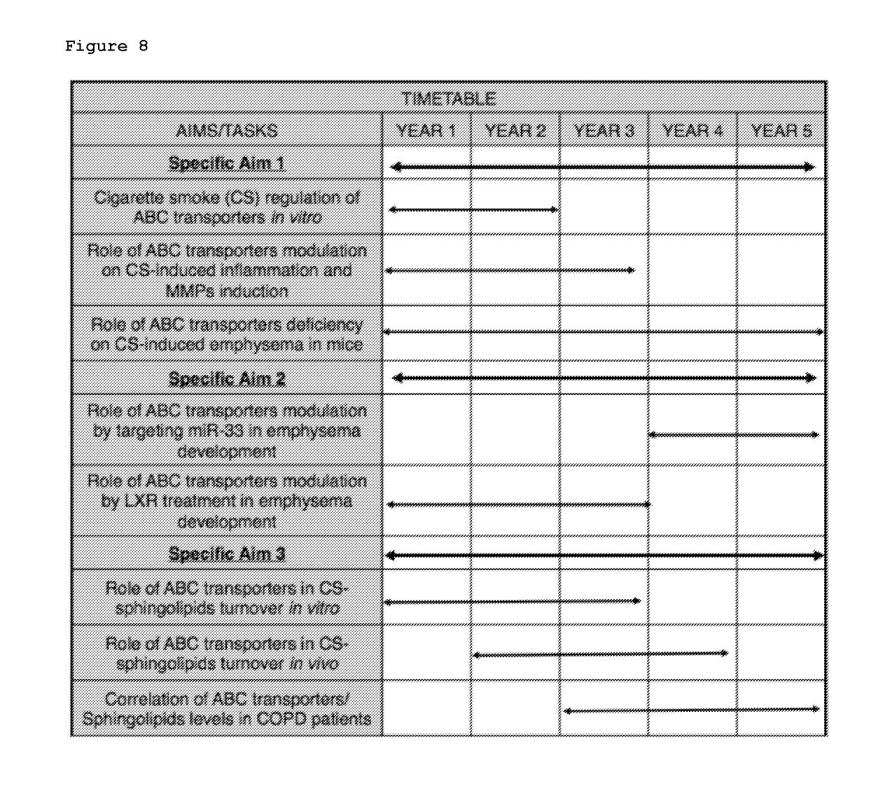

Due to the established collaboration with Dr. William Blaner the inventors are able to measure levels of ceramides and sphingomyelins in BAL, serum and lung tissue upon cigarette smoke exposure of mice. The initial results, similar to previously reported data (Petrache et al., 2005), demonstrate an increase in sphingomyelin (FIG. 6A.) and ceramide (FIG. 6B.) content in the lung upon cigarette smoke exposure. In addition, total ceramide levels were significantly increased in the BAL of smoke-exposed mice. Measurements were performed by LC/MS/MS in the lungs and BAL of mice exposed to cigarette smoke for 4 weeks as compared to their non-exposed controls (n=10).

Approach/Methods

Sphingolipids such as ceramide, while a relativly minor component of the lipid milieu in most tissues, may be among the most pathogenic lipids in general (Holland et al., 2008 Yang et al., 2011) with known detrimental effects on the lung architecture and emphysema pathogenesis (Petrache et al., 2005). Studies indicate an inverse relationship between sphingolipid de novo synthesis and cholesterol efflux. Inhibition of sphingolipid de-novo synthesis increases ABCA1 mediated cholesterol efflux independent of sphingemyelin, contrary to ABCG1 where it is sphingomyelin dependent (Worgall et al., 2011). The initial data shows that ABCA1 as well as ABCG1 are significantly downregulated in COPD patients (FIG. 2.). In addition to the role these transporters play in inflammation and MMP production, the diminished expression of ABC transporters in the lungs of smokers also prevents proper sphingolipid homeostasis as well as transport of protective S1P. Furthermore the results show that ABCA1 deficient macrophages exhibit increased expression of TNFα and IL-1β (FIG. 2.) which also contributes to increased ceramide production (27). This mechanism is evaluated in this study. The present aim seeks to determine the ability of ABC transporters to control sphingolipid levels at a cellular levels (alveolar macrophages and epithelial cells) as well as within the lung in a direct way or indirectly by affecting inflammatory pathways (TLR4/Myd88, TNFα, IL-1β). Ultimately the goal for this aim is determining the role of ABC transporters in regulating lung sphingolipid metabolite levels and transport such as ceramides and S1P to influence emphysema development and progression.

Sphingolipid analysis. Recently published work carried out in a mouse model of emphysema implicates sphingolipids, especially ceramides and the downstream pro-survivial metabolite sphingosine 1-phosphate (S1P), in the development and prevention of lung disease (Diab et al., 2010). This work provides strong evidence that S1P is important in ameliorating apoptotic processes important to emphysema development. It is also established in the literature that the ABCA1 transporter is needed for efficient S1P export from cells (Fletcher et al., 2010). Taken collectively, this information raises a question as to how cigarette smoke exposure affects sphingolipid levels in, and S1P efflux from, macrophages obtained from wild type and ABC transporter-deficient mice. Without wishing to be bound by any scientific theory, the inventors hypothesize that cigarette smoke markedly influences sphingolipid homeostasis in macrophages and that these are elevated in ABC transporter deficient macrophages that exhibit impaired S1P export. This hypothesis is tested in the present aim.

In initial studies, published targeted lipidomic approaches are employed involving the use of liquid chromatography tandem mass spectrometry (LC/MS/MS) methods developed in the Blaner laboratory (Clugston et al., 2011) to determine ceramide and sphingomyelin levels in serum and lungs obtained from mice exposed to cigarette smoke. Concentrations of sphingolipids in control and cigarette smoke treated macrophages and their culture media is surveyed to gain an understanding of how cigarette smoke and the presence or absence of ABC transporters may affect these levels. Specifically, these very sensitive LC/MS/MS protocols are employed to undertake quantitative analyses of sphingomyelin (C12:0 to C28:1 shingomyelin), sphingosine, sphingamine and their 1-phosphate metabolites, and ceramide (C12:0 to C28:1 ceramide) concentrations in pelleted washed macrophages and in parallel, their culture media.

These analytical methodologies are well established and require less than 100,000 cells or 100 μL culture media to allow for a complete quantitative analysis.

This exploratory survey is viewed as one that is needed to develop a more in depth understanding of potential relationships between cigarette smoke and the development of lung disease, rather than an endpoint per se. Sphingolipids are very potent regulators of cell signaling pathways, affecting both cell proliferation and apoptosis and the literature indicates an important role for sphingolipids in lung disease development. The studies are aimed at: 1. identifying the molecular basis for the observed treatment/genotype differences; 2. determining whether the differences observed are useful for therapeutic interventions; and 3. assessing whether the observed differences may serve as useful biomarkers for disease progression and/or responsiveness to therapies.

Aim 3a. Determining the Role of ABC Transporters in Sphingolipids Production in Response to Cigarette Smoke in Alveolar Macrophages and Epithelial Cells.

This part of the aim determines how the modulation of ABCA1 and ABCG1 in vitro in alveolar macrophages and epithelial cells affects sphingolipid turnover and de rove synthesis under CSE and proinflammatory conditions. For this purpose primary macrophages and pneumocytes freshly isolated from macrophage or epithelial cell specific ABCA1, ABCG1 single KO and ABCA/G1 double KO mice as compared to their proper fl/fl littermates are used. The cells are treated with various concentrations of CSE and proinflammatory cytokines that are known to be dysregulated in emphysema pathogenesis, such as IL-1β, IL-6, TNFα and TFNγ, to delineate the cell specific role of ABC transporters on sphingolipid production and their secretion (FIG. 7.). In addition, LPS, a known ceramide and sphingolipid modulator, is utilized to study the role of TLR/Myd88 signaling in cigarette smoke induced down regulation of ABC transporters and its effect on sphingolipid turnover.

Alveolar macrophages and epithelial cells are obtained as described in the methods section of Aim 1. Cells treated with the various approaches to modulate ABC transporters and dependent Myd88 (described above and on FIG. 7.) are treated with CSE, LPS and proinflammatory cytokines for subsequent sphingolipid (ceramides, sphingomyelin, S1P) analysis by LC/MS/MS. The role of ABC transporter deficiency and its modulation on their synthesis de novo (using labeled C13) is determined. Furthermore, their distribution in the cells is analyzed to determine which of the cells (AM or EC) are the main source of destructive sphingolipids in the lung (FIG. 7.).

In addition how ABC transporter deficiency and expression reestablishment affects cellular efflux (FIG. 7.) of sphingolipids that are identified to be deregulated after cigarette smoke in the initial study is analyzed (FIG. 6.). In addition, the ceramide metabolite S1P, shown to improve emphysema (Diab et al., 2010; Yasuo et al., 2013) and to be actively effluxed and processed by ABC transporters (Sato et al., 2007; Fletcher et al., 2010), is analyzed. Without wishing to be bound by any scientific theory, the inventors hypothesize that loss of ABC transporters in the lungs of COPD patients (FIG. 2.) is crucial to decreased S1P levels observed in cigarette smoke induced emphysema.

After the sphingolipids that are affected by cigarette smoke extract in alveolar macrophages and epithelial cells is delineated, the cells are then treated with the identified lipids and determine their effect on apoptosis (EC), efferocytosis (AM), MMP and proinflammatory cytokine production and induction of TLR4/Myd88 signaling. In addition, the modulation of ABC transporters by LXR agonism or miR-33 antagonism is shown to be an effective therapeutic approach in vitro against the detrimental effect of sphingolipids on AMs and ECs.

Aim 3b. Determining the Role of ABC Transporters in Sphingolipids Production in Emphysema Development.

This part of the aim determines how the deficiency of ABCA1 and ABCG1 in vivo in alveolar macrophages and epithelial cells affects sphingolipid turnover and their de novo synthesis in mice exposed to short-term (4 weeks) and chronic long-term (8 months) cigarette smoke. In addition, modulation of ABC transporters by miR-33 antagonism and LXR agonism is analyzed by LC/MS/MS to investigate sphingolipid turnover such as changes in ceramides, sphingomyelin and S1P in the lung and how this correlates with emphysema development in the mouse model.

Frozen samples from the lung and BAL of eight-week old mice exposed to cigarette smoke for 4 weeks in addition to the samples collected from mice described in Aims 1c., 2a., 2b. (exposed for 8 months) is analyzed for sphingolipid distribution using the LC/MS/MS. Since cigarette smoke led to significant changes in the sphingolipid profile of the lung and BAL (FIG. 6.), in this aim the role of ABC cholesterol transporters in the process of either sphingolipid de novo synthesis or degradation of the sphingolipids in the lung and how this change relates to emphysema development is evaluated. The mouse lungs are analyzed in the early stage of cigarette smoke exposure (4 weeks) as well as long term chronic exposure studies (8 months) utilizing the methods described in the previous aims with use of ABC transporters specific epithelial and macrophage knockout animals (Aim 1c.) as well modulation of this pathway using LXR agonism (Aim 2a.) and mir-33 (Aim 2 a.) and Myd88 (Aim 2 b.) antagonism. For the chronic studies samples obtained from the experiments described in the above aims are used.

Aim 3c. Testing the Hypothesis that Sphingolipids Levels Correlate with Loss of ABC Transporter Expression in Human Emphysema and Explore the Potential Use of This as a Biomarker.

This portion of the aim determines that the correlations observed in the cell culture and animal model translate into studies on human emphysema tissue compared to normal and lavage from patients compared to normal individuals. The serum is examined when the lung sphingolipid profile is defined, to target analysis of the plasma towards what is identified in the lung and lavage.

Over the last 15 years lung tissue samples have been collected from patients undergoing lung transplantation and lung volume reduction surgery. As described in the inventors' prior studies, these samples are de-identified and categorized based on histological severity of disease (Imai et al., 2005). Additionally, through participation in the FORTE study BAL samples were collected from patients with emphysema (Roth et al., 2006). These samples are also de-identified and stored at -80° C. The same samples utilized in the previous studies are examined to whether sphinoglipid molecules are useful biomarkers for disease severity and progression. These also correlate the levels of S1P and ceramides with loss of ABC transporters in the patient samples.

Outcomes and Alternative Plans

This aim identifies a clear relationship between cigarette smoke induced loss of ABCA1 and ABCG1 transporters and an altered sphingolipid profile in the lung observed in emphysema patients (FIG. 6. and (Diab et al., 2010)). The first part of the aim finds a significant increase in ceramide and sphingomyelin production in alveolar macrophages and epithelial cells. In addition S1P levels, which were observed to be reduced in the lung of cigarette exposed mice (Diab et al., 2010) are likely substantially reduced in the ABC transporter deficient mice in cigarette smoke exposed mouse lung and BAL. Modulation of ABC transporters by LXR agonism and miR-33 is observed to reverse the emphysema and correlate with the lung sphingolipid profile. Finally, by analyzing human lung and BAL samples for ABC transporter expression and the sphingolipid profile a correlation between loss of ABC transporters and a shift in sphingolipids is identified. These changes are correlated with emphysema progression and phenotype. The ABC transporters/sphingolipid ratio is associated with disease. This observation is pursued and these molecules are developed as therapeutics or biomarkers by linking to lung destruction. After the preliminary profile of the sphingolipids is obtained in lung disease and tissue, a larger future study is performed that correlates the sphingolipid profile with disease staging, severity and progression. Identification of a biomarker correlating with the extent of smoke exposure or lung damage is very valuable in the design of clinical trials.

### EXAMPLE 2

### Modulation of Cholesterol Efflux Through Treatment with an LXR Agonist in Cigarette Smoke Induced Emphysema in Mice

Methods and results: Studies were performed to investigate the link between cigarette smoke exposure, cholesterol efflux and inflammation in the lung. In the lungs of patients with COPD and BAL of murine smoke exposure models ABC transporter expression was down regulated and correlated with the level of inflammation and emphysema. Mouse macrophages were treated with cigarette smoke extract (CSE) and exhibited impaired cholesterol efflux and loss of ABC-transporter expression with up regulation of MMPs. By reestablishing ABC-dependent cholesterol efflux (by LXR agonist-T0901317-treatment), the cigarette smoke-induced pro-inflammatory cytokines and MMP expression and activity were blocked. To determine the effect. of ABC transporter expression reestablishment, treatment with an LXR agonist was performed on cigarette smoke induced lung injury in vivo (Acute—10 days, Chronic—5 months). In addition to the anti-inflammatory effect of the LXR agonist in the acute cigarette smoke exposure mouse model, administration (orally in diet, w/w 0.015%) of the LXR agonist exhibited an effect on chronic cigarette smoke exposure model for 5 months in AKR/J mice. Similar to the acute exposure studies, inflammation was blocked and led to a decrease in total inflammatory cell counts. In addition LXR agonist treatment decreased TNFα levels and induced ABCA1 expression in alveolar macrophages of BAL. The treatment with an LXR agonist also significantly improved pulmonary lung function with a decrease in lung compliance as compared to untreated smoke exposed mice.

Conclusions: The studies described above demonstrate an important association between cigarette smoke exposure and cholesterol mediated pathways. Importantly, modulation of these pathways by LXR agonism effectively blocked smoke induced inflammation and improved lung function in a chronic model of cigarette smoke exposure. These findings suggest that targeting cholesterol efflux through the use of an LXR agonist represents a novel therapeutic approach for the treatment of COPD.

LXR Agonists Attenuate Cigarette Smoke Induced Pulmonary Emphysema: a Role for ABC Transporters in Lung Disease

Introduction

Smoking related lung diseases, especially chronic obstructive pulmonary disease (COPD), is the third leading cause of death in the United States (Podowski et al. 2012; Mannino et al., 2007). Tobacco smoke is the key etiologic agent of COPD, which is characterized by inflammation, progressive airflow limitation and lung destruction (Global Initiative for Chronic Obstructive Lung Disease, 2011; Global Initiative for chronic obstructive Lung disease, 2007). Although COPD is defined clinically by airflow limitation, a mix of pathological findings are observed in the lung ranging from inflammation of the larger airways (termed chronic bronchitis), remodeling of the small airways, and parenchymal tissue destruction with airspace enlargement (defined as emphysema) (Global Initiative for Chronic Obstructive Lung Disease, 2011; Global Initiative chronic obstructive Lung disease, 2007). In addition to the changes seen in the lung patients with COPD exhibit systemic manifestations affecting skeletal muscles, bone and the cardiovascular system (Yoshida et al. 2007; Celli et al., 2006).

A protease/antiproteases imbalance was up to now a dominant paradigm explaining the pathogenesis of cigarette smoke-induced lung destruction where cigarette smoke exposure promotes repeated proteolytic injury to the extracellular matrix. The evolution of this paradigm is intimately linked to the recognition of the macrophage as a major effector cell; numbers within the alveolar walls correlate with emphysema severity (Finkelstein et al., 1995). Indeed, studies examining human alveolar macrophages and bronchoalveolar lavage fluid (BALF) have confirmed that a protease/antiprotease imbalance exists in cigarette-smoke-induced emphysema or COPD (Shapiro, 1999; Pons et al., 2005; Finlay et al. 1997; Finlay et al., 1997a). In particular, macrophages from the BALF of patients with emphysema exhibit increased MMP expression when compared with macrophages from normal subjects (Finlay et al. 1997; Woodruff et al., 2005). The D'Armiento Lab and others have documented the importance of MMPs in emphysema development (Woodruff et al., 2005; Foronjy et al., 2008; D'Armuiento et al., 1992; Hautamaki et al. 1997). Cigarette smoke is also associated with a significant increase in inflammatory responses including the secretion of cytokines such as TNFα (Yoshida et al., 2007; Thompson et al., 2012; Letuve et al., 2008), IL-1 (Churg et al., 2009; Couillin et al., 2009; Doz et al., 2008), IL-8 (Karimi et al., 2006), IL-13 (Zheng et al., 2000), IFNγ (Wang et al., 2000) and through induction of endogenous danger signals, sensed by pattern recognition receptors such as Toll-like receptors (TLR). Signaling through TLR4 triggers activation of macrophages leading to lung inflammation and a tissue destruction program via TLRs/MMP-1 (Geraghty et al., 2001) or IL-1/Myd88 (Couillin et al., 2009; Doz et al., 2008; Karimi et al., 2006) releasing MMPs from alveolar macrophages. The augmentation of lung inflammation, which goes along with a significant increase in protease activity, is a crucial factor in the resultant alveolar destruction that is characteristic of emphysema.

Multiple organ systems are affected by smoking, with smokers displaying higher susceptibility and increased severity of cardiovascular disease (Barnoya et al., 2005; Freund et al., 1993; Howard et al., 1998; Milei et al., 1998). A common feature of both emphysema and atherosclerosis is inflammation originating from the infiltration of macrophages and lymphocytes into the airway or vessel wall, respectively (Finkelstein et al., 1995; Finkelstein et al., 1995; Hansson et al. 2005). Of note, smokers with airflow limitation have more prominent atherosclerosis than smokers with normal lung function, suggesting a link between atherosclerosis and obstructive lung disease (Iwamoto et al. 2009). Atherosclerotic lesions exhibit increased numbers of lipid-laden macrophages (Hansson et al. 2005) with the accumulation of foamy alveolar macrophages observed in smoke-exposed mice (Hirama et al., 2007). Interestingly, passive smoking is known to influence plasma lipid concentrations (de Padua Mansur et al., 1997; Moskowitz et al. 1990), most significantly decreasing HDL levels (Moffatt et al., 2004). Cigarette smoke is also associated with increased levels of oxidized low-density lipoprotein (LDL) cholesterol, which damages the vessel endothelium (Stokes, 1990). However, despite the damaging effects of these lipids in the vascular wall, the consequences of systemic lipid changes have not been fully examined within the lung tissue. Lowering LDL cholesterol has little or no effect on pulmonary function; on the other hand, HDL regulates the immune system (Cirillo et al., 2002) and could have a protective effect on the lung during smoke exposure. HDL binds to bacterial toxins and diminishes inflammation; therefore, in smoke exposure decreased HDL could contribute to more inflammation within the lung (Cirillo et al., 2002). The major anti-atherogenic property of HDL is due to its ability to stimulate the release of cholesterol from activated cholesterol filled macrophages (cholesterol efflux), ultimately diminishing the inflammatory response (Yvan-Charvet et al., 2010). Two ATP-binding cassette transporters, ABCA1 and ABCG1, control this process of reverse cholesterol transport, or efflux (Adomi et al., 2007; Out et al., 2008; Wang at al., 2004; Yvan-Charvet ea al., 2007).

The D'Armiento laboratory observed that ApoE KO mice fed a Western-type diet develop severe systemic hypercholesterolemia accompanied by abnormal cholesterol efflux (Goldklang et al., 2012), inducing pulmonary inflammation through a TLR4/inflammatory/MMP cascade, all of which ultimately resulted in emphysema formation in ApoE KO mice (Geraghty et al., 2011; Tall et al, 2008). ApoE promotes macrophage cholesterol efflux via the ABCA1 and ABCG1 cell surface transporters, initiating the formation of HDL particles (Global Initiative for chronic obstructive Lung disease, 2007; Yoshida et al., 2007; Yvan-Charvet et al., 2010; Wang et al., 2004; Tall et al., 2002; Ranalletta et al., 2006; Tall et al., 2008; Yvan-Charvet et al., 2010a). Deficiency of ABCA1 and ABCG1 results in a significant decrease in macrophage cholesterol efflux (Yvan-Charvet et al., 2007) and the accumulation of lipids in macrophages induces an inflammatory response characterized by the secretion of cytokines and proteases (Libby et al., 2002). The present study was undertaken to examine the direct effect of cigarette smoke on the efflux pathway specifically focusing on the expression of ABCA1 and G1 in the lung post smoke exposure. After identifying an important role for ABCA1 and ABCG1 for the smoke effects on macrophage function the following experiments revealed a protective role for LXR agonists in the disease of emphysema with the up-regulation of ABC transporters and attenuation of MMP expression post cigarette smoke exposure.

Materials and Methods

Human Studies

The D'Armiento laboratory has stored de-identified human patient lung samples used to study the expression of ABC transporters (ABCA1 and ABCG1). The lung samples were classified into two categories: Normal, obtained from healthy humans, and COPD patients where the emphysema was quantified through lung morphometry. The mRNA was isolated from these samples and utilized for expression studies.

Cigarette Smoke Extract (CSE) Preparation

To prepare cigarette (CSE), the smoke from one cigarette (1.1 mg of nicotine, 15 mg of tar) was passed through 25 mls of phosphate buffered saline (PBS) (Mercer et al., 2004). The pH of the extracted solution was adjusted to 7.4 and then filtered (Mercer et al., 2004). CSE was added to the media at concentrations up to 5% (vol/vol) (Mercer et al., 2004).

Macrophage Cell Culture

In these experiments both mouse peritoneal and mouse bone marrow derived macrophages (BMdM) were utilized. Mouse peritoneal macrophages were isolated from peritoneal cavities of 3% thioglycolate injected mice on the 5th day from injection. To compare expression of genes in vitro and in vivo peritoneal macrophages were isolated from mice exposed to room air and cigarette smoke. For in vitro analysis cells were seeded on culture plates for 24 h and then treated with CSE. For in vivo analysis cells were collected from peritoneal cavity and mRNA was directly isolated. Mouse BMdMs were obtained from the following mice: ABCA1 fl/fl in a C57/BL6 background. Briefly, mice were first sacrificed by CO₂ asphyxiation followed by cervical dislocation. After sacrificing the mouse the legs were amputated and the excess muscle tissue was removed so as to completely expose the femur and tibia, which were cleaned carefully. These samples were left in sterile PBS for the duration of the procedure. The cavity bones were then flushed with 10 ml of PBS using 26G needle. The obtained cells were then counted (usually 100-150 million cells per mouse) and centrifuged for 5 min at 2600 rpm. Hematopoietic cells can be separated by plastic adhesion; therefore the cells were incubated in petri dishes containing DMEM culture media and supplemented with 10% FBS and 1% antibiotics). Non-adherent progenitor cells were then collected after 2 hours of incubation in culture media and then seeded at a concentration of 4 million cells/ml in fresh culture media, supplemented with L-cell conditioned media containing macrophage colony stimulating factor (M-CSF) at a concentration of 20%. Approximately after 7-10 days of plating the cells become fully differentiated into macrophages and were ready for subsequent experiments. After treatment the culture supernatants, proteins, and total RNA were collected for analysis.

For further analysis macrophages were stimulated with 5% CSE for 24 hours with or without 3 μM of LXR agonist (T0901317-Cayman Chemicals).

Animal Studies

Mice were chronically exposed to smoke in a specially designed chamber (Teague Enterprise, CA). 8 weeks old mice were smoke-exposed for 5 hours a day, 5 days a week for 10 days and 5 months. The total particulate matter (TPM) within the smoking chamber was regulated so that the mice receive a TPM of 100 mg/m³. TPM was determined by a gravimetric analysis of filter samples taken during the exposure period. Mice (C57BL/6, AKR/J strain) were smoke exposed for 10 days and 5 months and compared to room air exposed controls.

LXR Treatment.

Eight week-old AKR/J mice were utilized in the study and exposed to three different conditions: room air (n=8), cigarette smoke (n=8), cigarette smoke with additional treatment with LXR agonist (n=8). The LXR agonist was delivered in the diet and the concentration calculated based on the average mouse weight (30 g) and food consumption (6 g/day). Experimental food was prepared with a 0.015% LXR agonist (T0901317-Cayman Chemicals) in food (w/w) concentration corresponding to 30 mg/kg body weight. Both the control group and the custom drug diet were prepared by Research diets, Inc., New Brunswick, N.J. (Control diet (C11000)—Purina Rodent Chow 5001, Custom Diet (C13861)—Purina Rodent Chow 5001 with 0.015% LXR agonist (T0901317-Cayman Chemicals).

In addition to smoke exposure studies an evaluation of unexposed macrophage specific ABCA1 deficient mice was performed. Non-smoked 10 and 26 week-old ABCA1 Cre-LysM and ABCA1 Fl/fl mice were utilized to evaluate age related changes in lung inflammation.

Determination of Lung Compliance

To determine the pulmonary compliance of the lung, a closed chest model was utilized (Foronjy et al., 2005). Respiratory mechanics were measured using a flexiVent (SCIREQ) system (Foronjy et al., 2005). Pentobarbital was used for sedation and succinylcholine for paralysis. The mice then underwent neck dissection and tracheostomy followed by a full assessment of pulmonary mechanics in triplicate utilizing the flexiVent system (Foronjy et al., 2005). After the airway measurements were performed the animals were sacrificed by CO2 inhalation and further utilized for tissue, BAL and lung analysis.

Determination of Lung Inflammation

The BAL fluid was collected by perfusing the lungs with 2 ml of phosphate buffered saline (PBS). The samples were then centrifuged to pellet down the cells and re-suspended in 300 μl of PBS. To obtain the total cell count, cells were counted using a hematocytometer for which, 10 μl of the sample was utilized. To obtain the differential cell count, the slides were prepared using a cytopsin. 150 μl of sample was taken to prepare slides. These slides were stained using Hematoxylin and Eosin (H&E) and a number of regular and foamy like macrophages and lymphocytes counted under the microscope. A foamy like phenotype (Hirama et al., 2007) was identified by examining the sequential increase in macrophage size. Lipid staining was not performed.

Histology

Histological evaluation of the inflammation was performed on the left lungs which were fixed using 10% formalin. Fixed tissue was stained with H&E and Trichrome and mean linear intercept (MLI) determined as described previously (Foronjy et al., 2008; Foronjy et al., 2005; Foronjy et al., 2001; Foronjy et al., 2010; Foronjy et al., 2008a; Foronjy et al., 2006; Foronjy et al., 2003; Geraghty et al., 2013).

Real Time (RT) PCR

Total RNA was extracted from specimens of lung tissue 0.3 cm³ in size with the use of the RNeasy kit (Qiagen, Germantown, Md.) (Goldklang et al., 2012). The RNA was then further processed to obtain cDNA, which was used for Real Time (RT) PCR analysis. TaqMan gene expression assays (Applied Biosystems, Carlsbad, Calif.) were performed to assess gene-transcript levels with the use of an ABI Prism 7900HT Sequence Detection System (Applera, Foster City, Calif.) (Goldklang et al., 2012). The following primers were used to check the respective gene expression: ABCA1 (Hs01059118_m1), ABCG1 (Hs00245154_m1), MMP-9 (Mm00442991_m1), IL-1β (Mm00434227_g1), TNF-α (Mm00443260_g1) and GAPDH (4352932E) from TaqMan Gene Expression Assays, Applied Biosystems, CA. β-actin was used as a control since it is a housekeeping gene.

Western Blotting

The lungs of mice (-10 mg) were homogenized in 1 ml of Radioimmunoprecipitation assay (RIPA) buffer, and centrifuged at 14,000 g for 10 min. The protein concentration of each sample was measured using a Bradford reagent. The required amount of each sample was then used for western blot analysis, so that the concentration was equal for all samples. Proteins were first separated on the basis of molecular weight via sodium dodecyl sulphate polyacrylamide gel electrophoresis (SDS-PAGE). The same gel was then transferred onto a nitro cellulose membrane with the help of electric current (or electroblotting) that transfers the protein from polyacrylamide gel to the membrane. This membrane was then incubated in the solution containing primary antibodies, specific for the proteins of interest, ABCA1 and ABCG1, followed by the use of a secondary antibody that allowed the detection of protein by chemiluminescent. ABCA1 (pAb anti-ABCA1 Antibody-NB400-105) and ABCG1 (pAb anti-ABCG1 Antibody-NB400-132) antibodies were used, following the manufacturer's (Novus Biologicals) instructions.

Cholesterol Efflux Assay

Mouse macrophages were placed in a 0.5 ml/well of DMEM with fatty acid free BSA (0.2%) and antibiotics (P/S) and loaded with ³H-cholesterol (1μCi/ml) together with acetylated LDL (50 μg/ml) for 24 hours (Yvan-Charvet et al., 2007). The macrophages were then washed two times with the media, followed by treatment with the cigarette smoke components (CSE) for 2 hours. The efflux media was then added, containing serum (2.5%), ApoAI (25-50 μg/ml) and HDL (25 μg/ml) in addition to the respective smoke components. The media was collected after 6 hours of incubation and the cells were lysed in 0.5 ml of NaOH (1M). The efflux was calculated as follows: [cpm media/(cpm media+cpm lysate)]×100 (Yvan-Charvet et al., 2007).

Statistical Analysis

Statistical analysis of the data obtained was performed using the unpaired two-tailed Student's T-test when 2 groups were being compared. In the case of a three or more group comparison one-way ANOVA followed by the Bonferroni post hoc test was performed using GraphPad Prism software (Goldklang et al., 2012).

Results

Cigarette Smoke Blocks ABC Transporter Expression in Macrophages and Impairs Cholesterol Efflux.

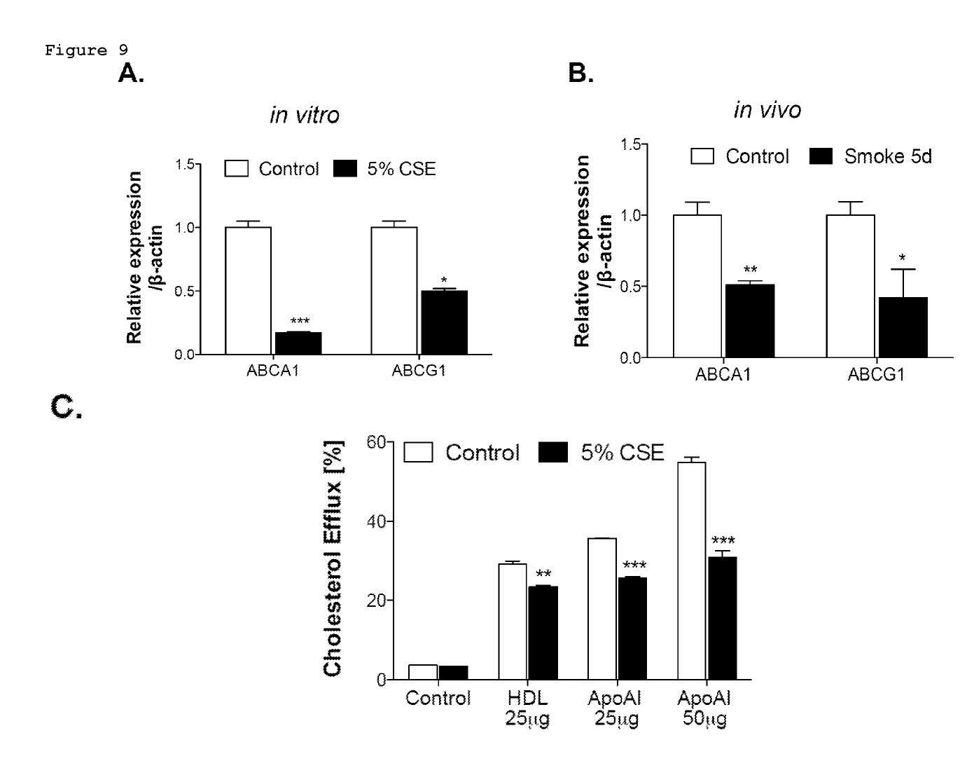

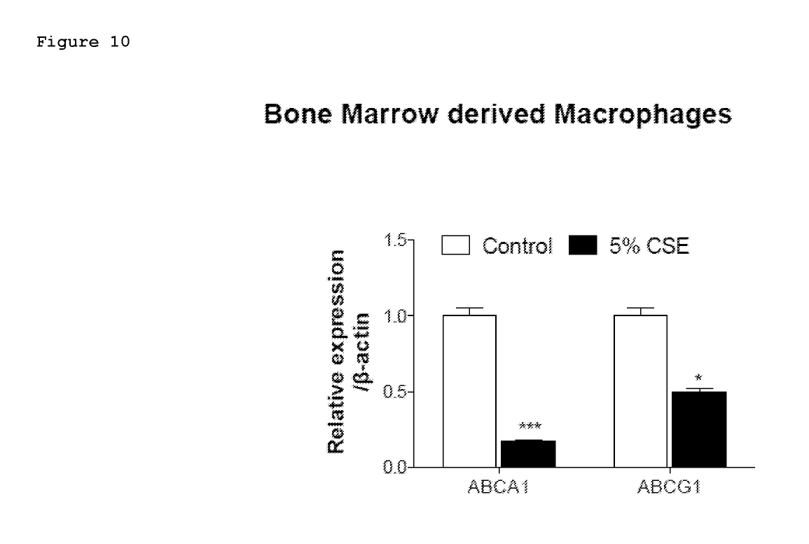

To determine the consequence of cigarette smoke exposure on active cholesterol transport in macrophages peritoneal macrophages were exposed to cigarette smoke extract (CSE) or cigarette smoke in vivo. A reduction in the expression of the two main active cholesterol transporters ABCA1 and ABCG1 were observed under smoke exposure conditions (FIG. 9A., B. respectively). Furthermore these changes were accompanied by a significant impairment of the cholesterol efflux potential of these macrophages in vitro towards HDL and much more prominent towards ApoAI which is the main acceptor of cholesterol effluxed by ABCA1 (FIG. 9C.). These findings demonstrate that cigarette smoke itself without lipid exposure is able to modulate the efflux pathway suggesting that cholesterol transport plays a possible role in cigarette smoke related lung diseases.

Cigarette Smoke Downregulation of ABC Transporters Correlates with Macrophage Activation and an Increased Inflammatory Response In Vitro.

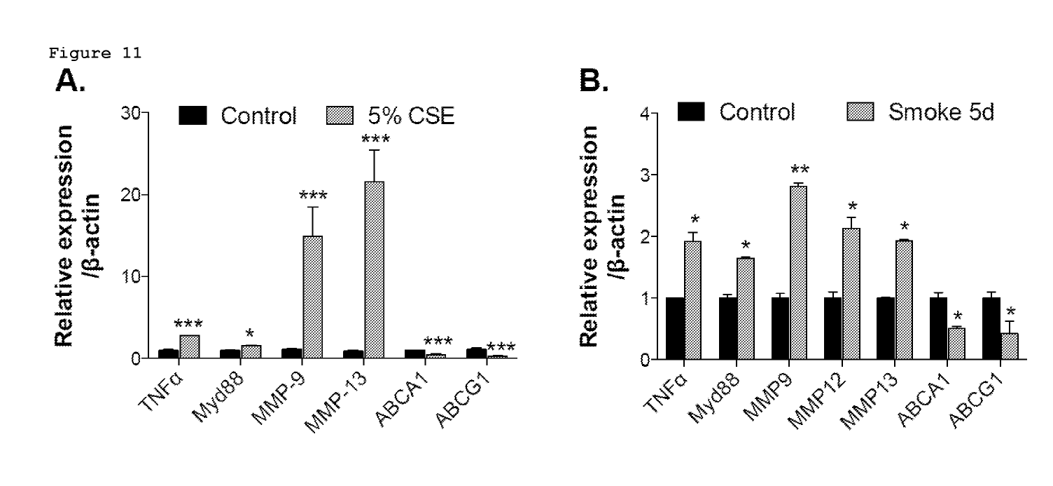

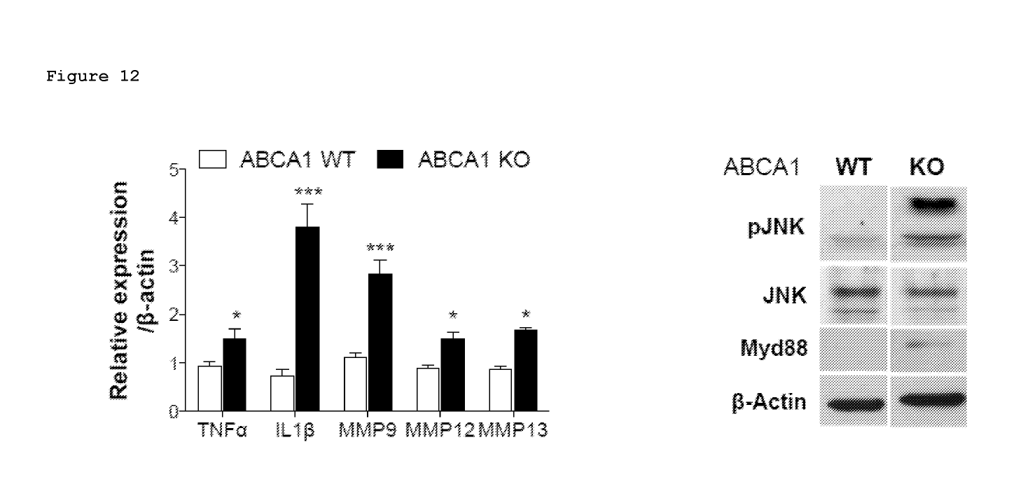

Interestingly, the downregulation of ABC transporters in macrophages correlated with the increased expression of genes consistently dysregulated under smoke exposure conditions. Genes that are known to modulate inflammation (TNFα, Myd88) were upregulated and genes encoding for destructive matrix metalloproteases (MMP-9, MMP-12 and MMP-13) critical in the disease of emphysema were increased in macrophages under smoke exposure conditions when ABCA1 and ABCG1 were downregulated in vitro (FIG. 11 A.) and in vivo (FIG. 11B.).

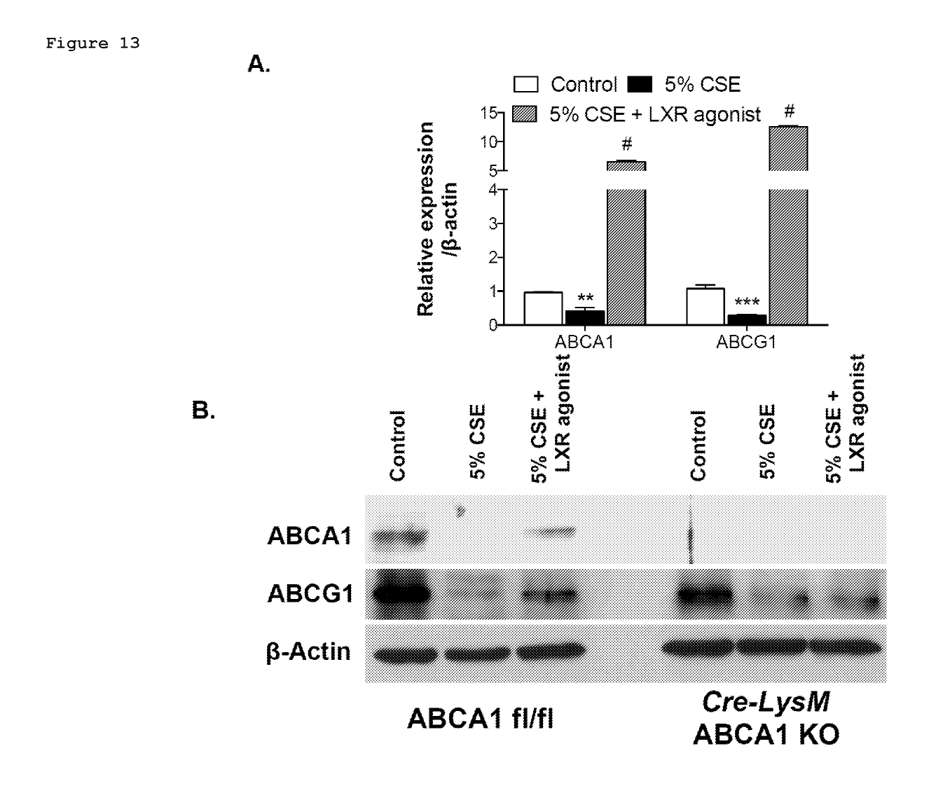

To further explore the relationship between smoke exposure and the regulation of ABC transporters bone marrow derived macrophages were isolated from mice lacking specific expression of ABCA1 in macrophages (FIG. 13.). Additionally, ABC transporter expression was re-established by treating the cells with an LXR agonist (T0901317-Cayman) (FIG. 13.). As expected both at the mRNA and protein level CSE inhibited the expression of both ABCA1 and ABCG1 (FIG. 13A) with ABCA1 completely blocked at the protein level (FIG. 13B). Expression of both transporters was maintained at baseline levels when cells were treated with cigarette smoke and the LXR agonist (FIG. 13A-B). Importantly, in the ABCA1 KO macrophage cigarette smoke did not induce ABCA1 expression but cigarette smoke still downregulated and reduced the level of ABCG1 as expected (FIG. 3B.).

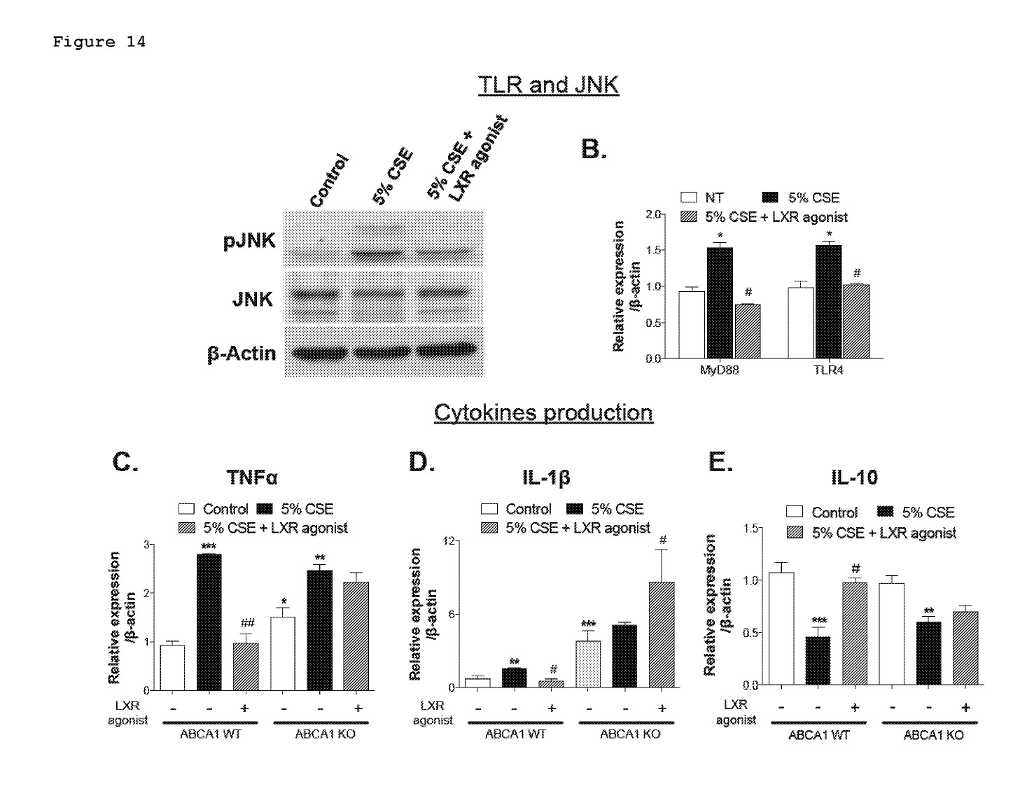

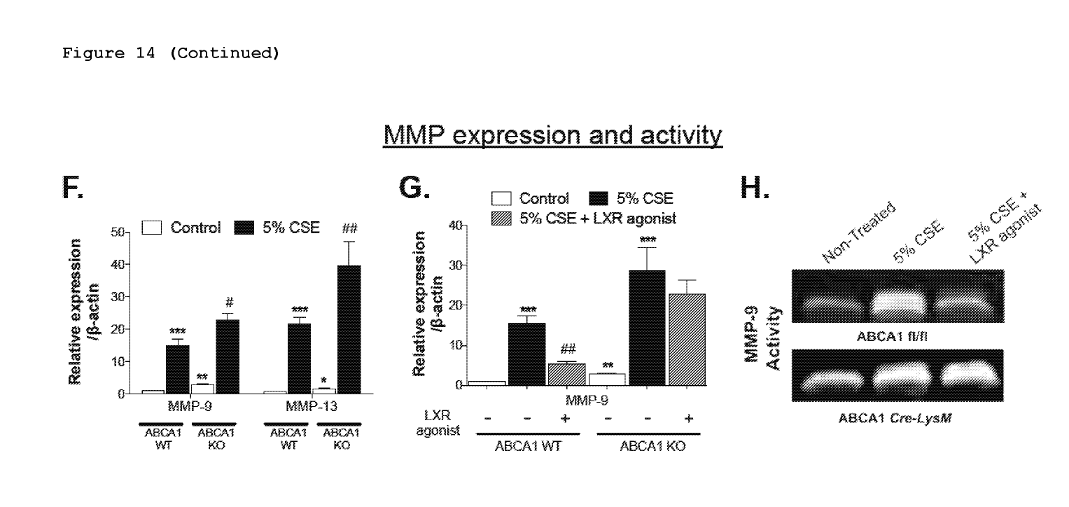

After documenting that the loss and gain of function approach (FIG. 13.) was functional studies were performed to examine the effect of ABC loss on the cigarette smoke induced inflammatory and proteases response in vitro. Initially mouse macrophages were exposed to 5% CSE and treated with the LXR agonist. Interestingly LXR agonism inhibited cigarette smoke induced phosphorylation of JNK and ERK (FIG. 14A.) and mRNA expression of TLR4/Myd88 (FIG. 14B.). Phosphorylation of JNK and ERK as well as increased expression of TLR4/Myd88 is within the pathway of cigarette smoke activation of inflammatory cytokines and MMPs. Subsequently, ABCA1 WT and KO mouse macrophages were exposed to 5% CSE with and without LXR agonies treatment. Treatment of macrophages with 5% CSE induced the upregulation of IL-1β and TNFe and downregulation of IL-10 which was reversed back to baseline with LXR agonist treatment (FIG. 14C-E.) in macrophages isolated from the ABCA1 WT mice. Importantly, the LXR agonist was not able to reverse the induction of cytokines by 5% CSE in the ABCA1 macrophage knockout mice (FIG. 14E-C.). These findings suggest that the ABCA1 transporter plays a significant anti-inflammatory role in the cigarette smoke induced activation of macrophages. Most notably, the upregulation of MMP-9 and MMP-13 by CSE was potentiated in the macrophages lacking ABCA1 (FIG. 14F.). In addition, treatment with the LXR agonist reduced the mRNA and activity of MMP-9 only in the presence of the ABCA1 transporter (FIG. 14G-H.)

Cigarette Smoke Induced Downregulation of ABC Transporters Correlates with Macrophage Activation and an Increased Inflammatory Response In Vivo.

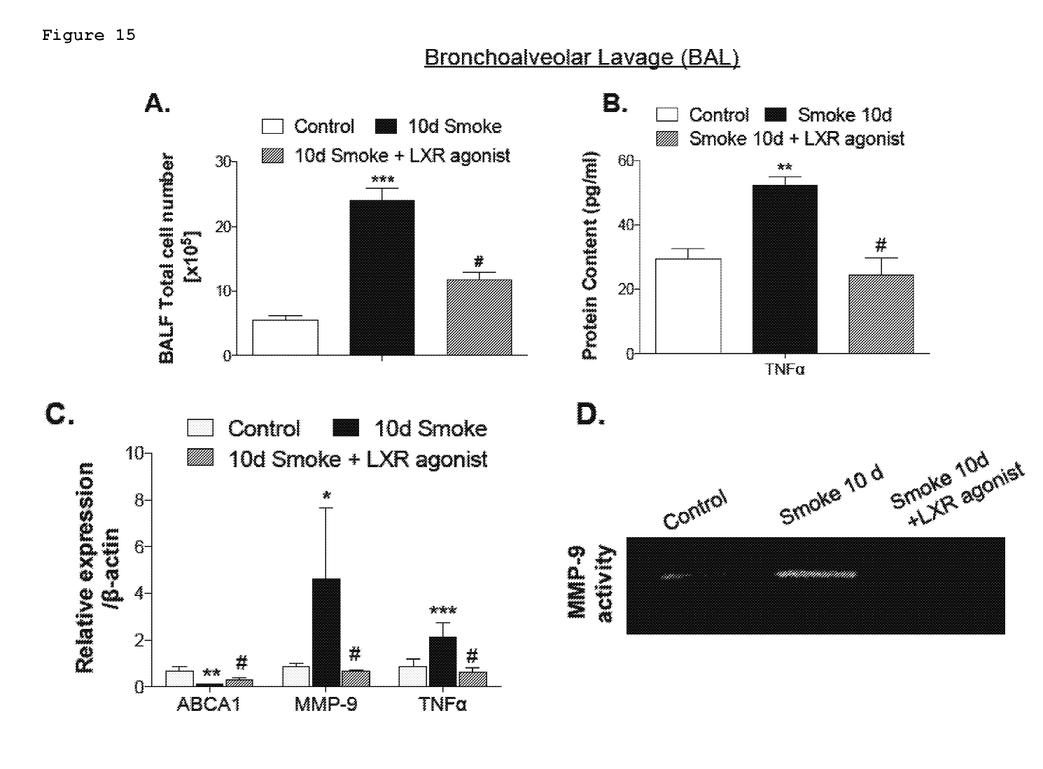

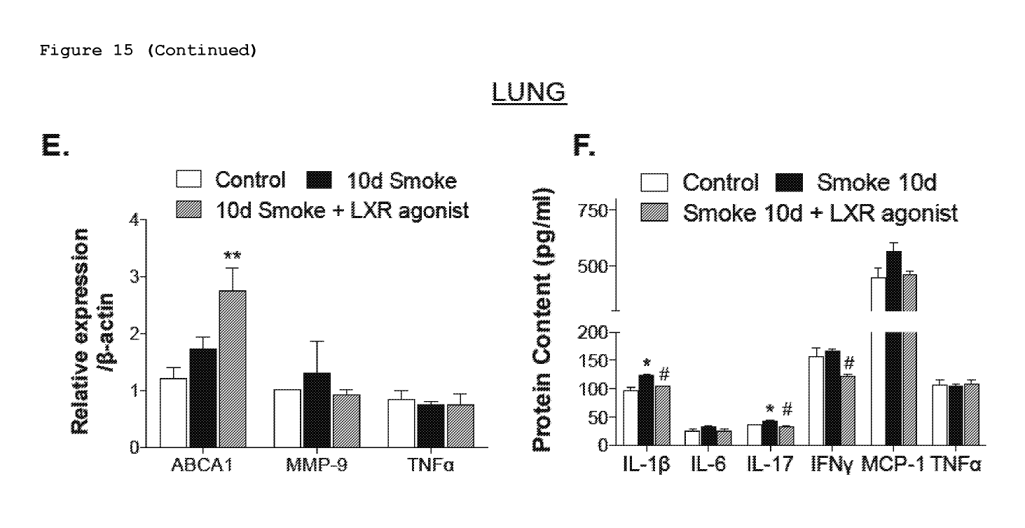

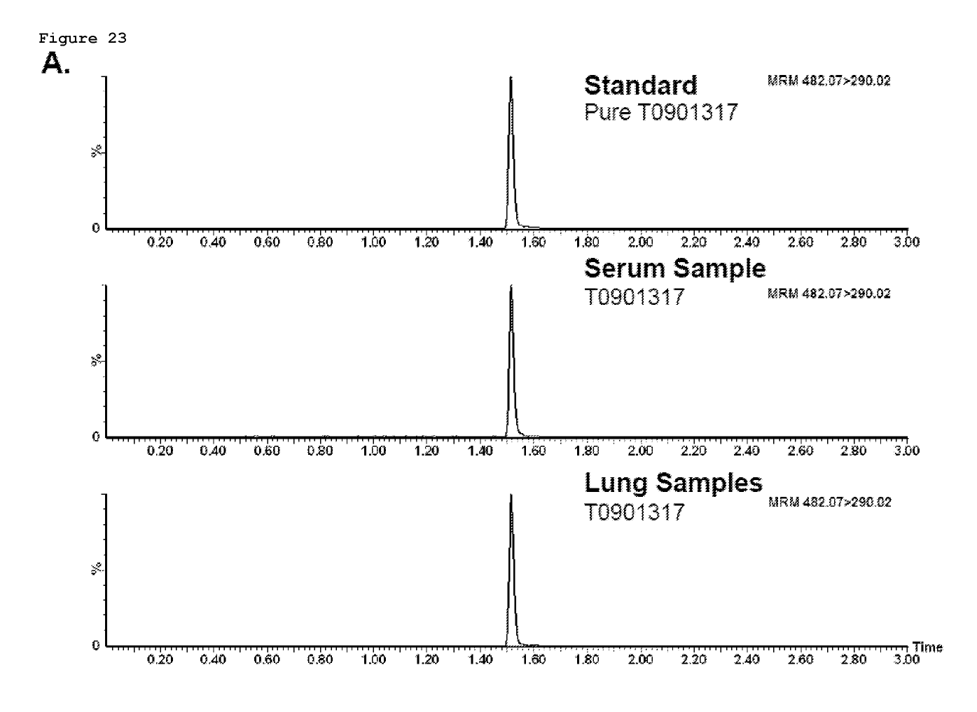

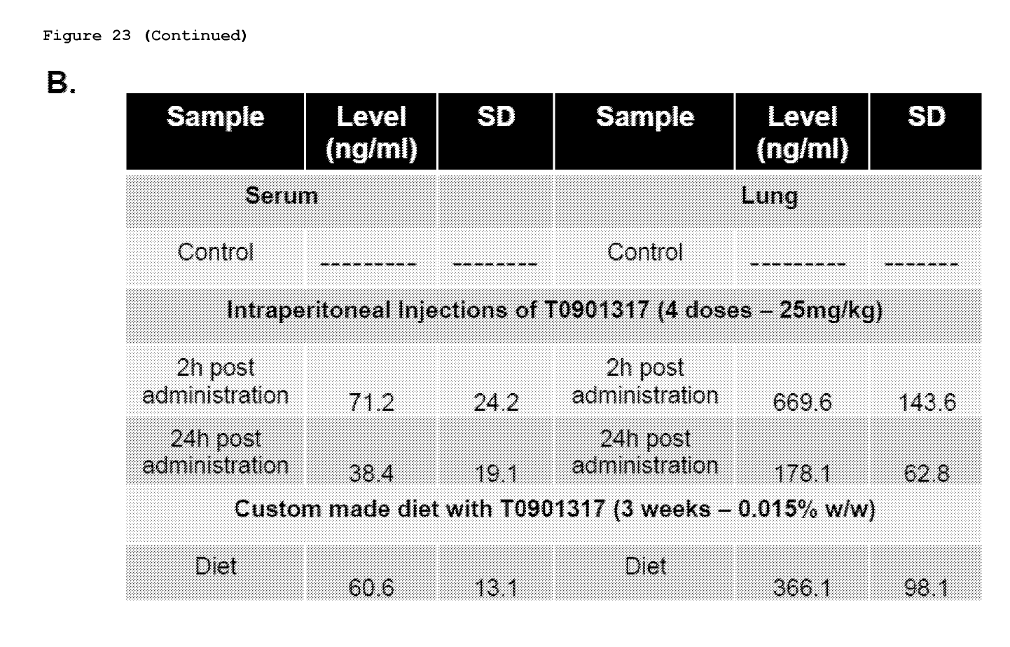

To determine if the mechanisms identified in vitro contributed to changes in smoke induced lung inflammation in vivo, cigarette smoke studies were performed in mice. The treatment of mice exposed to cigarette smoke in an acute 10 day model with 25 mg/kg of intraperitoneal (IP) injections of an LXR agonist daily during the last 4 days of exposure was examined. LXR agonist treatment successfully increased the level of ABCA1 in BAL (FIG. 15C.) and was detected in the lungs after IP administration in this model (FIG. 23.). Treatment with the LXR agonist was capable of blocking the already established inflammation and decreased the total inflammatory cell counts (FIG. 15A.). Furthermore, the cigarette smoke induced TNFα protein expression in the BAL was abrogated in mice treated with the LXR agonist (FIG. 15B.). In addition, LXR agonist treatment decreased the mRNA levels of TNFα (FIG. 15C.) while inducing ABCA1 expression in alveolar macrophages from BAL (FIG. 15C.). Most importantly, cigarette smoke induced MMP-9 expression and activity in the BAL were inhibited by the treatment with the LXR agonist (FIG. 15C-D.), which correlated with inhibition of inflammation and the inflammatory driven increase in TNFα. LXR agonist treatment of mice acutely exposed to cigarette smoke also inhibited the smoke induced increase in IL-1β, IL-17, IFNγ and MCP-1 in the lung tissue (FIG. 15F.).

LXR Agonism Reverses Cigarette Smoke Induced Inflammation and Emphysema Development in Chronic Model of Empysema in Mice.

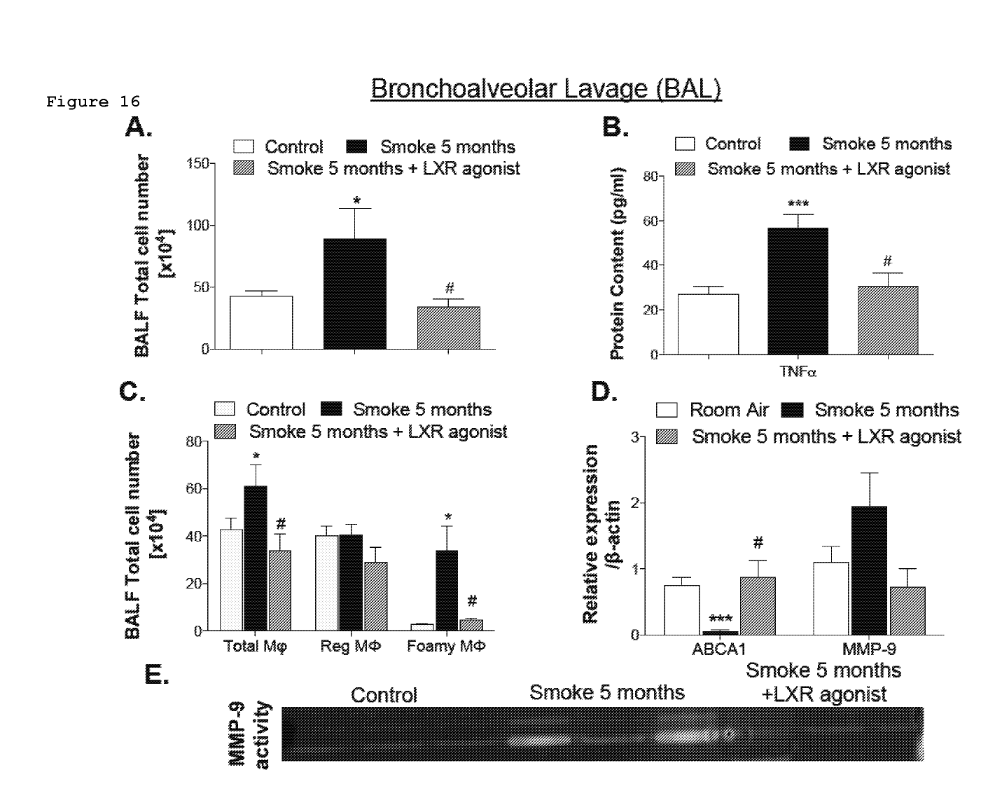

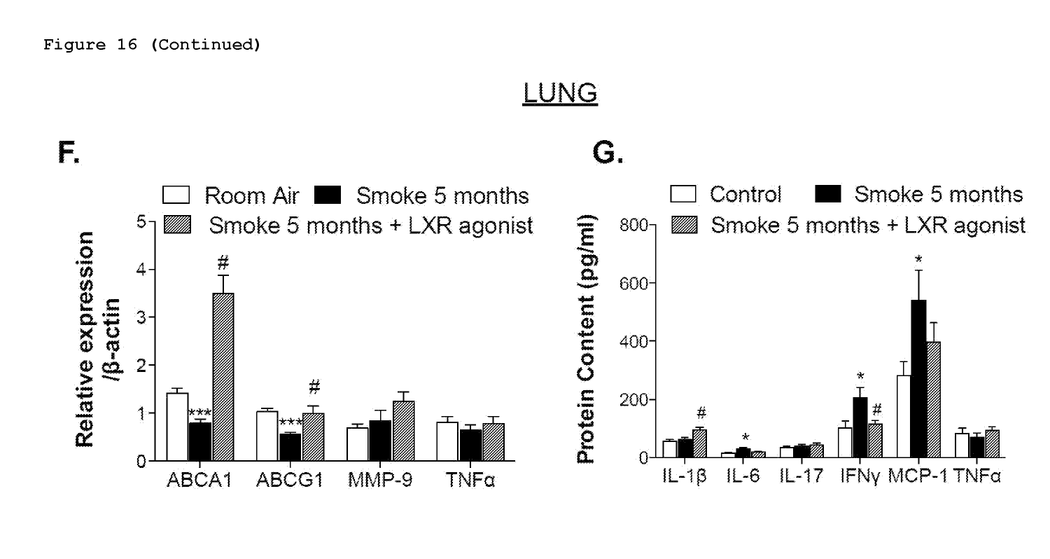

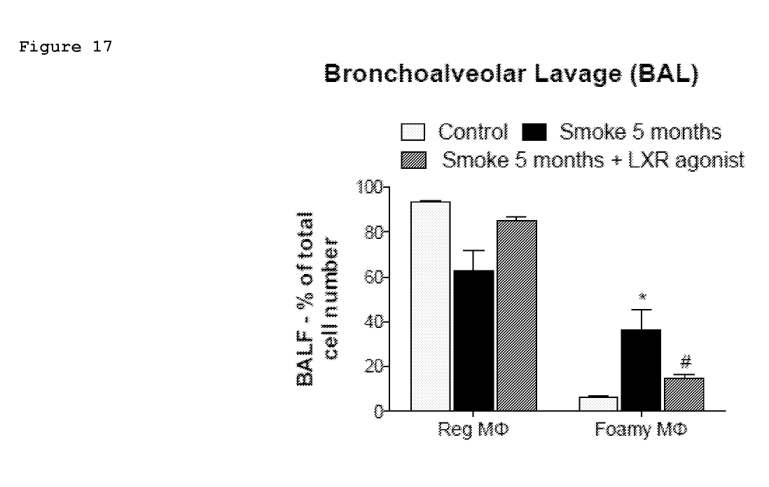

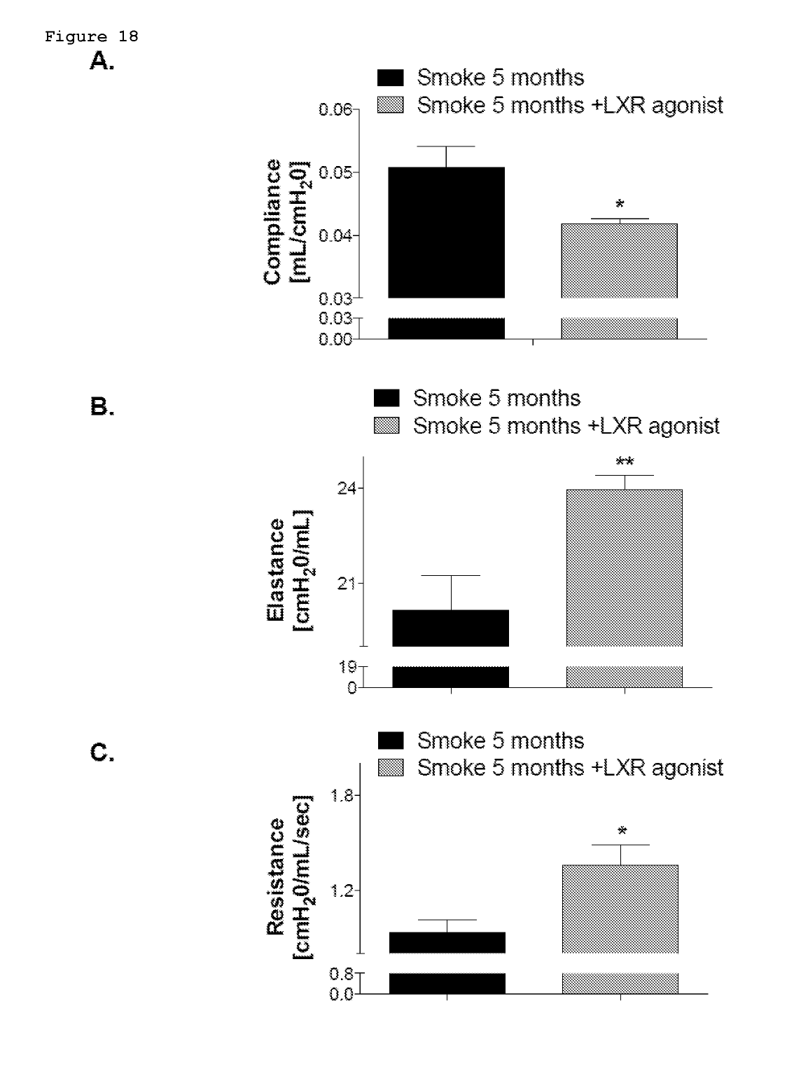

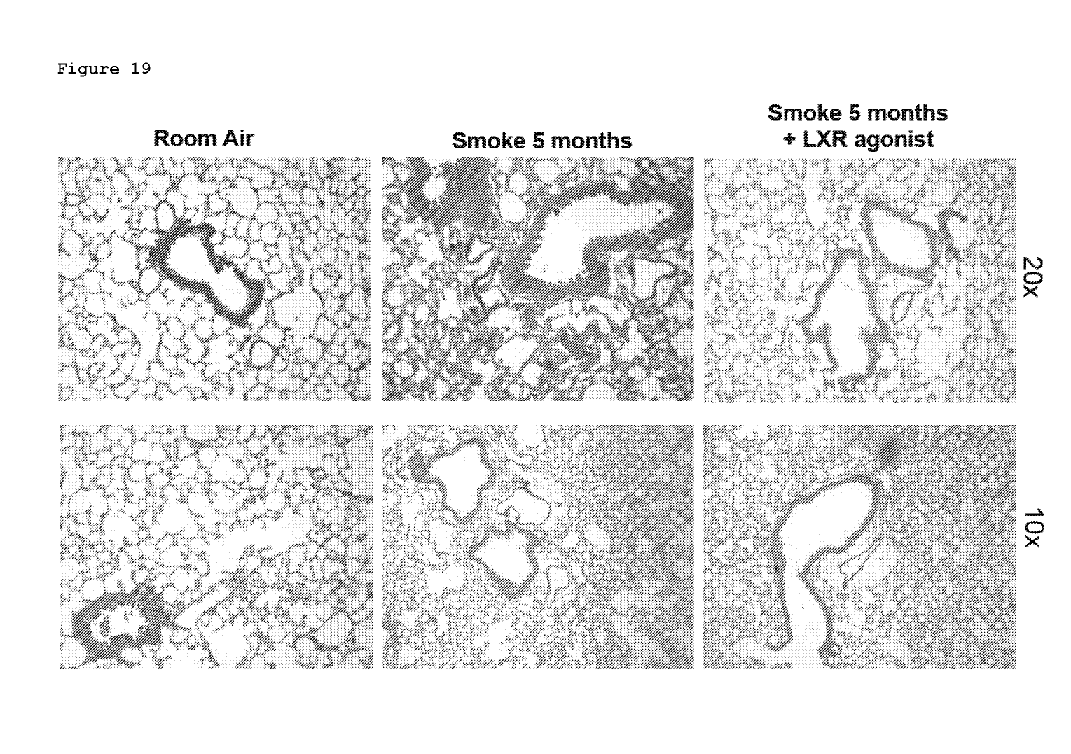

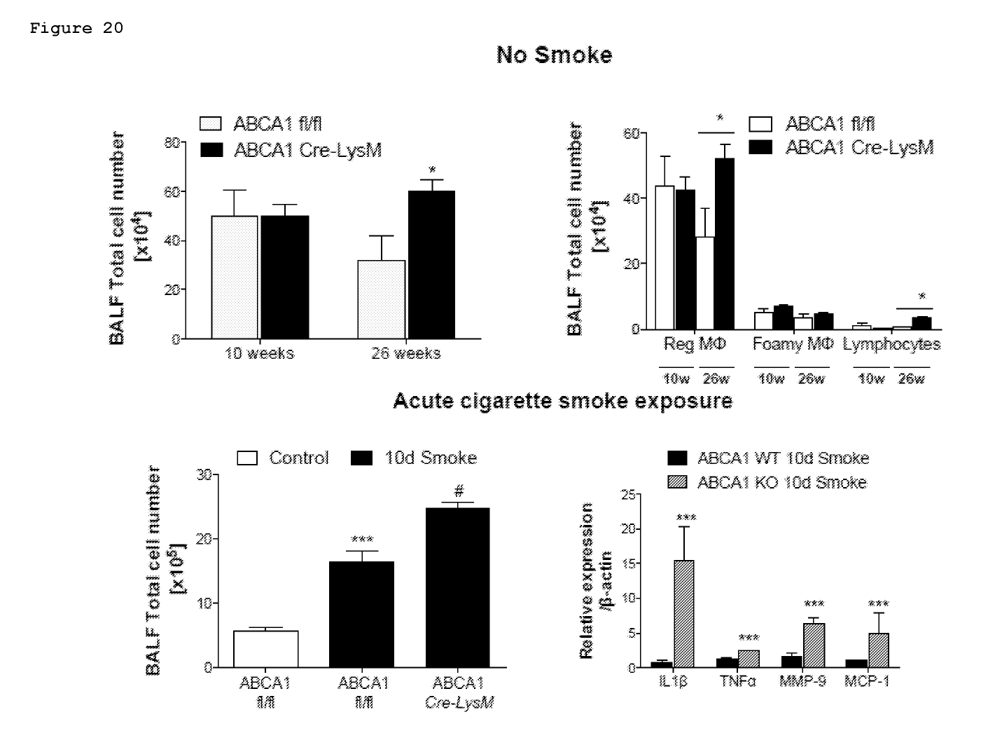

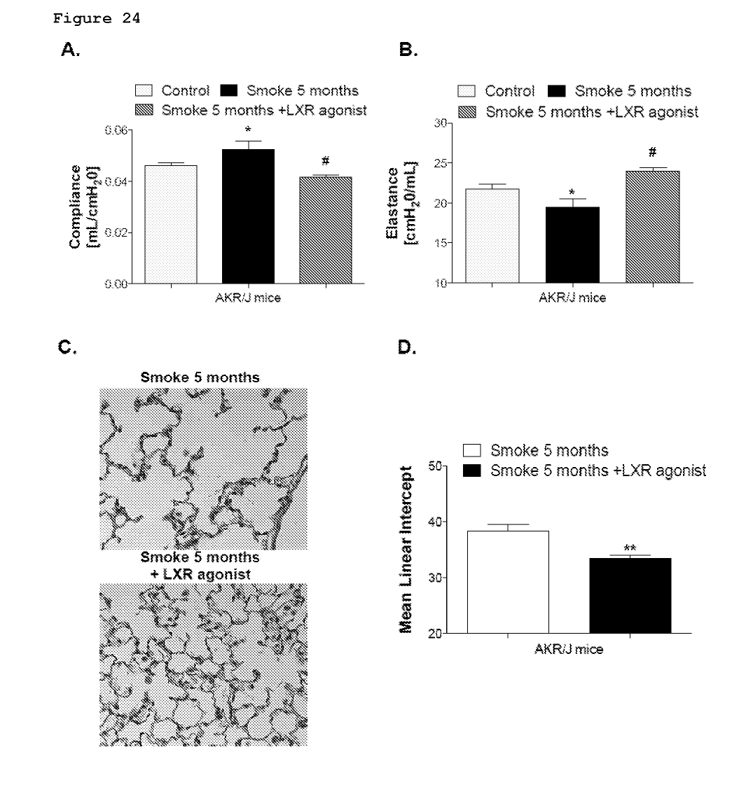

In order to determine if the mechanisms identified above contribute to inflammation and tissue destruction observed in a chronic cigarette smoke exposure model, long term cigarette smoke studies were then performed in mice. Mice exposed to cigarette smoke for 5 months were treated with an LXR agonist in the diet (0.015% w/w) that corresponded to 30 mg/kg. LXR agonist treatment successfully increased the level of ABCA1 in both the BAL and lung tissue (FIG. 16D., F) and LXR agonist was detected in the lungs after IP administration in this model (FIG. 23.). Treatment of mice exposed to cigarette smoke in the chronic 5 months model significantly decreased the total inflammatory cell counts (FIG. 16A.). Furthermore cigarette smoke induced protein levels of TNFα in the BAL were abrogated in the mice treated with the LXR agonist (FIG. 16B.). Differential cell counts demonstrated that the BAL consisted mostly of alveolar macrophages and the treatment with the LXR agonist significantly reduced the alveolar macrophage population exhibiting a foam cell-like phenotype (FIG. 16C.). In addition LXR agonist treatment restored the levels of ABCA1 that were downregulated secondary to cigarette smoke (FIG. 16D.). mRNA levels of MMP-9 in the BAL cells of the chronic smoke exposure model were not significantly altered, however the smoke induced MMP-9 activity in the BAL was attenuated with the LXR agonist treatment (FIG. 16E.) which correlated with the inhibition of inflammation and the inflammatory driven increase in TNFα. LXR agonist treatment of mice chronically exposed to cigarette smoke inhibited the smoke induced increase in IFNγ and MCP-1 in the lung (FIG. 16G.). No significant changes in mRNA expression were found for MMP-9 and TNFα in the lungs (FIG. 16F.). Long term treatment of mice chronicly exposed to smoke with the LXR agonist improved lung function as measured by a decrease in lung compliance (FIG. 24A) and an increase in lung elastance (FIG. 24B.). Mean linear intercept quantification also revealed, that LXR agonise treatment preserved the lung structure and blocked emphysema development in the mice exposed to cigarette smoke for 5 months (FIG. 24C-D.).

ABC Transporters Downregulation in Lungs of Patients with COPD

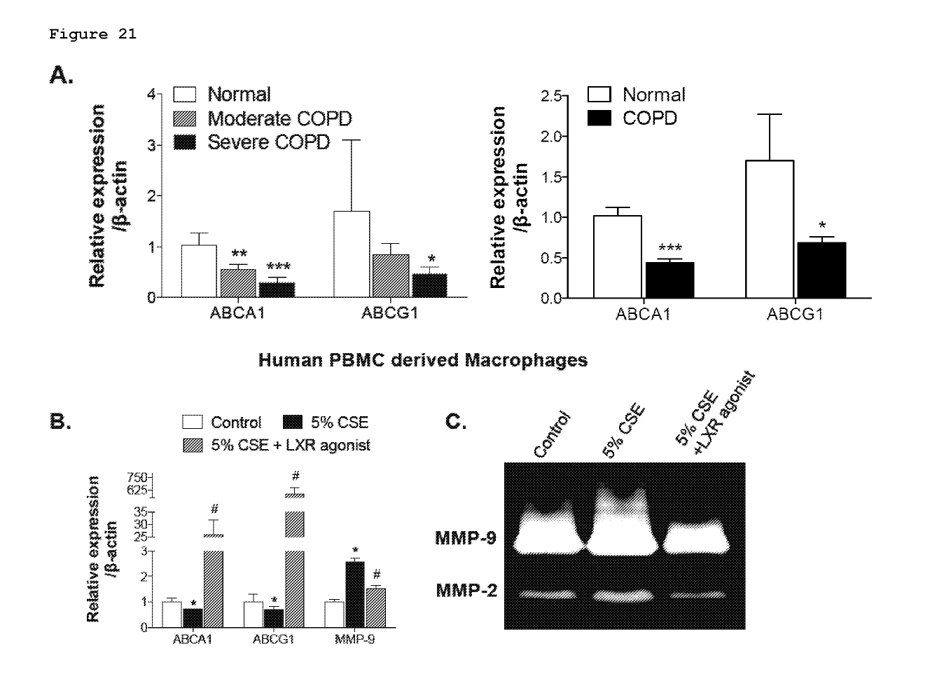

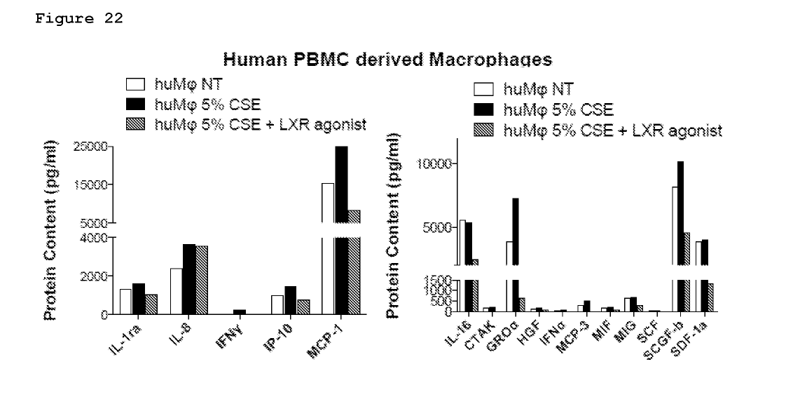

To confirm the relationship of these findings to the human disease, the lung mRNA expression of ABCA1/G1 from patients with or without COPD was evaluated. Interestingly, downregulation of ABC transporters, particularly ABCA1, was observed in the lungs of patients diagnosed with COPD (FIG. 21A.). In addition, human macrophages that underwent differentiation to macrophages from PBMC and were exposed to 5% CSE with or without LXR agonist treatment (FIG. 21B-C.) were found to downregulate ABC transporters secondary to cigarette smoke exposure and the ABC transporter levies could be restored by treatment with an LXR agonist (FIG. 21B.).

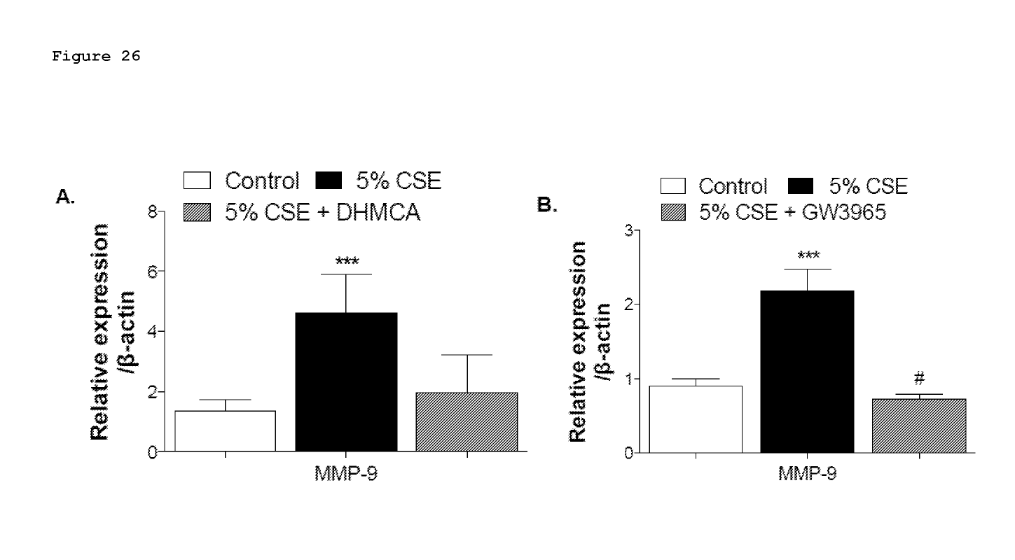

Furthermore exposure to 5% CSE increased MMP-9 expression and activity which could subsequently be inhibited by treatment with an LXR agonist (FIG. 21B-C.). Two additional LXR agonists, DHMCA and GW3965, were also able to block MMP9 expression after smoke exposure (FIG. 26).

LXR Agonism Regulates Accumulation of Ceramides in Chronic Model of Emphysema in Mice.

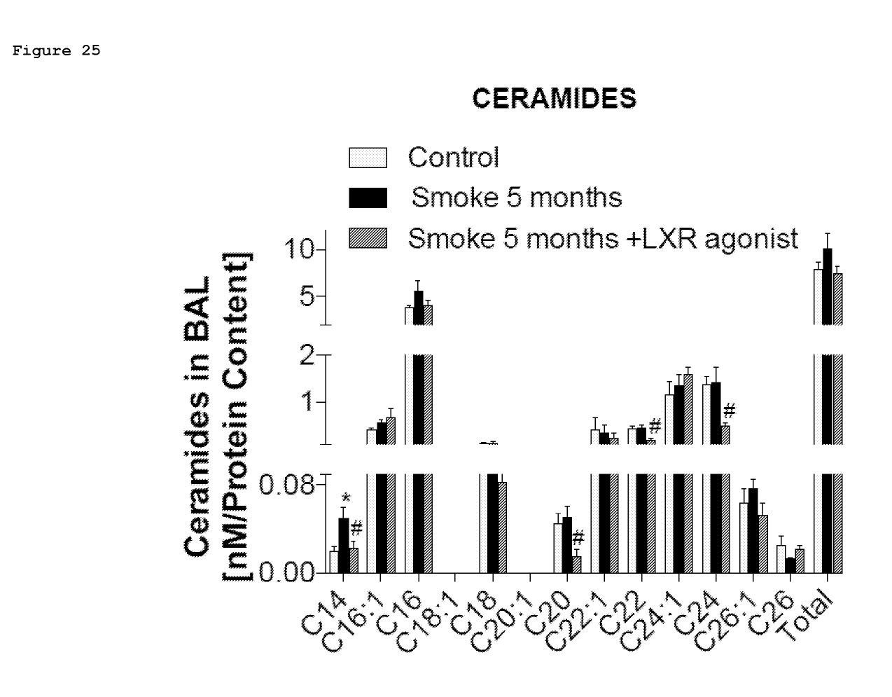

Recent studies have shown that ceramides are a marker of emphysema and may play a causative role in the development of the disease (Petrache et al., 2005; Petrache et al., 2013; Petrusca et al., 2010). Since changes in ABC transporter expression could potentially alter ceremide levels mass spectrometry analysis was performed on BAL fluid from mice exposed to room air and cigarette smoke for 5 months with or without LXR agonist treatment. Interestingly, the total level of ceramides in the BAL increased in smoke exposed mice and was reduced by treatment with the LXR agonist (FIG. 25). Furthermore, C14 and C16 have been reported to be highly upregulated ceramides in patients with emphysema (Petrache et al., 2005). In these studies the observed cigarette smoke induced increase in C14 and C16 ceramides was reduced by treatment with the LXR agonist (FIG. 25) and correlated with the improvement in lung function and structure (Pleurae 24C)

Discussion

The described studies document the effect of cigarette smoke on ABCA1 and ABCG1 dependent cholesterol transport molecules in macrophages, linking these transporters with cigarette smoke induced pulmonary inflammation and induction of detrimental MMPs in the lung particularly in macrophages in relation to cigarette smoke induced emphysema. Dowregulation of ABC transporters due to cigarette smoke in macrophages in vivo and in vitro, in animal model and human disease accompanied by the increase in inflammation and MMP-9 activity suggests that cigarette smoke regulation of ABC transporters controlled inflammation and proteinase activity is a key player in emphysema progression.

Deficiency in both ABCA1 and ABCG1 in macrophages prevents the protective role of HDL and leads to increased secretion of inflammatory cytokines (Buist et al., 2007). Interestingly, ABCA1 and ABCG1 KO mice manifesting abnormal cholesterol efflux exhibit pulmonary inflammation (Baldan et al., 2008; Bates et al., 2005). Macrophages from these mice exhibit an increase in the expression of inflammatory and oxidative stress genes via TLR signaling, suggesting a link between alterations in cholesterol efflux and lung inflammation through TLR signaling (Yvan-Charvet et al., 2010; Yvan-Charvet et al., 2010a; Yvan-Charvet et al., 2008). In fact, the excessive presence of foamy macrophages merged with the up regulation of MMP-9 and MMP-12 was demonstrated in cigarette-smoke induced emphysema in mice (Hirama et al., 2007). Induction of inflammatory signaling pathways, mobilization of macrophages to the lung, and tissue destructive matrix metalloproteinase (MMP) expression in macrophages are all hallmarks of cigarette smoke related pulmonary diseases such as emphysema or COPD. Prior studies in the D'Armiento laboratory demonstrated that hypercholesterolemia contributes to emphysema development in ApoE KO mice, with mice demonstrating increased inflammatory cells in the lung (Goldklang et al., 2012). The combination of the prior work with the above described study suggests that modifications of lipids can alter inflammation and macrophage activation playing an important role in disease pathogenesis. Cigarette smoke induced downregulation of ABCA1 potentiates both the inflammatory and proteolytic activity of macrophages and could be very important factor in progression of emphysema.

Both the in vivo studies combined with the cell culture experiments demonstrate an important association between cigarette smoke exposure and cholesterol mediated pathways in macrophages regulated by ABC transporters. Importantly, modulation of these pathways through manipulation of ABCA1 with several LXR agonists effectively blocked smoke induced inflammation suggesting that targeting this pathway has novel therapeutic potential for the treatment of COPD.

Based on the above data, smoke regulation of cholesterol transport, inflammation and lung tissue destruction by MMPs all appear to be mechanistically linked to changes in transporter expression, playing a role in the increased risk of emphysema development in smokers and potentially presenting a therapeutic target. Thus, the work described herein fills a critical gap in the existing literature and provides insight into how tobacco smoke regulation of ABC transporters affects emphysema development.

The in vitro and in vivo studies clearly demonstrate that LXR agonists, which protect the levels of ABC transporters under smoke exposure conditions, can shield the lung from the destructive effects of cigarette smoke. Both short term and long term smoke exposure studies document the blunted inflammatory and proteolytic response in animals exposed to smoke and treated with an LXR agonist. Most importantly, the LXR agonist attenuation of inflammation was demonstrated to protect the animals from the destructive functional and structural changes seen in the lung secondary to chronic smoke exposure. Therefore, the LXR agonists can be seen as potential novel therapeutic targets in COPD.

### EXAMPLE 4

### LXR agonists reverse cigarette smoke induced emphysema.

Human subjects suffering from cigarette smoke inducted emphysema are administered an LXR agonist or placebo for 6 months. The LXR agonist is administered as a monotherapy.

By 6 months, the emphysema in subjects receiving the LXR agonist is at least partially reversed. In some instances, the emphysema is completely reversed. In contrast, human subjects not administered the LXR agonist do not show reversal of emphysema.

Discussion

Highlights
Liver X receptors (LXR) are transcription factors that are largely considered to be cholesterol ‘sensors’, that when activated leads to decrease plasma cholesterol (Viennois et al., 2011).
LXRs are also known to suppress inflammatory signaling in macrophages (Joseph et al., 2002).
Macrophages secret matrix metalloproteases (MMPs) (Webster et al., 2006), which are the primary enzymes in the destruction of lungs in emphysema.
LXR agonists are commercially available (T0901317, GW3965).
In the United State approximately 4.7 million people have been diagnosed with emphysema (Center for Disease Control and Prevention, 2013).

Aspects of the present invention relate to:
Targeting a new signaling pathway to reduce the destructive effects of MMPs in the lung.
Utilizing LXR agonists to treat emphysema and COPD, including but not limited to cigarette smoke induced emphysema and COPD.
Providing in vitro data demonstrating a reduction in smoke-induced destructive MMPs and proinflammatory cytokines by LXR agonists.

Additionally, the present disclosure provides in vivo data demonstrating that LXR agonists can resolve pulmonary inflammation in a mouse model.

Introduction

A protease/anti-protease imbalance is a dominant paradigm explaining the pathogenesis of cigarette smoke-induced emphysema where cigarette smoke exposure promotes repeated proteolytic injury to the extracellular matrix. The evolution of this paradigm is intimately linked to the recognition of the macrophage as a major effector cell in the response to cigarette smoke; the quantity of macrophages within the alveolar walls correlates with emphysema severity (Finkelstein et al., 1995). Indeed, studies examining human alveolar macrophages and bronchoalveolar lavage fluid (BALF) have confirmed that a protease/antiprotease imbalance exists in cigarette smoke-induced emphysema or COPD (Shapiro et al., 1999; Pons et al., 2005; Finlay et al., 1997; Finlay et al., 1997a; Finlay et al., 1993b). In particular, macrophages from the BALF of patients with emphysema exhibit increased mRNA transcripts of MMP-1, MMP-13, MMP-12 and MMP-9 associated with increased collagenase and elastolytic capacity when compared with macrophages from normal subjects (Finlay et al., 1997a; Finlay et al., 1993b; Woodruff et al., 2005). The inventors and others have documented the importance of MMPs in emphysema development (Woodruff et al., 2005; Foronjy et al., 2008; D'Armiento et al., 1992; Hautamaki, 1997).

Cigarette smoke is also associated with a significant increase in the secretion of cytokines such as TNFα (Yoshida et al., 2007; Thomson et al., 2012; Letuve et al., 2008), IL-1 (Churg et al., 2009; Couillin et al., 2009; Doz et al., 2008), IL-8 (Karimi et al., 2006), IL-13 (Zheng et al., 2000), and IFNγ (Wang et al., 2000) through the induction of endogenous danger signals, sensed by pattern recognition receptors such as Toll-like receptors (TLR). Signaling through. TLR4 triggers activation of macrophages leading to lung inflammation and a tissue destruction progru via TLRs/MMP (Geraghty et al., 2011) or IL-1/Myd88 (Couillin et al., 2009; Doz et al., 2008; Karimi et al., 2006) ultimately resulting in the release of MMPs from alveolar macrophages. The augmentation of lung inflammation, which results in a significant increase in protease activity, is a crucial step in the resultant alveolar destruction that is characteristic of emphysema. In addition IL-1, TNFα, and the TLR/Myd88 pathway have been shown to be important regulators of sphingolipid production such as ceramide and sphingomyelin (Holland et al., 2008) both of which have recently been implicated in emphysema pathogenesis (Petrache et al., 2005).

Cigarette Smoke Induced Inflammation Links with Impaired Cholesterol Transport

Multiple organ systems are affected by smoking, with smokers displaying higher susceptibility and increased severity of cardiovascular disease (Barnoya et al., 2005; Freund et al., 1993; Howard et al., 1998; Milei et al., 1998). A common feature of both emphysema and atherosclerosis is inflammation originating from the infiltration of macrophages and lymphocytes into the airway or vessel wall, respectively (Finkelstein et al., 1995; Finkelstei et al., 1995a; Hasson et al., 2005). Of note, smokers with airflow limitation have more prominent atherosclerosis than smokers with normal lung function, suggesting a link between atherosclerosis and obstructive lung disease (Iwamoto et al., 2009). Atherosclerotic lesions exhibit increased numbers of lipid-laden macrophages (Hasson et al., 2005). In addition the accumulation of foamy alveolar macrophages was also observed in smoke-exposed mice (Hirama et al., 2007). Interestingly, passive smoking is known to influence plasma lipid concentrations (de Padua Mansur et al., 1997; Moskowitz et al., 1990; Feldman et al., 1991), most significantly decreasing HDL levels (Moffatt et al., 2004). Cigarette smoke is also associated with increased levels of oxidized low-density lipoprotein (LDL) cholesterol, which damages the vessel endothelium (Stokes et al., 1990). In addition, pulmonary emphysema has been associated with increased levels of sphingolipids (Petrache et al., 2005), particularly ceramides and sphingomyelin. Furthermore exogenous administration of ceramide to the lung correlated with emphysema formation (Petrache et al., 2005) while increasing levels of its by product sphingosine-1-phosphate (S1P) blocked emphysema formation (Diab et al., 2010; Yasuo et al., 2013). Despite the importance of these lipids in the vascular wall integrity and emphysema pathogenesis, the consequences of lung lipid changes and their effect on cigarette smoke induced inflammation and lung destruction have not been fully examined.

Discussion

Emphysema is a form of chronic obstructive pulmonary disease (COPD) with no known cure. It occurs when the linings of the air sacs in the lungs become irreversibly damaged, limiting the ability for patients with this disease to breathe. The most common cause of emphysema is cigarette smoking. Smoking leads to an increase in the expression of enzymes called matrix metalloproteinases (MMPs), which are primarily responsible for the lung damage. The technology disclosed herein offers new insight and a therapeutic pathway for treating emphysema. It does so by identifying the beneficial effects of Liver X receptors (LXR) agonists, which impact cholesterol efflux capacity. The technology herein shows In vitro data that LXR agonists can reduce smoke-induced MMPs. In addition, mouse model in vivo data demonstrates that LXR agonist treatment can repair acute cigarette induced smoke pulmonary inflammation. Currently, LXR agonists are used as a treatment in mouse models of Alzheimer's disease, atherosclerosis, diabetes, and anti-inflammation. Aspects of the present invention relate to the identification of LXR agonists useful for treating patients suffering from emphysema, including but not limited to chronic cigarette smoke induced emphysema.

Matrix metalloproteinases (MMPs) are key enzymes responsible for the lung destruction seen in emphysema. It is also well established that cigarette smoke leads to the infiltration of macrophages and induces the expression of MMPs through the TLR signaling pathway. Recent studies conducted by the inventors demonstrate that abnormalities in cholesterol efflux and cholesterol homeostasis also contribute to emphysema development in ApoE KO mice. These data demonstrate that cigarette smoke exposure leads to impaired cholesterol efflux and down-regulation of the cholesterol transporter ABCA1 in bone marrow derived macrophages also in a TLR4 signaling dependent manner.

Importantly, the data herein demonstrate that LXR agonists, which increase ABCA1 (main LXR target gene) expression and its dependent cholesterol efflux capacity, can reduce smoke-induced destructive MMPs and proinflammatory cytokines such as TNFα. The LXR agonist(T0901317) induced decrease in MMP-9 activity (assessed by zymography) and expression, MMP-13 expression and TNFα expression in macrophages was ABCA1-dependent since the effect was not observed in ABCA1 KO macrophages.

In order to determine if the mechanism identified in vitro contributed to changes in smoke induced lung inflammation in vivo, smoke studies were performed in vivo. Short term (10 days) cigarette smoke exposure of mice was performed and treatment with 25 mg/kg (Intraperitonal injection—start after 5 days of pulmonary inflammation establishment for 5 consecutive days) was observed to resolve pulmonary inflammation measured by total inflammatory cell count in bronchioalveolar fluid after performing bronchioalveolar lavage (BAL). Without wishing to be bound by any scientific theory, this effect was potentially also ABCA1-dependent as macrophage ABCA1-deficient mice exposed to cigarette smoke for 10 days had significantly more inflammation in the lung (measured by total cell counts in BAL) and the treatment of these macrophage-ABCA1-deficient mice did not resolve the inflammation as it did in wild type mice suggesting importance of ABCA1 in LXR driven resolution of cigarette smoke induced pulmonary inflammation. Recent studies have demonstrated that hypercholesterolemia contributes to emphysema development in ApoE KO mice (Goldklang, M., D'Armiento, Am J Physiol Lung Cell Mol Physiol. 2012 Jun. 1; 302(11)). The data presented herein suggests abnormal cholesterol efflux driven inflammatory signaling and destructive MMP induction as one of crucial mechanism in emphysema development. Through investigation of the smoke-induced regulation of ABCA1, the present disclosure identifies unique pathways that can be targeted to prevent smoking-related emphysema and the progression of lung destruction.

Without wishing to be bound by any scientific theory, based on the above data, smoke regulation of cholesterol transport, inflammation and lung tissue destruction by MMPs all appear to be mechanistically linked, playing a role in the increased risk of emphysema development in smokers and potentially presenting a therapeutic target. Thus, the present disclosure fills a critical gap in the existing literature and provides insight into how tobacco smoke induced impaired cholesterol efflux affects emphysema development. As shown herein, LXR agonists are useful for the treatment of chronic cigarette smoke induced emphysema in mice (ABCA1 deficient in macrophages).

LXR agonists are effective for treatment of murine models of atherosclerosis, diabetes, anti-inflammation, and Alzheimer's disease. Treatment with LXR agonists (hypocholamide, T0901317, GW3965, or N,N-dimethyl-3beta-hydroxy-cholenamide (DMHCA)) lowers the cholesterol level in serum and liver and inhibits the development of atherosclerosis in murine disease models.

A recent study has reported that GW3965 did not significantly suppress the production of TNFα, IL-l)3, or CXCL8, but had anti-inflammatory effects on CXC10, CCL5, and IL-10 production in alveolar macrophages in vitro. Hingham et al (2013) “The role of the liver X receptor in chronic obstructive pulmonary disease” Respiratory Research 14:106. However, the same study ruled out “ . . . a potentially therapeutic role for LXR agonists in altering macrophage phenotype in COPD.” Additionally, Hingham et al (2013) taught that “[t]he restricted nature of the anti-inflammatory activity of LXR on selected cytokines in lymphocytes, coupled with the reduced effect size compared to corticosteroids, makes it unlikely that the in-vitro anti-inflammatory effects reported here would translate into clinically meaningful benefits in COPD patients.” The discovery herein that LXR agonists are useful for treatment of COPD in subjects is surprising because it contradicts this report in the art.

The present invention provides novel pharmacotherapies for COPD comprising LXR agonists, miR-33 antagonists, and/or TLR4/Myd8,8 pathway antagonists.

### REFERENCES

Podowski M, Calvi C, Metzger S, Misono K, Poonyagariyagorn H, Lopez-Mercado A, et al. Angiotensin receptor blockade attenuates cigarette smoke-induced lung injury and rescues lung architecture in mice. J Clin Invest. 2012; 122(1) :229-40. PubMed Central PMCID: PMC3248282.
Mannino D M, Buist A S. Global burden of COPD: risk factors, prevalence, and future trends. Lancet. 2007; 370(9589) :765-73.
Global strategy for the diagnosis, management, and prevention of chronic obstructive pulmonary disease. Global Initiative for Chronic Obstructive Lung Disease, 2011.
Global strategy for the diagnosis, management, and prevention of chronic obstructive pulmonary disease. Global Initiative for chronic obstructive Lung disease., 2007.
Yoshida T, Tuder R M. Pathobiology of cigarette smoke-induced chronic obstructive pulmonary disease. Physiol Rev. 2007; 87(3) :1047-82.
Celli B R. Roger s. Mitchell lecture. Chronic obstructive pulmonary disease phenotypes and their clinical relevance. Proc Am Thorac Soc. 2006; 3(6) :461-5.

Shan M, Cheng H F, Song L Z, Roberts L, Green L, Hacken-Bitar J, et al. Lung myeloid dendritic cells coordinately induce TH1 and TR17 responses in human emphysema. Sri Transl Med. 20091(4) :4ra10.
Shaykhiev R, Krause A, Salit J, Strulovici-Barel Y, Harvey BG, O'Connor T P, et al. Smoking-dependent reprogramming of alveolar macrophage polarization: implication for pathogenesis of chronic obstructive pulmonary disease. J Immunol. 2009; 183(4) :2867-83. PubMed Central PMCID: PMC2873685.
Finkelstein R, Fraser R S, Ghezzo H, Casio M G. Alveolar inflammation and its relation to emphysema in smokers. Am J Respir Crit Care Med. 1995; 152(5 Pt 1) :1666-72.
Shapiro S D. The macrophage in chronic obstructive pulmonary disease. Am J Respir Crit Care Med. 1999; 160(5 Pt 2) :S29-32.
Pons A R, Sauleda J, Noguera A, Pons J, Barcelo B, Fuster A, et al. Decreased macrophage release of TGF-beta and TIMP-1 in chronic obstructive pulmonary disease. Eur Respir J. 2005; 26(1) :60-6.
Finlay G A, Russell K J, McMahon K J, D'Arcy E M, Masterson J B, FitzGerald M X, et al. Elevated levels of matrix metalloproteinases in bronchoalveolar lavage fluid of emphysematous patients. Thorax. 1997; 52(6) :502-6. PubMed Central PMCID: PMC1758576.
Finlay G A, O'Driscoll L R, Russell K J, D'Arcy E M, Masterson J B, FitzGerald M X, et al. Matrix metalloproteinase expression and production by alveolar macrophages in emphysema. Am J Respir Crit Care Med. 1997a; 156(1) :240-7.
Woodruff P G, Koth L L, Yang Y H, Rodriguez M W, Favoreto S, Dolganov G M, et al. A distinctive alveolar macrophage activation state induced by cigarette smoking. Am J Respir Crit Care Med. 2005; 172(11) :1383-92. PubMed Central PMCID: PMC2718436.
Foronjy R, Nkyimbeng T, Wallace A, Thankachen J, Okada Y, Lemaitre V, et al. Transgenic expression of matrix metalloproteinase-9 causes adult-onset emphysema in mice associated with the loss of alveolar elastin. Am J Physiol Lung Cell Mol Physiol. 2008; 294(6) : L1149-57.
D'Armiento J, Dalal S S, Okada Y, Berg R A, Chada K. Collagenase expression in the lungs of transgenic mice causes pulmonary emphysema. Cell. 1992; 71(6) :955-61.
Hautamaki R D, Kobayashi D K, Senior R M, Shapiro S D. Requirement for macrophage elastase for cigarette smoke-induced emphysema in mice. Science. 1997; 277(5334) :2002-4.
Thomson E M, Williams A, Yauk C L, Vincent R. Overexpression of tumor necrosis factoralpha in the lungs alters immune response, matrix remodeling, and repair and maintenance pathways. Am J Pathol. 2012; 180(4) :1413-30.
Letuve S, Kozhich A, Arouche N, Grandsaigne M, Reed J, Dombret M C, et al. YKL-40 is elevated in patients with chronic obstructive pulmonary disease and activates alveolar macrophages. J Immunol. 2008; 181(7) :5167-73.
Churg A, Zhou S, Wang X, Wang R, Wright J L. The role of interleukin-1beta in murine cigarette smoke-induced emphysema and small airway remodeling. Am J Respir Cell Mol Biol. 2009; 40(4) :482-90.
Couillin I, Vasseur V, Charron S, Gasse P, Tavernier M, Guillet J, et al. IL-1R1/MyD88 signaling is critical for elastase-induced lung inflammation and emphysema. J Immunol. 2009; 183(12) :8195-202.
Doz E, Noulin N, Boichot E, Guenon I, Fick L, Le Bert M, et al. Cigarette smoke-induced pulmonary inflammation is TLR4/MyD88 and IL-1R1/MyD88 signaling dependent. J Immunol. 2008; 180(2) :1169-78.
Karimi K, Sarir H, Mortaz E, Smit J J, Hosseini H, De Kimpe S J, et al. Toll-like receptor-4 mediates cigarette smoke-induced cytokine production by human macrophages. Respir Res. 2006; 7:66. PubMed Central PMCID: PMC1481582.
Zheng T, Zhu Z, Wang Z, Homer R J, Ma B, Riese R J, Jr., et al. Inducible targeting of IL-13 to the adult lung causes matrix metalloproteinase- and cathepsin-dependent emphysema. J Clin Invest. 2000; 106(9) :1081-93. PubMed Central PMCID: PMC301418.
Wang Z, Zheng T, Zhu Z, Homer R J, Riese R J, Chapman H A, Jr., et al. Interferon gamma induction of pulmonary emphysema in the adult murine lung. J Exp Med. 2000; 192(11) :1587-600. PubMed Central PMCID: PMC2193095.
Geraghty P, Dab A J, D'Armiento J. TLR4 protein contributes to cigarette smoke-induced matrix metalloproteinase-1 (MMP-1) expression in chronic obstructive pulmonary disease, J Bioi Chem. 2011; 286(34) :30211-8. PubMed Central PMCID: PMC3191060.
Holland W L, Summers S A. Sphingolipids, insulin resistance, and metabolic disease: new insights from in vivo manipulation of sphingolipid metabolism. Endocrine reviews. 2008; 29(4) :381-402. PubMed Central PMCID: PMC2528849.
Petrache I, Natarajan V, Zhen L, Medler T R, Richter A T, Cho C, et al. Ceramide upregulation causes pulmonary cell apoptosis and emphysema-like disease in mice. Nat Med. 2005; 11(5) :491-8. PubMed Central PMCID: PMC1352344.
Barnoya J, Glantz S A. Cardiovascular effects of secondhand smoke: nearly as large as smoking. Circulation. 2005; 111(20) :2684-98.
Freund K M, Belanger A J, D'Agostino R B, Kannel W B. The health risks of smoking. The Framingham Study: 34 years of follow-up. Ann Epidemiol. 1993; 3(4) :417-24.
Howard G, Wagenknecht L E, Burke G L, Diez-Roux A, Evans G W, McGovern P, et al. Cigarette smoking and progression of atherosclerosis: The Atherosclerosis Risk in Communities (ARIC) Study. JAMA. 1998; 279(2) :119-24.
Milei J, Grana DR. Mortality and morbidity from smoking-induced cardiovascular diseases: the necessity of the cardiologist's involvement and commitment. Int J Cardiol. 1998; 67(2) :95-109.
Finkelstein R, Ma H D, Ghezzo H, Whittaker K, Fraser R S, Cosio M G. Morphometry of small airways in smokers and its relationship to emphysema type and hyperresponsiveness. Am J Respir Crit Care Med. 1995a; 152(1) :267-76.
Hansson G K. Inflammation, atherosclerosis, and coronary artery disease. N Engl J Med. 2005; 352(16) :1685-95.
Iwamoto H, Yokoyama A, Kitahara Y, Ishikawa N, Haruta Y, Yamane K, et al. Airflow limitation in smokers is associated with subclinical atherosclerosis. Am J Respir Crit Care Med. 2009; 179(1) :35-40.
Hirama N, Shibata Y, Otake K, Machiya J, Wada T, Inoue S, et al. Increased surfactant protein-D and foamy macrophages in smoking-induced mouse emphysema. Respirology. 2007; 12(2) :191-201.
de Padua Mansur A, Caramelli B, Vianna C B, Chamone D, Ramires J A. Smoking and lipoprotein abnormalities on platelet aggremation in coronary heart disease. Int J Cardiol. 1997; 62(2) :151-4.
Moskowitz W B, Mosteller M, Schieken R M, Bossano R, Hewitt J K, Bodurtha J N, et al. Lipoprotein and oxygen transport alterations in passive smoking preadolescent children. The MCV Twin Study. Circulation. 1990; 81(2) :586-92.
Feldman J, Shenker I R, Etzel R A, Spierto F W, Lilienfield D E, Nussbaum M, et al. Passive smoking alters lipid profiles in adolescents. Pediatrics. 1991; 88(2) :259-64.
Moffatt R J, Chelland S A, Pecott D L, Stamford B A. Acute exposure to environmental tobacco smoke reduces HDL-C and HDL2-C. Prev Med. 2004; 38(5) :637-41.
Stokes J, 3rd. Cardiovascular risk factors. Cardiovasc Clin. 1990; 20(3) :3-20.
Diab K J, Adamowicz J J, Kamocki K, Rush N I, Garrison J, Gu Y, et al. Stimulation of sphingosine 1-phosphate signaling as an alveolar cell survival strategy in emphysema. Am J Respir Crit Care Med. 2010; 181(4) :344-52.
Yasuo M, Mizuno S, Allegood J, Kraskauskas D, Bogaard H J, Spiegel S, et al. Fenretinide causes emphysema, which is prevented by sphingosine 1-phoshate. PLoS One. 2013; 8(1) :e53927. PubMed Central PMCID: PMC3543313.
Goldklang M, Golovatch P, Zelonina T, Trischler J, Rabinowitz D, Lemaitre V, et al. Activation of the TLR4 signaling pathway and abnormal cholesterol efflux lead to emphysema in ApoEdeficient mice. Am J Physiol Lung Cell Mol Physiol. 2012; 302(11) :L1200-8. PubMed Central PMCID:PMC3379042.
Tall A R, Costet P, Wang N. Regulation and mechanisms of macrophage cholesterol efflux. J Clin Invest. 2002; 110(7) :899-904. PubMed Central PMCID: PMC151157.
Ranalletta M, Wang N, Han S, Yvan-Charvet L, Welch C, Tall A R. Decreased atherosclerosis in low-density lipoprotein receptor knockout mice transplanted with Abcg1−/− bone marrow. Arterioscler Thromb Vasc Biol. 2006; 26(10) :2308-15.
Tall A R, YeanaCharvet L, Terasaka N, Nagler T, Wang N. HDL, ABC transporters, and cholesterol efflux: implications for the treatment of atherosclerosis, Cell Metab, 2008; 7(5) :365-75.
Wang N, Lan D, Chen W, Matsuura F, Tall A R. ATP-binding cassette transporters G1 and G4 mediate cellular cholesterol efflux to high-density lipoproteins. Proc Natl Acad Sci USA. 2004; 101(26) :9774-9. PubMed Central PMCID: PMCPMC470750.
Yvan-Charvet L, Pagler T A, Seimon T A, Thorp E, Welch C L, Witztum J L, et al. ABCA1 and ABCG1 protect against oxidative stress-induced macrophage apoptosis during efferocytosis. Circ Res.106(12) :1861-9. PubMed Central PMCID: PMC2995809.
Yvan-Charvet L, Wang N, Tall A R. Role of HDL, ABCA1, and ABCG1 transporters in cholesterol efflux and immune responses. Arterioscler Thromb Vasc Biol. 2010a 30(2) :139-43. PubMed Central PMCID: PMC2812788.
Yvan-Charvet L, Ranalletta M, Wang N, Han S, Terasaka N, Li R, et al. Combined deficiency of ABCA1 and ABCG1 promotes foam cell accumulation and accelerates atherosclerosis in mice. J Clin Invest. 2007; 117(12) :3900-8. PubMed Central PMCID: PMCPMC2066200.
Baldan A, Gomes A V, Ping P, Edwards P A. Loss of ABCG1 results in chronic pulmonary inflammation. J Immunol. 2008; 180(5) :3560-8.
Bates S R, Tao J Q, Collins H L, Francone O L, Rothblat G H. Pulmonary abnormalities due to ABCA1 deficiency in mice. Am J Physiol Lung Cell Mol Physiol. 2005; 289(6) :L980-9.
Yvan-Charvet L, Welch C, Pagler T A, Ranalletta M, Lamkanfi M, Han S, et al. Increased inflammatory gene expression in ABC transporter-deficient macrophages: free cholesterol accumulation, increased signaling via toll-like receptors, and neutrophil infiltration of atherosclerotic lesions. Circulation. 2008; 118(18) :1837-47. PubMed Central PMCID: PMCPMC2756536.
Adorni M P, Zimetti F, Billheimer J T, Wang N, Rader D J, Phillips M C, et al. The roles of different pathways in the release of cholesterol from macrophages. J Lipid Res. 2007; 48(11) :2453-62.
Out R, Jessup W, Le Goff W, Hoekstra M, Gelissen I C, Zhao Y, et al. coexistence of foam cells and hypocholesterolemia in mice lacking the ABC transporters A1 and C1. Circ Res. 2008; 102(1) :113-20.
Beaven S W, Tontonoz P. Nuclear receptors in lipid metabolism: targeting the heart of dyslipidemia. Annu Rev Med. 2006; 57:313-29.
Larrede S, Quinn C M, Jessup W, Frisdal E, Olivier M, Hsieh V, et al. Stimulation of cholesterol efflux by LXR agonists in cholesterol-loaded human macrophages is ABCA1-dependent but ABCG1-independent. Arterioscler Thromb Vasc Biol. 2009; 29(11) :1930-6.
Rayner K J, Suarez Y, Davalos A, Parathath S, Fitzgerald M L, Tamehiro N, et al. MiR-33 contributes to the regulation of cholesterol homeostasis. Science. 2010, 328(5985) :1570-3. PubMed Central PMCID: PMCPMC3114628.
Castrillo A, Joseph S B, Vaidya S A, Haberland M, Fogelman A M, Cheng G, et al. Crosstalk between LXR and toll-like receptor signaling mediates bacterial and viral antagonism of cholesterol metabolism. Mol Cell. 2003; 12(4) :805-16.
Zhu X, Lee J Y, Timmins J M, Brown J M, Boudyguina E, Mulya A, et al. Increased cellular free cholesterol in macrophage-specific Abcal knock-out mice enhances pro-inflammatory response of macrophages. J Biol Chem. 2008; 283(34) :22930-41. PubMed Central PMCID: PMCPMC2516976.
Zhu X, Owen J S, Wilson M D, Li H, Griffiths G L, Thomas M J, et al. Macrophage ABCA1 reduces MyD88-dependent Toll-like receptor trafficking to lipid rafts by reduction of lipid raft cholesterol. J Lipid Res. 2010; 51 :3196-206. PubMed Central PMCID: PMC2952560.
Zhou J, Eksioglu E A, Fortenbery N R, Chen X, Wang H, Epling-Burnette PK, et al. Bone marrow mononuclear cells up-regulate toll-like receptor expression and produce inflammatory mediators in response to cigarette smoke extract. PLoS One.6(6) :e21173. 2010 PubMed Central PMCID: PMCPMC3126797.
Petrusca D N, Gu Y, Adamowicz J J, Rush N I, Hubbard W C, Smith P A, et al. Sphingolipidmediated inhibition of apoptotic cell clearance by alveolar macrophages. J Biol Chem. 2010; 285(51) :40122-32. PubMed Central PMCID: DMC3001012
Dehle F C, Mukaro V R, Jurisevic C, Moffat D, Ahern J, Hodge G, et al. Defective lung macrophage function in lung Cancer +/− chronic obstructive pulmonary disease (COPD/Emphysema)—mediated by cancer cell production of PGE2? PLoS One. 2013; 8(4) :e61573. PubMed Central PMCID: PMC3637201.
Foronjy R F, Mercer B A, Maxfield M W, Powell C A, D'Armiento J, Okada Y. Structural emphysema does not correlate with lung compliance: lessons from the mouse smoking model. Exp Lung Res. 2005; 31(6) :547-62.

Foronjy R F, Mirochnitchenko O, Propokenko O, Lemaitre V, Jia Y, Inouye M, et al. Superoxide dismutase expression attenuates cigarette smoke- or elastase-generated emphysema in mice. Am J Respir Crit Care Med. 2006; 173(6) :623-31.
Foronjy R F, Okada Y, Cole R, D'Armiento J. Progressive adult-onset emphysema in transgenic mice expressing human MMP-1 in the lung. Am J Physiol Lung Cell Mol Physiol. 2003; 284(5) :L727-37.
Geraghty P, Dabo A J, D'Armiento J. TLR4 Protein Contributes to Cigarette Smoke induced Matrix Metalloproteinase-1 (MMP-1) Expression in Chronic Obstructive Pulmonary Disease. J Biol Chem. 2011 286(34) :30211-8.
Golovatch P, Mercer B A, Lemaitre V, Wallace A, Foronjy R F, D'Armiento J. Role for cathepsin K in emphysema in smoke-exposed guinea pigs. Exp Lung Res. 2009; 35(8) :631-45. PubMed Central PMCID: PMCPMC2980838.
Thurlbeck W M, Dunnill M S, Hartung W, Heard B E, Heppleston A G, Ryder R C. A comparison of three methods of measuring emphysema. Hum Pathol. 1970; 1(2) :215-26.
Woessner J F, Jr. The determination of hydroxyproline in tissue and protein samples containing small proportions of this imino acid. Arch Biochem Biophys. 1961; 93:440-7.
Libby P. Inflammation in atherosclerosis. Nature. 2002; 420(6917) :868-74.
Plump A S, Scott C J, Breslow J L. Human apolipoprotein A-I gene expression increases high density lipoprotein and suppresses atherosclerosis in the apolipoprotein E-deficient mouse. Proc Natl Acad Sci USA. 1994; 91(20) :9607-11. PubMed Central PMCID: PMCPMC44862.
Im S S, Osborne T F. Liver x receptors in atherosclerosis and inflammation. Circ Res. 2011108(8) :996-1001. PubMed Central PMCID: PMC3082200.
Joseph S B, McKilligin E, Pei L, Watson M A, Collins A R, Laffitte B A, et al. Synthetic LXR ligand inhibits the development of atherosclerosis in mice. Proc Natl Acad Sci USA. 2002; 99(11) :7604-9. PubMed Central PMCID: PMC124297. doi:10.1073/pnas.112059299. PMID 12032330.
Levin N, Bischoff E D, Daige C L, Thomas D, Vu CT, Heyman R A, et al. Macrophage liver X receptor is required for antiatherogenic activity of LXR agonists. Arterioscler Thromb Vasc Biol. 2005; 25(1) :135-42.
Wang N, Ranalletta M, Matsuura F, Peng F, Tall AR. LXR-induced redistribution of ABCG1 to plasma membrane in macrophages enhances cholesterol mass efflux to HDL. Arterioscler Thromb Vasc Biol. 2006; 26(6) :1310-6.
Rayner K J, Sheedy F J, Esau C C, Hussain F N, Temel R E, Parathath S, et al. Antagonism of miR-33 in mice promotes reverse cholesterol transport and regression of atherosclerosis. J Clin Invest. 121(7) :2921-31.
Marcos P A, Li Y, Jiang S. Vivo-Morpholinos: a non-peptide transporter delivers Morpholinos into a wide array of mouse tissues. Biotechniques. 2008; 45(6) :613-4, 6, 8 passim.
Moulton J D, Jiang S. Gene knockdowns in adult animals: PPMOs and vivo-morpholinos. Molecules. 2009; 14(3) :1304-23.
Liu X, Xiang S L, Yi G H. ABCA1, ABCG1, and SR-BI: Transit of HDL-associated sphingosine-1-phosphate. Clinica chimica acta; international journal of clinical chemistry. 2012; 413(3-4) :384-90.
Okamoto Y, Yoshioka K, Takuwa N, Takuwa Y. [Cardiovascular function of sphingosine 1-phosphate signaling]. Seikagaku The Journal of Japanese Biochemical Society. 2011; 83(6) :536-44.
Sano O, Kobayashi A, Nagao K, Kumagai K, Kioka N, Hanada K, et al. Sphingomyelindependence of cholesterol efflux mediated by ABCG1. J Lipid Res. 2007; 48(11) :2377-84.
Kobayashi A, Takanezawa Y, Hirata T, Shimizu Y, Misasa K, Kioka N, et al. Efflux of sphingomyelin, cholesterol, and phosphatidylcholine by ABCG1. J Lipid Res. 2006; 47(8) :1791-802.
Ghering A B, Davidson W S. Ceramide structural features required to stimulate ABCA1-mediated cholesterol efflux to apolipoprotein A-I. J Lipid Res. 2006; 47(12) :2781-8.
Sato K, Malchinkhuu E, Horiuchi Y, Mogi C, Tomura H, Tosaka M, et al. Critical role of ABCA1 transporter in sphingosine 1-phosphate release from astrocytes. Journal of neurochemistry. 2007; 103(6) :2610-9.
Fletcher J I, Haber M, Henderson M J, Norris M D. ABC transporters in cancer: more than just drug efflux pumps. Nature reviews Cancer. 2010; 10(2) :147-56.
Yang Y, Uhlig S. The role of sphingolipids in respiratory disease. Therapeutic advances in respiratory disease. 2011; 5(5) :325-44.
Worgall T S. Sphingolipid synthetic pathways are major regulators of lipid homeostasis. Advances in experimental medicine and biology. 2011; 721:139-48.
Clugston R D, Jiang H, Lee M X, Piantedosi R, Yuen J J, Ramakrishnan R, et al. Altered hepatic lipid metabolism in C57BL/6 mice fed alcohol: a targeted lipidomic and gene expression study. J Lipid Res. 2011; 52(11) :2021-31. PubMed Central PMCID: PMC3196234.
Imai K, Mercer B A, Schulman L L, Sonett JR, D'Armiento J M. Correlation of lung surface area to apoptosis and proliferation in human emphysema. The European respiratory journal: official journal of the European Society for Clinical Respiratory Physiology. 2005; 25(2) :250-8.
Roth M D, Connett J E, D'Armiento J M, Foronjy R F, Friedman P J, Goldin J G, et al. Feasibility of retinoids for the treatment of emphysema study. Chest. 2006; 130(5) :1334-45.
Viennois, E et al. Targeting liver X receptors in human health: deadlock or promising trail? Expert Opin Ther Targets. 2011 February; 15(2) :219-32.
Center for Disease Control and Prevention. FastStats—Chronic Lower Respiratory Disease, Jan. 11, 2013
Webster, N. & and Crowe, S. Matrix metalloproteinases, their production by monocytes and macrophages and their potential role in HIV-related diseases. 2006 Leukoc. Biol. 80, 1052-1066.
Alberti S, Schuster G, Parini P, Feltkamp D, Diczfalusy U, Rudling M, Angelin B, Björkhem I, Pettersson S, Gustafsson J A (March 2001). “Hepatic cholesterol metabolism and resistance to dietary cholesterol in LXRβ-deficient mice”. J. Clin. Invest. 107 (5) : 565-73. doi:10.1172/JCI9794. PMC 199420. PMID 11238557.
Song C, Hiipakka R A, Liao S (June 2001). “Auto-oxidized cholesterol sulfates are antagonistic ligands of liver X receptors: implications for the development and treatment of atherosclerosis”. Steroids 66 (6) : 473-9. doi:10.1016/S0039-128X(00)00239-7. PMID 11182136.
Kratzer A, Buchebner M, Pfeifer T, Becker T M, Uray G, Miyazaki M, Miyazaki-Anzai S, Ebner B, Chandak P G, Kadam R S, Calayir E, Rathke N, Ahammer H, Radovic B, Trauner M, Hoefler G, Kompella U B, Fauler G, Levi M, Levak-Frank S, Kostner G M, Kratky D (February 2009). “Synthetic LXR agonist attenuates plaque formation in apoE−/− mice without inducing liver steatosis and hypertriglyceridemia”. J. Lipid Res. 50 (2) : 312-26. doi:10.1194/jlr.M800376-JLR200.
Barnoya J, and Glantz S A. Cardiovascular effects of secondhand smoke: nearly as large as smoking. Circulation. 2005; 111(20) :2684-98.
Cirillo D J, Agrawal Y, and Cassano P A. Lipids and pulmonary function in the Third National Health and Nutrition Examination Survey. Am J Epidemiol. 2002; 155(9) :842-8.
Tall A R, Costet P, and Wang N. Regulation and mechanisms of macrophage cholesterol efflux. The Journal of clinical investigation. 2002; 110(7) :899-904.
Yvan-Charvet L, Pagler T A, Simon T A, Thorp E, Welch C L, Witztum J L, Tabas I, and Tall A R. ABCA1 and ABCG1 protect against oxidative stress-induced macrophage apoptosis during efferocytosis. Circ Res.2010a, 106(12) :1861-9.
Buist A S, McBurnie M A, Vollmer W M, Gillespie S, Burney P, Mannino D M, Menezes A M, Sullivan S D, Lee T A, Weiss K B, et al. International variation in the prevalence of COPD (the BOLD Study) : a population-based prevalence study. Lancet. 2007; 370(9589):741-50.
Foronjy R, and D'Armiento J. The role of collagenase in emphysema. Respir Res. 2001:2(6) :348-52
Foronjy R, Imai K, Shiomi T, Mercer B, Sklepkiewicz P, Thankachen J, Bodine P, and D'Armiento J. The divergent roles of secreted frizzled related protein-1 (SFRP1) in lung morphogenesis and emphysema. Am J Pathol. 2010, 177(2) :598-607.
Foronjy R, Wallace A, and D'Armiento J. The pharmokinetic limitations of antioxidant treatment for COPD. Pulm Pharmacol Ther. 2008a; 21(2) :370-9.
Geraghty P, Hardigan A A, Wallace A M, Mirochnitchenko O, Thankachen J, Arellanos L, Thompson V, D'Armiento J M, and Foronjy R R. The GPx1-PTP1B-PP2A Axis: A Key Determinant of Airway Inflammation and Alveolar Destruction. Am J Respir Cell Mol Biol. 2013.
Mercer B A, Kolesnikova N, Sonett J, and D'Armiento J. Extracellular regulated kinase/mitogen activated protein kinase is up-regulated in pulmonary emphysema and mediates matrix metalloproteinase-1 induction by cigarette smoke. J Biol Chem. 2004; 279(17) :17690-6.
Petrache I, Kamocki K, Poirier C, Pewzner-Jung Y, Laviad E L, Schweitzer K S, Van Demark M, Justice M J, Hubbard W C, and Futerman A H. Ceramide synthases expression and role of ceramide synthase-2 in the lung: insight from human lung cells and mouse models. PLoS One. 2013; 8(5) :e62968.
Petrache I, and Petrusca D N. The involvement of sphingolipids in chronic obstructive pulmonary diseases. Handbook of experimental pharmacology. 2013; 216(247-64.

What is claimed is:

1. A method for treating a subject afflicted with chronic obstructive pulmonary disease (COPD) which comprises administering to the subject i) a Liver X receptor (LXR) agonist, ii) a miR-33 antagonist, or iii) a TLR4/Myd88 pathway antagonist in an amount that is effective to treat the subject.

2. The method of claim 1, wherein treating the subject comprises improving pulmonary function in the subject or reducing pulmonary inflammation in the subject.

3. The method of claim 1 or 2, wherein the pulmonary inflammation is reduced by about 10, 20, 30, 40, 50, 60, 70, 80, 90, or 100% in the subject compared to the level of pulmonary inflammation when the subject was first administered the LXR agonist, the miR-33 antagonist, or the TLR4/Myd88 pathway antagonist.

4. The method of any one of claims 1-3, wherein the COPD comprises emphysema.

5. The method of any one of claims 1-4, wherein treating the subject comprises
a) reducing emphysema in the subject;
b) slowing or halting the progression of emphysema in the subject;
c) reversing emphysema in the subject;
d) reversing emphysema in the subject, wherein the emphysema is reversed by about 10, 20, 30, 40, 50, 60, 70, 80, 90, or 100% in the subject compared to the level of emphysema when the subject was first administered the LXR agonist, the miR-33 antagonist, or the TLR4/Myd88 pathway antagonist;
e) reducing obstructive bronchiolitis in the subject;
f) reducing mucus hypersecretion in the subject;
g) reducing pulmonary compliance in the subject;
h) reducing alveolar or bronchial infiltration of at least one type of inflammatory cell in the subject;
i) reducing alveolar or bronchial infiltration of at least one type of inflammatory cell in the subject, wherein the at least one type of inflammatory cell comprises macrophages or foamy macrophages;
j) reducing alveolar or bronchial infiltration of at least one type of inflammatory cell in the subject, wherein the alveolar or bronchial infiltration is reduced by about 10, 20, 30, 40, 50, 60, 70, 80, 90, or 100% in the subject compared to the level of alveolar or bronchial infiltration when the subject was first administered the LXR agonist, the miR-33 antagonist, or the TLR4/Myd88 pathway antagonist;
k) reducing pulmonary compliance in the subject, wherein pulmonary compliance is reduced by about 1, 2, 3, 4, 5, 6, 7, 8, 9, 10, 15, 20 or 25% in the subject compared to the level of pulmonary compliance when the subject was first administered the LXR agonist, the miR-33 antagonist, or the TLR4/Myd88 pathway antagonist;
l) reducing pulmonary compliance in the subject, wherein the pulmonary compliance is static pulmonary compliance or dynamic compliance;
m) increasing pulmonary elastance in the subject;
n) increasing pulmonary elastance in the subject, wherein pulmonary elastance is increased by about 1, 2, 3, 4, 5, 6, 7, 8, 9, 10, 15, 20 or 25% in the subject compared to the level of pulmonary elastance when the subject was first administered the LXR agonist, the miR-33 antagonist, or the TLR4/Myd88 pathway antagonist;
o) increasing pulmonary resistance in the subject; or
p) increasing pulmonary resistance in the subject, wherein pulmonary resistance is increased by about 1, 2, 3, 4, 5, 6, 7, 8, 9, 10, 15, 20 or 25% in the subject compared to the level of pulmonary resistance when the subject was first administered the LXR agonist, the miR-33 antagonist, or the TLR4/Myd88 pathway antagonist.

6. The method of any one of claims 1-5, wherein the LXR agonist, the miR-33 antagonist, or the TLR4/Myd88 pathway antagonist
a) decreases the expression or enzymatic activity of at least one matrix metalloproteinase (MMP), in the subject;
b) decreases the expression of at least one cytokine, in the subject;
c) decreases the level of a ceramide in the lungs or the serum of the subject;
d) decreases the level of a ceramide in the lungs or the serum of the subject, wherein the ceramide is a C14 or a C16 ceramide;
e) increases ABCA1 or ABCG1 expression in the subject;
f) increases the level of sphingosine 1-phosphate (S1P) in the lungs or the serum of the subject; or
g) increases LXR expression in the subject.

7. The method of any one of claims 1-6, wherein a LXR agonist is administered to the subject.

8. The method of claim 7, wherein the LXR agonise is
a) an LXRα agonist;
b) an LXRβ agonist;
c) an LXRα and LXRβ agonist; or
d) also a farnesoid X receptor (FXR) agonist.

9. The method of claim 7 or 8, wherein the LXR agonist is an organic compound having a molecular weight less than 1000 Daltons, a DNA aptamer, an RNA aptamer, or a polypeptide.

10. The method of claim 9, wherein the LXR agonist is
a) T0901317 or a pharmaceutically acceptable salt or ester thereof;
b) GW3965 or a pharmaceutically acceptable salt or ester thereof;
c) EXEL2255 or a pharmaceutically acceptable salt or ester thereof;
d) N,N-dimethyl-3β-hydroxy-cholenamide (DMHCA) or a pharmaceutically acceptable salt or ester thereof;
e) BMS-779788 or a pharmaceutically acceptable salt or ester thereof;
f) an sLXRM or a pharmaceutically acceptable salt or ester thereof;
g) other than GW3965 or a pharmaceutically acceptable salt or ester thereof; or
h) in a clinical trial or is approved for use in treating atherosclerosis.

11. The method of any one of claims 1-10, wherein the amount of the LXR agonist administered is less than the amount that is effective for treatment of atherosclerosis.

12. The method of any one of claims 1-11, wherein a miR-33 antagonist is administered to the subject.

13. The method of claim 12, wherein the miR-33 antagonist is an organic compound having a molecular weight less than 1000 Daltons, a DNA aptamer, an RNA aptamer, an interfering RNA (RNAi) molecule, an antisense oligonucleotide, a ribozyme, or a polypeptide.

14. The method of claim 13, wherein the miR-33 antagonist is an antisense oligonucleotide that targets miR-33, and the antisense oligonucleotide is a morpholino oligomer or has nucleotides in the sequence: TGC AAT GCA ACT ACA ATG CAC (SEQ ID NO: 2).

15. The method of any one of claims 1-14, wherein a TLR4/Myd88 pathway antagonist is administered to the subject.

16. The method of claim 15, wherein the TLR4/Myd88 pathway antagonist is an organic compound having a molecular weight less than 1000 Daltons, a DNA aptamer, an RNA aptamer, an interfering RNA (RNAi) molecule, an antisense oligonucleotide, a ribozyme, a polypeptide, or an antibody.

17. The method of claim 16, wherein the TLR4 Myd88 pathway antagonist is
a) an interfering RNA (RNAi) molecule, an antisense oligonucleotide, or a ribozyme, that i) targets TLR4-encoding mRNA and is capable of reducing TLR4 expression or ii) targets Myd88-encoding mRNA and is capable of reducing Myd88 expression;
b) an anti-TLR4 antibody;
c) an IRAK inhibitor; or
d) a Myd88 blocking peptide.

18. The method of any one of claims 1-17, wherein
a) two or more of the LXR agonist, the miR-33 antagonist, or the TLR4/Myd88 pathway antagonist are administered to the subject; or
b) the LXR agonist, the miR-33 antagonist, or the TLR4/Myd88 pathway antagonist is administered as a monotherapy.

19. The method of any one of claims 1-18, further comprising administering an additional compound to the subject, each of the LXR agonist, the miR-33 antagonist, or the TLR4/Myd88 pathway antagonist and the additional compound being administered in an amount such that, when administered in combination, the administration of the LXR agonist, the miR-33 antagonist, or the TLR4/Myd88 pathway antagonist and the additional compound is effective to treat the subject.

20. The method of claim 19, wherein the additional compound is
a) a steroid;
b) a glucocorticosteroid;
c) other than a steroid;
d) an MMP inhibitor; or
e) a bronchodilator.

21. The method of claim 19 or 20, wherein the additional compound lowers plasma or liver triglycerides in the subject.

22. A method for prophylactically treating a subject for chronic obstructive pulmonary disease (COPD) which comprises administering to the subject i) a Liver X receptor (LXR) agonist, ii) a miR-33 antagonist, or iii) a TLR4/Myd88 pathway antagonist in an amount that is effective to treat the subject.

23. The method of any one of claims 1-22, wherein the subject
a) is a mammalian subject;
b) is a human subject;
c) has a substantially healthy cardiovascular system;
d) has hypercholesterolemia;
e) has abnormal cholesterol efflux in the lungs;
f) has abnormal cholesterol homeostasis in the lungs;
g) is or has been a cigarette smoker; or
h) is afflicted with COPD caused by chronic cigarette smoking.

24. The method of any one of claims 1-23, wherein, if the subject is receiving treatment for a disease other than COPD then the disease other than COPD is other than atherosclerosis.

25. A method for
a) identifying whether a subject afflicted with chronic obstructive pulmonary disease (COPD) is responding to treatment for COPD comprising
i) periodically obtaining biological samples from the subject;
ii) assaying whether the level of a ceramide has increased or decreased in the biological samples over a period of time, and
iii) identifying the subject as responding to treatment if the level of the ceramide has decreased over the period of time;
b) identifying whether a subject afflicted with chronic obstructive pulmonary disease (COPD) is responding to treatment for COPD comprising
i) periodically obtaining biological samples from the subject;
ii) assaying whether the level of sphingosine 1-phosphate (S1P) has increased or decreased in the biological samples over a period of time, and
iii) identifying the subject as responding to treatment if the level of S1P has increased over the period of time;
c) determining whether chronic obstructive pulmonary disease (COPD) is progressing in a subject afflicted with COPD comprising
i) periodically obtaining biological samples from the subject;
ii) assaying whether the level of a ceramide has increased, or decreased in the biological samples over a period of time, and
iii) identifying the COPD as progressing in the subject if the level of the ceramide has decreased over the period of time; or
d) determining whether chronic obstructive pulmonary disease (COPD) is progressing in a subject afflicted with COPD comprising
i) periodically obtaining biological samples from the subject;
ii) assaying whether the level of sphingosine 1-phosphate (S1P) has increased or decreased in the biological samples over a period of time, and
iii) identifying the COPD as progressing in the subject if the level of sphingosine 1-phosphate (S1P) has increased over the period of time.

26. The method of claim 25, comprising treating the subject in accordance with any one of claims 1-76 if in step iii) the COPD is identified as progressing in the subject.

27. The method of claim 25 or 26, wherein the subject is receiving treatment comprising i) a Liver X receptor (LXR) agonist, ii) a miR-33 antagonist, or iii) a TLR4/Myd88 pathway antagonist, and the the subject continues receiving treatment comprising the Liver X receptor (LXR) agonist, the miR-33 antagonist, or the TLR4/Myd88 pathway antagonist if in step iii) COPD is identified as progressing in the subject.

28. The method of any one of claims 25-27, wherein the biological sample is serum or bronchoalveolar lavage fluid.

29. A composition for use
a) in treating a subject afflicted with chronic obstructive pulmonary disease (COPD) which comprises i) a Liver X receptor (LXR) agonist, ii) a miR-33 antagonist, or iii) a TLR4/Myd88 pathway antagonist; or
b) in prophylactically treating a subject for chronic obstructive pulmonary disease (COPD) which comprises i) a Liver X receptor (LXR) agonist, ii) a miR-33 antagonist, or iii) a TLR4/Myd88 pathway antagonist.

30. Use of i) a Liver X receptor (LXR) agonist, ii) a miR-33 antagonist, or iii) a TLR4/Myd88 pathway antagonist for the manufacture of a medicament for a) the treatment of chronic obstructive pulmonary disease (COPD) or b) the prophylactic treatment of COPD.


SEQUENCE LISTING (2 sequences)

SEQ ID NO: 1 - DNA, 23 bp, Artificial Sequence
  Control Vivo-Morpholino oligomer
ttatcgccatgtccaatgaggct

SEQ ID NO: 2 - DNA, 21 bp, Artificial Sequence
  miR-33 targeted Vivo-Morpholino oligomer
tgcaatgcaactacaatgcac